SEQUENCE LISTING (1233 sequences)

SEQ ID NO: 1 - DNA, 70 bp, Homo sapiens
ggaaaacagccggtgatcttctaccaataaagccagtggaaattgccatagaggcatggtgggtggtgca

SEQ ID NO: 2 - DNA, 70 bp, Homo sapiens
tttgcaaatatgtgtataaccacattggtggggagcattccgctgtgatcccagagctggcagccacagt

SEQ ID NO: 3 - DNA, 70 bp, Homo sapiens
cctggcttggagacccctctctgccatctgttgactggctctgtaattctggaaaacaccctttctaaac

SEQ ID NO: 4 - DNA, 70 bp, Homo sapiens
actggtaagaccatcactctcgaggtggagccgagtgacaccattgagaatgtcaaggcaaagatccaag

SEQ ID NO: 5 - DNA, 70 bp, Homo sapiens
tactaaaaatacaaaaattagcccggtgtggtggcaggtgactgtagtcccagctactcggcaggctgag

SEQ ID NO: 6 - DNA, 70 bp, Homo sapiens
aaattgtcctaataatatgtggtgctcatgagtgcgggacctgactgggctcagctaggcggttctcact

SEQ ID NO: 7 - DNA, 70 bp, Homo sapiens
atacaaaaaattagcccagtgtggtggcacatgcctgtagtccctcagacctgtaagctactcaggaggc

SEQ ID NO: 8 - DNA, 70 bp, Homo sapiens
gtatgtagaagacttcaaagccctagaggatggcagagccaccagctggacaaaaactgggcccagaatt

SEQ ID NO: 9 - DNA, 70 bp, Homo sapiens
catcccactccatcccttctgggatgtgaatcatccgtttgtccagcgtattcacgctatatatgctccc

SEQ ID NO: 10 - DNA, 70 bp, Homo sapiens
agatatcgagctcaggactattaagcacgcctgtctacccacagcacagtactgatcattacagggcgca

SEQ ID NO: 11 - DNA, 70 bp, Homo sapiens
actcatacctcccatcttccagctgaagggctctcaagcccgctaagcaagcttctttatttactcggct

SEQ ID NO: 12 - DNA, 70 bp, Homo sapiens
gtaccgccccatgtataaggctttccggagtgacagttcattcaatttcttcgttttcttcttcattttc

SEQ ID NO: 13 - DNA, 70 bp, Homo sapiens
tttagccacagacgtaggctacaagacagcggaacatcactttacggctttgcccacagacatgaaggtg

SEQ ID NO: 14 - DNA, 70 bp, Homo sapiens
cagggcgtagggcctgggccggggtcggcggcgcccccggggctggaggcggcccggcagaagctggcgc

SEQ ID NO: 15 - DNA, 70 bp, Homo sapiens
gttggggtccatccctctctgatgtgctttttccacaacacatatctggtcctctggcaggattgtggat

SEQ ID NO: 16 - DNA, 70 bp, Homo sapiens
ctgtggttggagtccgtgcggctggagtaccgtgcggggctgaagaacatcgcaaatacactcatggcca

SEQ ID NO: 17 - DNA, 70 bp, Homo sapiens
gtccctgggcagccctccatttgagaaacctaatattgagcagggtgtgctgaactttgtgcagtacaag

SEQ ID NO: 18 - DNA, 70 bp, Homo sapiens
acctcgcccatcttcacttagccttcgtatttgtgaaggattcagccaccttccttcttcaccccatgct

SEQ ID NO: 19 - DNA, 70 bp, Homo sapiens
caatgaagatattttagagtacaaaagaagaaatgggctggaataaacttttgaaacactaatgtagtat

SEQ ID NO: 20 - DNA, 70 bp, Homo sapiens
cgcgtcgctagctagtcgttctgaagcggcggccagagaagagtcaagggcacgagcatcgggtagccat

SEQ ID NO: 21 - DNA, 70 bp, Homo sapiens
cgcaagcttggcagcctttggtagagggtagcgagaacaagggaatgttgagagaatatggagagacaga

SEQ ID NO: 22 - DNA, 70 bp, Homo sapiens
atttaccaacctgggggattgatacgaccggggaaaatgttcctaaaccaggaagctgcgttagccgatc

SEQ ID NO: 23 - DNA, 70 bp, Homo sapiens
gcagtgtgggacaaagtccttagacaagaagcagcccagggtatccaataattgaaaaaggaggctgggg

SEQ ID NO: 24 - DNA, 70 bp, Homo sapiens
tgtgggagtatacatcggtgcaggcttcctggatgacagttgggtgatatgtgtcatgtggcctaaaagc

SEQ ID NO: 25 - DNA, 70 bp, Homo sapiens
cccctctcccaggtgtccccttgtagcatatgcattatgtcatctgaattgaggcctttctgtgaacagc

SEQ ID NO: 26 - DNA, 70 bp, Homo sapiens
attttacactttgttactaatttgcagaactctattaattgggtaggatttcacccattcctagctaagt

SEQ ID NO: 27 - DNA, 70 bp, Homo sapiens
tgccacgtatagctggaattaagtgttgtcttggagctgttgtacatttaagaataaacttttgtaaaaa

SEQ ID NO: 28 - DNA, 70 bp, Homo sapiens
tgggtcggtagtagcgatggcgggtctgactgacttgcagcggctacaggcccgagtggaagagctggag

SEQ ID NO: 29 - DNA, 70 bp, Homo sapiens
gtcatccagccctgctgtaaaatatgaagctgctgggacattagtgacactctctagtgcaccaactgca

SEQ ID NO: 30 - DNA, 70 bp, Homo sapiens
cctctgaggagccctcctggatgaatggagggaggcactcggctaacaaattagggcttctcgacgtcct

SEQ ID NO: 31 - DNA, 70 bp, Homo sapiens
caggaagcagcgtctcatcaggacagaaggtaggatgaagacatggggtaatgtgagagagtagaacacc

SEQ ID NO: 32 - DNA, 70 bp, Homo sapiens
tgaatcccactcccaccagagaattagcgcgggcggacgagcaaagtgaaacttagtagcccggaacttc

SEQ ID NO: 33 - DNA, 70 bp, Homo sapiens
ccctggagctgagcacaaagagtcatgtgacsgaagaggaggaggaggaagaggaagaagaatcagattc

SEQ ID NO: 34 - DNA, 70 bp, Homo sapiens
gcgtgagacacatcacatttgtggacaatgccaagatctcctactccaatcctgtgaggcagcctctcta

SEQ ID NO: 35 - DNA, 70 bp, Homo sapiens
agggcctgctccatcccaccttcctttctgctgcctgatgtctcaatggcttctgaatgactgttctaat

SEQ ID NO: 36 - DNA, 70 bp, Homo sapiens
gcggacgctatctacgaccacatcaacgaggggaagctgtggaaacacatcaagcacaagtatgagaaca

SEQ ID NO: 37 - DNA, 70 bp, Homo sapiens
tatgggaccacactgtgctgagaagcttcctgaggcccctcaacctgaaggccctgctacaagcagttca

SEQ ID NO: 38 - DNA, 70 bp, Homo sapiens
gcactcccttggtgtagacaaataccagttcccattggtgttgttgcctataataaacactttttctttt

SEQ ID NO: 39 - DNA, 70 bp, Homo sapiens
gcaccattgaattctgcagttcctagtgctggtgcttccgtgatacagcccagctcatcaccattagaag

SEQ ID NO: 40 - DNA, 70 bp, Homo sapiens
atctgtgcggaagtagcttgcctcacttctgcttaggaaagcggctgttgctccataactctaaccagca

SEQ ID NO: 41 - DNA, 70 bp, Homo sapiens
gcgcgcacgcacgccttgagcagtcagcattgcacctgctatggagaagggtattcctttattaaaatct

SEQ ID NO: 42 - DNA, 70 bp, Homo sapiens
gacctactgtattagacagtaacctctaacctcacctccaagcccaagtatatggccctgctgggttacc

SEQ ID NO: 43 - DNA, 70 bp, Homo sapiens
catatctgtttcctccatcggagcaaaaccactgagatcatccattcaaccctgaatcccacgtgggacc

SEQ ID NO: 44 - DNA, 70 bp, Homo sapiens
ttacatccatctatgagtggaaagggaagatcgaggaagacagtgaggtgctgatgatgattaaaaccca

SEQ ID NO: 45 - DNA, 70 bp, Homo sapiens
gtattcagcctttaggatgatcagaaaagcagaaagagagagtggccggatggggctgaggggagaaaga

SEQ ID NO: 46 - DNA, 70 bp, Homo sapiens
aggccccgcagtccctctcccaggaggaccctagaggcaattaaatgatgtcctgttccattggcaaaaa

SEQ ID NO: 47 - DNA, 70 bp, Homo sapiens
tactaataattattagctacaggcgggcgcagtggctcacaaccgtaatcccagcagtttgggaggctga

SEQ ID NO: 48 - DNA, 70 bp, Homo sapiens
gccgccactccagcctaatcccaaccccagggcgaacgttttcttatttatttccgttttctcgccacta

SEQ ID NO: 49 - DNA, 70 bp, Homo sapiens
ggatctgggcagtcagcactctttttagatctttgtgtggctcctatttttatagaagtggagggatgca

SEQ ID NO: 50 - DNA, 70 bp, Homo sapiens
tcgcccactaagccaatcactttattgactcctagccgcagacctcctcattctaacctgaatcggagga

SEQ ID NO: 51 - DNA, 70 bp, Homo sapiens
gccaatggtggcagcagaagtaggcgtatgggataactattgtgtaaagaaacagcttcttcactcctgc

SEQ ID NO: 52 - DNA, 70 bp, Homo sapiens
cttatgacattatctctaggctgccacttaaagtatggtttgaagacagggagaacggggcggcggagtg

SEQ ID NO: 53 - DNA, 70 bp, Homo sapiens
actagccgtgttttctcagactccacctttgtttgcactctgttgcctgtgaggagctttctggcatgtg

SEQ ID NO: 54 - DNA, 70 bp, Homo sapiens
gaggagctctcgacttagaggtaatatgaacagatgaacagacactgtggctggagccccaaagtgtgga

SEQ ID NO: 55 - DNA, 70 bp, Homo sapiens
tgccccactgagaagggtctagcggagcacaggtcaccagctgggcaacattcagaaagttagtcttcct

SEQ ID NO: 56 - DNA, 70 bp, Homo sapiens
cgggaggacaaccagaccaaccgcctgcaggaggctctgaacctcttcaagagcatctggaacaacagat

SEQ ID NO: 57 - DNA, 70 bp, Homo sapiens
tgaggcatgtactccccatgaggccacacaagagctgtgctttcttagatctggatcccactaccacata

SEQ ID NO: 58 - DNA, 70 bp, Homo sapiens
tgagccaggcctactcgtccagccagcgcgtgtcctcctaccgccgcaccttcggcggggccccgggctt

SEQ ID NO: 59 - DNA, 70 bp, Homo sapiens
cggcttcgaccctatatcccccgcccgcgtccctttctccataaaattcttcttagtagctattaccttc

SEQ ID NO: 60 - DNA, 70 bp, Homo sapiens
gaacagccaagctttgtgctactatgggatttcgttttctgcggttccaagtcttgatccacgtcctgcc

SEQ ID NO: 61 - DNA, 70 bp, Homo sapiens
catgtcatgcagctcagctgggagctgcttaggtggaaaactccaaataaagtgcgcctgtcgcagaaaa

SEQ ID NO: 62 - DNA, 70 bp, Homo sapiens
cccagaagcagttaagtctccaaaacgagtgaaatctccagaaccttctcacccgaaagccgtatcaccc

SEQ ID NO: 63 - DNA, 70 bp, Homo sapiens
ccaggaaagatttgccctcaagaacctcaaatgtagagagaaaagcatctcagcaacaatggggtcgggg

SEQ ID NO: 64 - DNA, 70 bp, Homo sapiens
ctgtggccaggggtccaaacagaaaataaccggagaagacaaggaggtcaaaggatcagggaactaagca

SEQ ID NO: 65 - DNA, 70 bp, Homo sapiens
aaccctggggattgggtgccatctctctaggggtaacacaaagggcaagaggttgctatggtatttggaa

SEQ ID NO: 66 - DNA, 70 bp, Homo sapiens
aagagcgtcaagcagacctgtgacaagtgtaacaccatcatctgggggctcattcagacctggtacacct

SEQ ID NO: 67 - DNA, 70 bp, Homo sapiens
cctctgaccgtttcagcaccctgggttgttaccacgtcctacaactctgacatttcttgttctcaagcgt

SEQ ID NO: 68 - DNA, 70 bp, Homo sapiens
attggtgagctgaagtctgtccttgcaccatgttatcatctgtttctcgtgtccgcctggttgaggagga

SEQ ID NO: 69 - DNA, 70 bp, Homo sapiens
aagctcacctgggcaggtctctgccacctccttgctctgtgagctgtcagtctaggttattctctttttt

SEQ ID NO: 70 - DNA, 70 bp, Homo sapiens
gagaatgatttacaacccctgctagcctggcttaaggtcatggagaaagcccacatcaacctggtgaggt

SEQ ID NO: 71 - DNA, 70 bp, Homo sapiens
cattttctgttgcaggaagccactccaccacagaatgctaatatgccagtggtacccagtacctcttgta

SEQ ID NO: 72 - DNA, 70 bp, Homo sapiens
tcccttgatcattatctctgaagtccctacctgcacttccctgattgccctgtagcaacaccagcatggt

SEQ ID NO: 73 - DNA, 70 bp, Homo sapiens
ttctgacaggaaaggggctccggaaaatcataaaacaagcaggtgaacaagaccaggtgtgtcggcacct

SEQ ID NO: 74 - DNA, 70 bp, Homo sapiens
ttttctcacaagaacccagttagctgatgttttattgtaattgtcttaatttgctaagaacaagtaataa

SEQ ID NO: 75 - DNA, 70 bp, Homo sapiens
gggtttgtgaaaagtgtatgtatttaaatttgctgtaaaacataatcactaataatatgcaataaatatt

SEQ ID NO: 76 - DNA, 70 bp, Homo sapiens
tttgggagagacttgttttggatgccccctaatccccttctcccctgcactgtaaaatgtgggattatgg

SEQ ID NO: 77 - DNA, 70 bp, Homo sapiens
ctccgtgagagcaaggatcctcctgtttaccctgtacctccaatgtctggcacttgtaggtgctcaaata

SEQ ID NO: 78 - DNA, 70 bp, Homo sapiens
ctcggagcagaacccaacctccgagcagtacatgctaagacttcaccagtcaaagcgaactactatactc

SEQ ID NO: 79 - DNA, 70 bp, Homo sapiens
gggactgccagcccctaactgaaatctgaagctttttatcgcttatttttcctcgccctgtctcctccct

SEQ ID NO: 80 - DNA, 70 bp, Homo sapiens
accacggctgtgctactcacggtcatgctggagggatgcagaaactaaatgaatccacagctacttactc

SEQ ID NO: 81 - DNA, 70 bp, Homo sapiens
ctccccagccacaaggagtagaaccagtagctcaaggaattgtttcacagcagttgcctgcagttagttc

SEQ ID NO: 82 - DNA, 70 bp, Homo sapiens
acgaggagacagggaaagtgaaggcccactcacagactgaccgagagaacctgcggatcgcgctccgcta

SEQ ID NO: 83 - DNA, 70 bp, Homo sapiens
cgaaatgaagtttatcataggaaaatcatctcttggtttggtgattcccccttggctctttttggcttac

SEQ ID NO: 84 - DNA, 70 bp, Homo sapiens
gctgaaagatgtactgcagtcagcttcagggcagcttcctgccacagcagcattaaatgaagttggaatt

SEQ ID NO: 85 - DNA, 70 bp, Homo sapiens
gtacttggagttgggacctcacctggctctcccttatctttccggctgccattttttcccctttctaact

SEQ ID NO: 86 - DNA, 70 bp, Homo sapiens
tagatctctaagcccctcctggaaccctcattttccccactctcaatgtcccagtgtccagcgtgactaa

SEQ ID NO: 87 - DNA, 70 bp, Homo sapiens
ccagggactgccccagctgtcctgggcacaagtctctccagcatctttgttcattgattcaacaaagtat

SEQ ID NO: 88 - DNA, 70 bp, Homo sapiens
aataatgcctggtcattgggtgacctgcgattgtcagaaagaggggaaggaagccaggttgatacagctg

SEQ ID NO: 89 - DNA, 70 bp, Homo sapiens
tacggccgcgcctttgtgttcctgtcttctctccaccaccaaaagcaaaagatgatttcccattcactgc

SEQ ID NO: 90 - DNA, 70 bp, Homo sapiens
atcatcatgtcctagcacagatggccccaagcaggggaagtacaatactgcaggctgcaaatccatgtca

SEQ ID NO: 91 - DNA, 70 bp, Homo sapiens
cccagactcaccggacaggataactgtggcctcttcattaaactgcaccgtgttcaccttctgagaaagt

SEQ ID NO: 92 - DNA, 70 bp, Homo sapiens
atttgcatctgaaaggtcccaaggtgaagggcgatgtggatgtttctctgcccaaagtggaaggtgacct

SEQ ID NO: 93 - DNA, 70 bp, Homo sapiens
gatggaaagagtctcacttgcagttgcttcagtcacaacccaggcgtctgccttaatagcatcacctgtg

SEQ ID NO: 94 - DNA, 70 bp, Homo sapiens
aatacagcaattttggcaataactcttatcactcctcagggcttagggtggtcccaggtacccaggggtc

SEQ ID NO: 95 - DNA, 70 bp, Homo sapiens
gaaatggtgcgttggtggtcatacttagtgttctaggctgtgaaatcatggagttcttccacttccaaga

SEQ ID NO: 96 - DNA, 70 bp, Homo sapiens
tgttgggccctgaaaaattagtccgattttgtggtggtaatgggagaaggacatcccaggagcagggtct

SEQ ID NO: 97 - DNA, 70 bp, Homo sapiens
ccatgaccctgaaactagaacaacacgtctcctccctaagtctgcagcttccagatcctcgaattgcaac

SEQ ID NO: 98 - DNA, 70 bp, Homo sapiens
ccctctgccaggcgctagacatgtacagaggtttttctgtggttgtaaatggtcctatttcacccccttc

SEQ ID NO: 99 - DNA, 70 bp, Homo sapiens
gggggagttgagcaggcgccagggctgtcatcaacatggatatgacatttcacaacagtgactagttgaa

SEQ ID NO: 100 - DNA, 70 bp, Homo sapiens
cccatccctaataggctgggctttgcaggaaatggcatgaaatcagctcttctgagtgcacagaagaacc

SEQ ID NO: 101 - DNA, 70 bp, Homo sapiens
gggggttccttcctgttgctaaggtttggaggtgttctgttatttacctgaagtgctgcagctgggaatc

SEQ ID NO: 102 - DNA, 70 bp, Homo sapiens
atcattgaaaggtcctctctgccagcagtggtgccaccctttggtttgctgtggtactttgctgtgtact

SEQ ID NO: 103 - DNA, 70 bp, Homo sapiens
gccggtttttccatgtcatacaaaaaagtcctggctgtttctccgaactggctgcctgcattcccgtctt

SEQ ID NO: 104 - DNA, 70 bp, Homo sapiens
ctgagaggaacctggacatggtcccgggcatctgaatgatctgtaggggagggagttcaaataaagcttt

SEQ ID NO: 105 - DNA, 70 bp, Homo sapiens
tcaaaaccctgagccctgtgcatgctttctcagtcttgtggtgggactggatacaatgactaacttcccc

SEQ ID NO: 106 - DNA, 70 bp, Homo sapiens
ggttgcactggggaggtctgggaagatagctgtttctgaagacttgccgctgtggacacagttaactaaa

SEQ ID NO: 107 - DNA, 70 bp, Homo sapiens
ggagaaagaagagctcgctgtgaaaaacgctccacaatgctgcagagccttgtgaaggtggaagagtact

SEQ ID NO: 108 - DNA, 70 bp, Homo sapiens
ataaagttgttacaaagtgaccttgagtgtcttccttggtgcacccgaaaccccgccttcttcatccggg

SEQ ID NO: 109 - DNA, 70 bp, Homo sapiens
agccagtcctgttggtggaggggatcaccgagagtgtctgtatcattttgtagcccttttctctgacgtt

SEQ ID NO: 110 - DNA, 70 bp, Homo sapiens
gggcatctgagggcagtaaggaacaggtgtccaaaggaggaatgttggtgcctatgagtatgttttccag

SEQ ID NO: 111 - DNA, 70 bp, Homo sapiens
aaatggccaccaccattctccttccccaccccaccacaaaaagagaagctgtgtctttagacaaccctga

SEQ ID NO: 112 - DNA, 70 bp, Homo sapiens
gcccgcagttggagttggactgtcttaacagtagcgtggcacacagaaggcactcagtaaatacttgttg

SEQ ID NO: 113 - DNA, 70 bp, Homo sapiens
ctctgaagcgagctggtttagttgtagaagatgctctgtttgaaactctgccttctgacgtccgggagca

SEQ ID NO: 114 - DNA, 70 bp, Homo sapiens
cccacctgtagatccatagcaacagtggatcagggcaggaagcaagcacataaagtggagtttcccttct

SEQ ID NO: 115 - DNA, 70 bp, Homo sapiens
ctgagcctagagcagggagtcccgaacttctgcattcacagaccacctccacaattgttataaccaaagg

SEQ ID NO: 116 - DNA, 70 bp, Homo sapiens
gcctggggaacgtggttggctcagggtttgacagagaaaagacaaataaatactgtattaataagatgtt

SEQ ID NO: 117 - DNA, 70 bp, Homo sapiens
gcaccgttaggtttcagatctcccgtgtggtgtttgatgtcggcttttgttcctaccttgggagtttgga

SEQ ID NO: 118 - DNA, 70 bp, Homo sapiens
gtaagtaacttgtgctagtcactgggggacctgggtttcagactgggcaatctggctgatcattttccag

SEQ ID NO: 119 - DNA, 70 bp, Homo sapiens
caccttggcctctgaaagtgctagaattacgggcatgagccaccgcatccagccagaaagatacatatct

SEQ ID NO: 120 - DNA, 70 bp, Homo sapiens
atccattctcacatttaaactactgtccagggccgggcgcagtgggtcacgtctgtaattccagcacttt

SEQ ID NO: 121 - DNA, 70 bp, Homo sapiens
gtttggactatagaaatgcggctgttcgctgcaaccaatcaaaaccctctgtggtttaggctagcgggct

SEQ ID NO: 122 - DNA, 70 bp, Homo sapiens
ggccaaagagaacaccagaagacccttaattttacaggcagagttgcctcaggccaatgactggctccaa

SEQ ID NO: 123 - DNA, 70 bp, Homo sapiens
cattctcctaaaggtgactccagtcctgtgctgagtcctgtgcattctcctaaaggtgactctagtcctg

SEQ ID NO: 124 - DNA, 70 bp, Homo sapiens
gcgtcaggagccggctgtgtccttcctgccacactcggggattcattccttagaaactgaaataaattct

SEQ ID NO: 125 - DNA, 70 bp, Homo sapiens
gggcactaatggagatactcatctggggtggagaagactttgaccagcgtgtcattggacacttcatcaa

SEQ ID NO: 126 - DNA, 70 bp, Homo sapiens
ccaggtctctgtagtacttggcaaacctgaaattgtagccaggagatacgttgtgctcaacgtcccgtgt

SEQ ID NO: 127 - DNA, 70 bp, Homo sapiens
ccataaaatgtttctcttctgaacaagccccatcatttggtgaacctccaccctaacaaagtaggatggg

SEQ ID NO: 128 - DNA, 70 bp, Homo sapiens
tcaagtggagcttcatgaataagccctcagatggcaggcccaagtatctggtggtgaacgcagacgaggg

SEQ ID NO: 129 - DNA, 70 bp, Homo sapiens
gctataggttgcagcttggctctatctgctgtctcaataacagcctttgaactgtccacgtatctyaaaa

SEQ ID NO: 130 - DNA, 70 bp, Homo sapiens
tcctccaggtttttcaattaaacggattattttttcagaccgaaaagagatggtctgagtttgtcttaga

SEQ ID NO: 131 - DNA, 70 bp, Homo sapiens
ctgctgccatgtgagtatgtgggcccagtgttgccagatcacctgcttttatacgaagaccctaaactct

SEQ ID NO: 132 - DNA, 70 bp, Homo sapiens
tataaaaattagccagtactaggaaggctgaggcaggataatcgctggaacccgggaggtggaggttgca

SEQ ID NO: 133 - DNA, 70 bp, Homo sapiens
aaaagaaattagctgcacattgtggtgagcgcctgtaatcccagctactcaggaggctgaggcaggagaa

SEQ ID NO: 134 - DNA, 70 bp, Homo sapiens
gcactctcaaatttctacgctcaaacaatccttccacctcaggctcctgagtagctgggactacaggcat

SEQ ID NO: 135 - DNA, 70 bp, Homo sapiens
aaaaaagagcccgcatcgccaagtcaatcttaagccaaaagaacaaagccagaggcatcacactacctga

SEQ ID NO: 136 - DNA, 70 bp, Homo sapiens
aattcccggttctcagaattgttatcactctggtgcatgctgtcacaggggccgttgcgtttggctttgt

SEQ ID NO: 137 - DNA, 70 bp, Homo sapiens
gtggatggatcacaaggtcaggagatcgagaccatcctggctaatacggtgaaaccccgtctctactaaa

SEQ ID NO: 138 - DNA, 70 bp, Homo sapiens
ctatcaaagggtggggtggtgccacctccgtgctgtgcaggagtcaaaaagttgaacggtatggctcaaa

SEQ ID NO: 139 - DNA, 70 bp, Homo sapiens
ttagtgcctgcacctcaccacgatattgaggaagcacaggacatccaagggtactctccagtttggctgt

SEQ ID NO: 140 - DNA, 70 bp, Homo sapiens
tcatcttttagagcagctgccatcacatcggacatattggaggcccttggaagagacggtcacttcacac

SEQ ID NO: 141 - DNA, 70 bp, Homo sapiens
caggccacctactcatgcacctaattggaagcgccaccctagcaatatcaaccattaaccttgcctctac

SEQ ID NO: 142 - DNA, 70 bp, Homo sapiens
  misc_feature (50)..(50): n is a, c, g, or t
aagcccctattttttccaagcacgaagccaccagtcttccccagggagcnatcagnagggacatggatgt

SEQ ID NO: 143 - DNA, 70 bp, Homo sapiens
ctgcgcctgaggggtggctgttaattcttcagtcatggcattcgcagtgcccagtgatggcattactctg

SEQ ID NO: 144 - DNA, 70 bp, Homo sapiens
taggcttaaaaacagatgcaattcccggacgtctaaaccaaaccactttcaccgctacacgaccgggggt

SEQ ID NO: 145 - DNA, 70 bp, Homo sapiens
ctggaaaagggacagactatcagagagttgcactgttgcggtatgggccaaatccaacataatacccgct

SEQ ID NO: 146 - DNA, 70 bp, Homo sapiens
tcgcaccactgcactccagcctgggcaacagagcaagactgtgtcttgacagcaacaaaaaaagaagata

SEQ ID NO: 147 - DNA, 70 bp, Homo sapiens
cgctgatttcctgaaatagagatacccctttgagtgataaatttgcaaaatgctgtcttcattttctgta

SEQ ID NO: 148 - DNA, 70 bp, Homo sapiens
ccgctggggaggtcctccatgcgcagtcatgagtcgcttcaagtttatcgtttatgattacaggtggaaa

SEQ ID NO: 149 - DNA, 70 bp, Homo sapiens
caaatacttttcctgcctccaccaaacccctacagaacatcacctggaattgccactcacactgggttgg

SEQ ID NO: 150 - DNA, 70 bp, Homo sapiens; identical to SEQ ID NO: 13

SEQ ID NO: 151 - DNA, 70 bp, Homo sapiens
ggatattctgttggtgataccaaacaccaaggggcctccagctgggtttcagtagtacgatgagtcactg

SEQ ID NO: 152 - DNA, 70 bp, Homo sapiens
catccaaacccagtcgtctgccctggatcgttttaatgccatgaactcagccttggcgtcagattccatt

SEQ ID NO: 153 - DNA, 70 bp, Homo sapiens
tttctttagacccatacttactgttcctcaaatgcctgcagtttgcccgggagtcgtctctgcaactggc

SEQ ID NO: 154 - DNA, 70 bp, Homo sapiens; identical to SEQ ID NO: 50

SEQ ID NO: 155 - DNA, 70 bp, Homo sapiens
gggtaccacccaagtattgactcacccatcaacaaccgccatgtatttcgtacattactgccagccacca

SEQ ID NO: 156 - DNA, 70 bp, Homo sapiens; identical to SEQ ID NO: 50

SEQ ID NO: 157 - DNA, 70 bp, Homo sapiens
taagcagtgatctttgctgctgctttccccctttgtctgcccttaggtcactaaggattgtagggccttc

SEQ ID NO: 158 - DNA, 70 bp, Homo sapiens
aagcacaagactgacctcaaccatgaaaacctcaagggtggagacgacctggaccccaactacgtgctca

SEQ ID NO: 159 - DNA, 70 bp, Homo sapiens
tgtgaggttttacagtattctgcaagggaagctcaagattcaaaaaaggtggtagaggacattgaatacc

SEQ ID NO: 160 - DNA, 70 bp, Homo sapiens
agaaatggatgtgggaacagatgaagaagaaacagcaaaggaatctacagctgaaaaagatgaattgttg

SEQ ID NO: 161 - DNA, 70 bp, Homo sapiens
cctacctgccaacctctcctctgctggcagattgtatcatccccattactgatatcagggctttcacaac

SEQ ID NO: 162 - DNA, 70 bp, Homo sapiens
aagtgtgacaacttgatctactagcgaggctgcatggggaaacaggcactttcataggtagctggtggga

SEQ ID NO: 163 - DNA, 70 bp, Homo sapiens
ttgtaatccaggacatctgatctcctacatcaaaaactccaatggggccaggtgtggtggcacttgcctg

SEQ ID NO: 164 - DNA, 70 bp, Homo sapiens
gccatgaaggcactgagtctgtctggtttcctgagggttaaaagattagggctgggatcaccacagcatt

SEQ ID NO: 165 - DNA, 70 bp, Homo sapiens
gacctccccagcatccctgaggtgtggctgcttagttttcgatacttaccttgttaccagatgtcagact

SEQ ID NO: 166 - DNA, 70 bp, Homo sapiens
gaattgcccagtgctgccagagtgagtgagtgtaattctcctttcaggtaaagataggctatctcaacac

SEQ ID NO: 167 - DNA, 70 bp, Homo sapiens
aaatagggctggatcttatcactgccctgtctccccttgtttctctgtgccagatcttcagtgccccttt

SEQ ID NO: 168 - DNA, 70 bp, Homo sapiens
atgtcataacttctgttactcctttggcccatagctaaggtcatccttccccacaggggtggctttggga

SEQ ID NO: 169 - DNA, 70 bp, Homo sapiens
cttgccagaagatgatcttagagttgttttctaaggtgccatccttggtaggaagctttattagaagcca

SEQ ID NO: 170 - DNA, 70 bp, Homo sapiens
ctggtcaccgtttcaccatcatgctttgatgttcccctgtctttccctcttctgctctcaagagcaaagg

SEQ ID NO: 171 - DNA, 70 bp, Homo sapiens
agagtgttgtccagatgtttctgtactggcatagaaaaaccaaataaaaggcctttatttttaaacaaaa

SEQ ID NO: 172 - DNA, 70 bp, Homo sapiens
aatggaaacatctgccccacgtgccggaagccaagtggtggcgacaactgcgcgccactccgcggcctac

SEQ ID NO: 173 - DNA, 70 bp, Homo sapiens
tagtgccactaacggttgagttttgactgcttggaactggaatcctttcagcaagacttctctttgcctc

SEQ ID NO: 174 - DNA, 70 bp, Homo sapiens
taatcctgccagtctttctcttcaagccagggtgcatcctcagaaacctactcaacacagcactctaggc

SEQ ID NO: 175 - DNA, 70 bp, Homo sapiens
tttcacatatgttgtgaattttccttggttctttttaaaggaatgataataaagttacttgctttaggaa

SEQ ID NO: 176 - DNA, 70 bp, Homo sapiens
gggtgtccgctgctgctttccttcggaatccagtgcttccacagagattagcctgtagcttatatttgac

SEQ ID NO: 177 - DNA, 70 bp, Homo sapiens
ctgttgcaactcggctgttctggactctgatgtgtgtggagggatggggaatagaacattgactgtgttg

SEQ ID NO: 178 - DNA, 70 bp, Homo sapiens
gattgctgtgtaccctgcctttgaagcacctcctcagtacgttttgccaacctatgaaatggccgtgaaa

SEQ ID NO: 179 - DNA, 70 bp, Homo sapiens
cacacgtagtggcttaaagcaacgaacattcactctctcacagtctgtgtcagtcggggatttgggagtg

SEQ ID NO: 180 - DNA, 70 bp, Homo sapiens
tcctctaactaggactccctcattcctagaaatttaaccttaatgaaatccctaataaaactcagtgctg

SEQ ID NO: 181 - DNA, 70 bp, Homo sapiens
gaaatgggtccctgggtgacatgtcagatctttgtacgtaattaaaaatattgtggcaggattaatagca

SEQ ID NO: 182 - DNA, 70 bp, Homo sapiens
attattgcaaatactatgggtaccgcaatccttcctgtgaggatgggcgccttcgggtgttgaagcctga

SEQ ID NO: 183 - DNA, 70 bp, Homo sapiens
aagctacactcaaagacactcccaccaggctctttctcccttttcctcttgctcactgccctggaatcaa

SEQ ID NO: 184 - DNA, 70 bp, Homo sapiens
acttgaaaaattacacctggcagctgcgtttaagccttcccccatcgtgtactgcagagttgagctggca

SEQ ID NO: 185 - DNA, 70 bp, Homo sapiens
ccaatcttttacaaagcatgggagtgcagctgcctgacaacaccgatcacagaccaacaagtaagccaac

SEQ ID NO: 186 - DNA, 70 bp, Homo sapiens
cccagctcatccagggagggcggcttatcaaacacgagatgactaaaacggcatctgcataacaatggaa

SEQ ID NO: 187 - DNA, 70 bp, Homo sapiens
atttggatctcacgctgcctctgtggttccctccctcatttttcctggacgtgatagctctgcctattac

SEQ ID NO: 188 - DNA, 70 bp, Homo sapiens
tggaggcctgtggtttccgcacccgctgccacccccgcccctagcgtggacatttatcctctagcgctca

SEQ ID NO: 189 - DNA, 70 bp, Homo sapiens
ggacaagaaagaaatggccatcaatgactgcagcaaagcaattcaattaaaccccagctatatcagggca

SEQ ID NO: 190 - DNA, 70 bp, Homo sapiens
gtccccaacctagcttggtgagggctgtaactgtttccaagtacttgtacattggaagtctgaatgtgta

SEQ ID NO: 191 - DNA, 70 bp, Homo sapiens
ggctggctaactcgtaggaagagagcactgtatggtatccttttgctttattcaccagcattttggggga

SEQ ID NO: 192 - DNA, 70 bp, Homo sapiens
gcggccggcatcatgaccctgtttcacttcgggaactgcttcgctcttgcctacttcccctacttcatca

SEQ ID NO: 193 - DNA, 70 bp, Homo sapiens
atcatgcatgaagcgccaaagatgcaccatgtagaattttcactttgtactggcaggctcgttttacctc

SEQ ID NO: 194 - DNA, 70 bp, Homo sapiens
tctcctctagaccaaggcaggcagccccgacatctgcttcctctatcgcccaatgcaaaatcgatgaaat

SEQ ID NO: 195 - DNA, 70 bp, Homo sapiens
ggtccggtgaccccctggccccagatggcactgagtttttcattcattgaagatttgatttccttgaaaa

SEQ ID NO: 196 - DNA, 70 bp, Homo sapiens
caaggtactctggtgagtcaccacttcagggctttactccgtaacagattttgttggcatagctctgggg

SEQ ID NO: 197 - DNA, 70 bp, Homo sapiens
gaaattagggcctcctctgatctctcgctatctgcgggtcctgtccttttctcaagaccttcaccattac

SEQ ID NO: 198 - DNA, 70 bp, Homo sapiens
ccttccttgccaggacctagagtttgttcagttccaccccacaggcatatatggtgctggttgtctcatt

SEQ ID NO: 199 - DNA, 70 bp, Homo sapiens
gaagatggagacaccctctgggggtcctctctgagtcaaatccagtggtgggtaattgtacaataaattt

SEQ ID NO: 200 - DNA, 70 bp, Homo sapiens
gtgtagggaaaaggatccactgggtgaatcctccctctcagaaccaataaaatagaattgaccttttaaa

SEQ ID NO: 201 - DNA, 70 bp, Homo sapiens
tcaggctttctgtgcatgtactaaaaaaggagaaattataataaattagccgtcttgcggcccctaggcc

SEQ ID NO: 202 - DNA, 70 bp, Homo sapiens
ggtgctaggagaggatggtctccacccatctttctatttccagtacacgtcacattattttaccggtgag

SEQ ID NO: 203 - DNA, 70 bp, Homo sapiens
ggccaaaaacatacagaggtgcatggctggcagtcttgaaattgtcactcgcttactggatccaagcgtc

SEQ ID NO: 204 - DNA, 70 bp, Homo sapiens
tttcccctgctcggaagggttggcctgcctggctggggaggtcagtaaactttgaatagtaagccaaaaa

SEQ ID NO: 205 - DNA, 70 bp, Homo sapiens
aacatggtattaaactctataaacctctcattctccctgtgactcaggccccaatcttcatctccttctt

SEQ ID NO: 206 - DNA, 70 bp, Homo sapiens
ggcactgtgcatattttcaaccagatcaccaggagctgagatcttcttcagtccctagccaggaataccc

SEQ ID NO: 207 - DNA, 70 bp, Homo sapiens
aattcggcacgaggcccgacgctgtggttgctgtaaggggtcctccctgcgccacacggccgtcgccatg

SEQ ID NO: 208 - DNA, 70 bp, Homo sapiens
agatggacgtgcacattactccggggacccatgcctcagagcatgcagtgaacaagcaacttgcagataa

SEQ ID NO: 209 - DNA, 70 bp, Homo sapiens
actgaggggcaagattagcgagcaggacaaaaacaagatcctcgacaagtgtcaggaggtgatcaactgg

SEQ ID NO: 210 - DNA, 70 bp, Homo sapiens
gccaccagagactgagtggaaatcgccccttttgaaggtgccattcttatgagccaaaagtttgtcattt

SEQ ID NO: 211 - DNA, 70 bp, Homo sapiens
cagtatgagaaaaatattcaagtaacactttaaaaccagttacccaaaatctgattagaagtataaggtg

SEQ ID NO: 212 - DNA, 70 bp, Homo sapiens
cggccatgcctttcttggacatccagaaaaggttcggccttaacatagatcgatggttgacaatccagag

SEQ ID NO: 213 - DNA, 70 bp, Homo sapiens
gaggttgctcagctcaagaaaagtgcagataccctgtgggacatccagaaggacctaaaagacctgtgac

SEQ ID NO: 214 - DNA, 70 bp, Homo sapiens
caaaggaaatcagcagtgatagatgaagggttcgcagcgagagtcccggacttgtctagaaatgagcagg

SEQ ID NO: 215 - DNA, 70 bp, Homo sapiens
tatcagaggtgtggaagaagaggaagaagatggggaaatgagagaatagcatcttttgtgggggattttt

SEQ ID NO: 216 - DNA, 70 bp, Homo sapiens
gaacaagtggttcttccagaaactgcggttttagatgctttgttttgatcattaaaaattataaagaaaa

SEQ ID NO: 217 - DNA, 70 bp, Homo sapiens
agggatccactgtgcggtgccaaaaaagaggcggaggctcgcggcacagctctcccggcgcagctctcgg

SEQ ID NO: 218 - DNA, 70 bp, Homo sapiens
aatgttctccgaaacaggatcaacgataaccagaaagtctccaagacccgcgggaaggctaaagtcaccg

SEQ ID NO: 219 - DNA, 70 bp, Homo sapiens
agttccttcttgaaccctggtgcctcctaccctatggccctgaatggtgcactggtttaattgtgttggt

SEQ ID NO: 220 - DNA, 70 bp, Homo sapiens
aggttttcattcgcacggaacaccttttggcatgcttaacttcctggtaacaccttcacctgcattggtt

SEQ ID NO: 221 - DNA, 70 bp, Homo sapiens
ccagcccttaaaatgaaattaacttcctactcaggcaccctgcttaggtgcacagctgttcaatatacac

SEQ ID NO: 222 - DNA, 70 bp, Homo sapiens
aggacagtcatcagaggctctcaggctgagctcaagtgccccgtgtgtcttttggaatttgaggaggagg

SEQ ID NO: 223 - DNA, 70 bp, Homo sapiens
tgacgacttcgccgcgcgttggtcagccatggccaccgctctcgcgctacgtagcttgtaccgagcgcga

SEQ ID NO: 224 - DNA, 70 bp, Homo sapiens
gcctagagccttcagtcactggggaaagcagggaagcagtgtgaactctttattcactcccagcctgtcc

SEQ ID NO: 225 - DNA, 70 bp, Homo sapiens
attatatccccattaaggcaactgctacaccctgctttgtattctgggctaagattcattaaaaactagc

SEQ ID NO: 226 - DNA, 70 bp, Homo sapiens
ccttctgtgacatgtgtttataaaaaatggttaagtatataataaattgaacatctttgagattggagaa

SEQ ID NO: 227 - DNA, 70 bp, Homo sapiens
gccgccatgggagtggagggctgcaccaagtgcatcaagtacctgctcttcgtcttcaatttcgtcttct

SEQ ID NO: 228 - DNA, 70 bp, Homo sapiens
cccttggggaggggccacctgtagtatttgccttgatttggtggggtacagtggatgtgaatactgtaaa

SEQ ID NO: 229 - DNA, 70 bp, Homo sapiens
ctatggttggatctcagctggaagttctgtttggagcccatttctgtgagaccctgtatttcaaatttgc

SEQ ID NO: 230 - DNA, 70 bp, Homo sapiens
gggaccctgttacagacataccctatgccactgctcgagccttcaagatcattcgtgaggcttacaagaa

SEQ ID NO: 231 - DNA, 70 bp, Homo sapiens
ttaaggaacgctagcagggcatggcacgtgagctccggaatagatgtcttcatcacttcttccactgtgt

SEQ ID NO: 232 - DNA, 70 bp, Homo sapiens
aatgtctgtcagtaacgaggcttttgatgtgttgagctggaggtgagtggaccgggggctgtgttttaag

SEQ ID NO: 233 - DNA, 70 bp, Homo sapiens
gcgccgctgagttgtctggccccgccgacccacggcccacgacccaccgacccacgaatcggcccggccg

SEQ ID NO: 234 - DNA, 70 bp, Homo sapiens
ccgatactcccagatctgtgcaaaagcagtgagagatgcactgaagacagaattcaaagcaaatgctgag

SEQ ID NO: 235 - DNA, 70 bp, Homo sapiens
gcaccctcctgaaaactgcagcttccttctcaccttgaagaataatcctagaaaactcacaaaatgtgtg

SEQ ID NO: 236 - DNA, 70 bp, Homo sapiens
ggagtttctgactaatcaaagctggtatttccccgcatgtcttattcttgcccttcccccaaccagtttg

SEQ ID NO: 237 - DNA, 70 bp, Homo sapiens
atttacaagacaggttttaactcagccgaggtgggaaatggtgtccctgtccctcccaaagcacagagca

SEQ ID NO: 238 - DNA, 70 bp, Homo sapiens
ccttgcttctgactttcgcctctgggacaagtaagtcaatgtgggcagttcagtcgtctgggttttttcc

SEQ ID NO: 239 - DNA, 70 bp, Homo sapiens
tgaagcagatgatgaaaactctcaacaacgacctgggccccaactggcgggacaagttggaatacttcga

SEQ ID NO: 240 - DNA, 70 bp, Homo sapiens
tcatttcctcaatgggacggagcgagtgtggaacctgatcagatacatctataaccaagaggagtacgcg

SEQ ID NO: 241 - DNA, 70 bp, Homo sapiens
ctgcagagaagaaacctactacagaggagaagaagcctgctgcataaactcttaaatttgattattccat

SEQ ID NO: 242 - DNA, 70 bp, Homo sapiens
gaccatttggaagaaaagatgcctttagaagatgaggtcgtgcccccacaagtgctcagtgagccgaatg

SEQ ID NO: 243 - DNA, 70 bp, Homo sapiens
gaatgagacatccagcagatttccagccttctactgctctcctccacctcaactccgtgcttaaccaaag

SEQ ID NO: 244 - DNA, 70 bp, Homo sapiens
tagttcttcaccttttaaattatgtcactaaactttgtatgagttcaaataaatatttgactaaatgaaa

SEQ ID NO: 245 - DNA, 70 bp, Homo sapiens
ccgaggaagatactgagggagcacaggagcagtcaccgctgccactgctactgccgctactgctgccggc

SEQ ID NO: 246 - DNA, 70 bp, Homo sapiens
ggggcagcactgggcctggccccccgggtatttattgctgtacatagtgtatgtttgtgatatataaggt

SEQ ID NO: 247 - DNA, 70 bp, Homo sapiens
caaacattagatcctaacaatatgaccatactcaataggacttttcaagatgagccactaattatggatt

SEQ ID NO: 248 - DNA, 70 bp, Homo sapiens
gaggggaagccacttaataaggagtcagacctaaaagggggtgggggacattttcttacctcacccaaga

SEQ ID NO: 249 - DNA, 70 bp, Homo sapiens
aatccactcacgttcataaagagaatgttgatggcgccgtgtagaagccgctctgtatccatccacgcgt

SEQ ID NO: 250 - DNA, 70 bp, Homo sapiens
gccatcctaagattaggacttcttcttgactgcccgagactcgccatttctgcccgtgaatttgtgtctg

SEQ ID NO: 251 - DNA, 70 bp, Homo sapiens
taaagcaaggggaccttggcactctcagctttccctgccacatccagcttgttgtcccaatgaaatactg

SEQ ID NO: 252 - DNA, 70 bp, Homo sapiens
gagggctcactgagaaccatcccagtaacccgaccgccgctggtcttcgctggacaccatgaatcacact

SEQ ID NO: 253 - DNA, 70 bp, Homo sapiens
ctatgaatctttgtgagcaattatgctcccaaatctaagcaagtaaaatacacattttgtctttcttaaa

SEQ ID NO: 254 - DNA, 70 bp, Homo sapiens
acaacaggcatttaagcaatgaagatatgtttagagaagtggatgaaatagatgagataaggagagtcag

SEQ ID NO: 255 - DNA, 70 bp, Homo sapiens
taaccaggccagtgacagaaatggattcgaaataccagtgtgtgaagctgaatgatggtcacttcatgcc

SEQ ID NO: 256 - DNA, 70 bp, Homo sapiens
aatctggcagccagttccgtcctgacagagttcacagcatatattggtggattcttgtccatagtgcatc

SEQ ID NO: 257 - DNA, 70 bp, Homo sapiens
ctgccccctgaaacttatttttttctgattgtaacgttgctgtgggaacgagaggggaagagtgtactgg

SEQ ID NO: 258 - DNA, 70 bp, Homo sapiens
aacaaatggtacagtcataagagccatctgtcacggacccacgcccagaggaacgtgcagaaaaaagcag

SEQ ID NO: 259 - DNA, 70 bp, Homo sapiens
aggatagttggcttcctgcctctctcctctaaaatagcaagtctgggaaatcctggggtgagtggagtca

SEQ ID NO: 260 - DNA, 70 bp, Homo sapiens
tgcgattggttcttctgccatggcttcaacaagtggcctagtaatcacctctccttccaacctcagtgac

SEQ ID NO: 261 - DNA, 70 bp, Homo sapiens
gctcccagcacactcggagcttgtgctttgtctccacgcaaagcgataaataaaagcattggtggcctta

SEQ ID NO: 262 - DNA, 70 bp, Homo sapiens
ccacatatatgcgaatctataagaaaggtgatattgtagacatcaagggaatgggtactgttcaaaaagg

SEQ ID NO: 263 - DNA, 70 bp, Homo sapiens
ttcagtcagcctcagaggttgacttctacattgataaggacatgatccacatcgcggacaccaaggtcgc

SEQ ID NO: 264 - DNA, 70 bp, Homo sapiens
ccttccattttcccccactactgcagcacctccaggcctgttgctatagagcctacctgtatgtcaataa

SEQ ID NO: 265 - DNA, 70 bp, Homo sapiens
gcacctctagtgctactgctagatatcacttactcagttagaattttcctaaaaataagctttatttatt

SEQ ID NO: 266 - DNA, 70 bp, Homo sapiens
acgctcactgcctggcttggaaaagttaagaagcccctcaggaagagaatcgaggccaagttcctctgcg

SEQ ID NO: 267 - DNA, 70 bp, Homo sapiens
ggcttttgaatcgtaatagcaatgtgagggtgaggtacacctacagacattaaataatttgctgtgaaaa

SEQ ID NO: 268 - DNA, 70 bp, Homo sapiens
tcgcctacacaattctccgatccgtccctaacaaactaggaggcgtccttgccctattactatccatcct

SEQ ID NO: 269 - DNA, 70 bp, Homo sapiens
ttcatctctggatgacaagccccagttcccaggggcctcggcggagtttatagataagttggaattcatc

SEQ ID NO: 270 - DNA, 70 bp, Homo sapiens
gcactgctctcagactatgttctccacaacagcaacaccatgagacttggttccatctttgggctaggct

SEQ ID NO: 271 - DNA, 70 bp, Homo sapiens
aagtggtggaatcggctatccataccctcgtgcccctgtttttcctggccgtggtagttactcaaacaga

SEQ ID NO: 272 - DNA, 70 bp, Homo sapiens
gtttaacactaaaccaaggtcatgagcattcgtgctaagataacagactccagctcctggtccacccggc

SEQ ID NO: 273 - DNA, 70 bp, Homo sapiens
tagtgtcagtcaccaaagaaggcctggaacttccagaggatgaagaagagaaaaagaagcaggaagagaa

SEQ ID NO: 274 - DNA, 70 bp, Homo sapiens
cccactgtctggggcagggggagaaggtattttcgagataaagcacaggcaccacaaataaaagtcgtga

SEQ ID NO: 275 - DNA, 70 bp, Homo sapiens
gaggtaatctgggtgcacagaatttatctgagtctgctgctgtgaaggagatactgaaggagcaggaaaa

SEQ ID NO: 276 - DNA, 70 bp, Homo sapiens
acagtcatgcgcagggacgatccttgttctctgctgtaaactgtaaaaagtttatggagacttaaagtct

SEQ ID NO: 277 - DNA, 70 bp, Homo sapiens
tactggaacagccagaaggacatcctggaagacaagcgggccgcggtggacacctactgcagacacaact

SEQ ID NO: 278 - DNA, 70 bp, Homo sapiens
cacctgaggtcgggagttcgcgaccagcctgaccaacatggaaaaaccccgtctctactaaaaatacaaa

SEQ ID NO: 279 - DNA, 70 bp, Homo sapiens
agtcgggctacccactgattttccttcccttacttcccctgagcccttgggcccacttcccagcctaccg

SEQ ID NO: 280 - DNA, 70 bp, Homo sapiens
cagagaaacggcaggaagacccttactactgtccaagggatcgctgatgattacgataaaaagaaactag

SEQ ID NO: 281 - DNA, 70 bp, Homo sapiens
ccccatcttaactgatttaacccctgaaacaacccgacgctggaagttgggttctcatccccactctaca

SEQ ID NO: 282 - DNA, 70 bp, Homo sapiens
tgggctaccatctgcatggggctggggtcctcctgtgctatttgtacaaataaacctgaggcaggaaaaa

SEQ ID NO: 283 - DNA, 70 bp, Homo sapiens
gggcccaattcttctccacgacaatgcccgaccgcatgttgcacaacccacacttcaaaagttgaatgaa

SEQ ID NO: 284 - DNA, 70 bp, Homo sapiens
cacgtctgacagccatgtccacctgtgcccacagcttccgcccacagacctccagggacaggagcaaatt

SEQ ID NO: 285 - DNA, 70 bp, Homo sapiens
ttaaaaaagttgggttttctccattcaggattctgttccttaggattttttccttctgaagtgtttcacg

SEQ ID NO: 286 - DNA, 70 bp, Homo sapiens
gaggggaggggcctagggagccgcaccttgtcatgtaccatcaataaagtaccctgtgctcaaccaaaaa

SEQ ID NO: 287 - DNA, 70 bp, Homo sapiens
ggatactgcgagtatggcggcgtcaaaggtgaagcaggacatgcctccgccggggggctatgggcccatc

SEQ ID NO: 288 - DNA, 70 bp, Homo sapiens
ttaggttaggagttcatagttggaaaacttgtgcccttgtatagtgtccccatgggctcccactgcagcc

SEQ ID NO: 289 - DNA, 70 bp, Homo sapiens
ggcctcaagaggtttggagcaggtatgttaagaagttaggggattttgctaagccggagaatattgactt

SEQ ID NO: 290 - DNA, 70 bp, Homo sapiens
gatcttccctgtctcacacttcttttctcccatcccggttgcaatctcactcagacatcacagtaccacc

SEQ ID NO: 291 - DNA, 70 bp, Homo sapiens
acagattgttcctcccattccccttgccgctttttgcctatcgatgggtagcaagagtctttgaaataag

SEQ ID NO: 292 - DNA, 70 bp, Homo sapiens
gggcccccagcctcatctccggctccagcccctaagttttctccagtgactcctaagtttactcctgtgg

SEQ ID NO: 293 - DNA, 70 bp, Homo sapiens
ccttcagctaatttctgctcccctgagattcgtccttcagccccatcatgtgctttgggatgagtgtaaa

SEQ ID NO: 294 - DNA, 70 bp, Homo sapiens
agtggcccatctttgttggcctacgaactttggtttgatgccagtcaggtgccacatgagaacctttgct

SEQ ID NO: 295 - DNA, 70 bp, Homo sapiens
ccccctgccctcccctctctgcaccgtactgtggaaaagaaacacgcacttagtctctaaagagtttatt

SEQ ID NO: 296 - DNA, 70 bp, Homo sapiens
acaaatgcgacgaacctctgaacatcctggtgaggaataacaagggccgcagcagcacctacgaggtgcg

SEQ ID NO: 297 - DNA, 70 bp, Homo sapiens
tagcccaggctgtggaggggcccagtgagaatgtcaggaagctgtctcgtgcagacttgaccgagtacct

SEQ ID NO: 298 - DNA, 70 bp, Homo sapiens
ctcaattttgtgaggctgtgttggaaataacccgcctctagtgctgttggtatgcaaggcagcggtgctt

SEQ ID NO: 299 - DNA, 70 bp, Homo sapiens
tgctcaaattaccctccaaaagcaagtagccaaagccgttgccaaaccccacccataaatcaatgggccc

SEQ ID NO: 300 - DNA, 70 bp, Homo sapiens
gactccgctgggagagtgcaggagcacgtgctgttttttatttggacttaacttcagagaaaccgctgac

SEQ ID NO: 301 - DNA, 70 bp, Homo sapiens
cgcagcttagagagactcaccagcgagcgtcattgttgtctttctgggaactcattcccatgagatcaga

SEQ ID NO: 302 - DNA, 70 bp, Homo sapiens
cagtggaactgtcccacaagaattcacaggtctcaaagcaggaacagtgggtttgtgtctcacctgagta

SEQ ID NO: 303 - DNA, 70 bp, Homo sapiens
agctagtgccgactcccgcctagctcttttgactctgttcgcgggaagaatggggaaacagtaaggttgc

SEQ ID NO: 304 - DNA, 70 bp, Homo sapiens
acactgtttggaagaaagctaaaccctgaagatcagtagcccctaatcacatgtgctgcaaatagccttc

SEQ ID NO: 305 - DNA, 70 bp, Homo sapiens
ttgtggtcggggagctggggtacaggtttggggagggggaagagaaatttttatttttgaacccctgtgt

SEQ ID NO: 306 - DNA, 70 bp, Homo sapiens
gatctggttacctgtgcagttgtgaatacccagaggttgggcagatcagtgtctctagtcctacccagtt

SEQ ID NO: 307 - DNA, 70 bp, Homo sapiens
tgctccaactgaccctgtccatcagcgttctataaagcggccctcctggagccagccacccagagcccgc

SEQ ID NO: 308 - DNA, 70 bp, Homo sapiens
ccccgcttccccagtctttaaacattggacgctatttactcagctacccagtagagcttgaagctgacct

SEQ ID NO: 309 - DNA, 70 bp, Homo sapiens
ctttcagtctttatgtcacctcaggagacttatttgagaggaagccttctgtacttgaagttgatttgaa

SEQ ID NO: 310 - DNA, 70 bp, Homo sapiens
aggcccctgctggattggcaggccctgtccgaggagttgggggaccatcccagcaggtaatgactccaca

SEQ ID NO: 311 - DNA, 70 bp, Homo sapiens
aagacagcgccgcccgcgcaccgccagcgacccccgccgcagagtcccaccgccacaggcctcgggccag

SEQ ID NO: 312 - DNA, 70 bp, Homo sapiens
tctgttctgtttgtacatggctgacggaaatctctttggtacaaccgaataaagcctggtggcagtgctg

SEQ ID NO: 313 - DNA, 70 bp, Homo sapiens
ccaagtaccataggacagtcacataggagcgtgtagtcgtgactgaataaagaaagcaaaagcctgaaaa

SEQ ID NO: 314 - DNA, 70 bp, Homo sapiens
agtggctaaattgcagtagcagcatatctttttttctttgcacaaataaacagtgaattctcgtttaaaa

SEQ ID NO: 315 - DNA, 70 bp, Homo sapiens
aagcatcttgcttggttgctacattctggtgtgatgggtgcagtggtggctcctctgacaatattagggg

SEQ ID NO: 316 - DNA, 70 bp, Homo sapiens
ttttaattggagaagggtatagaggtagtccaggtgggaacgccagaagtgctgattgcccagccattgg

SEQ ID NO: 317 - DNA, 70 bp, Homo sapiens
aagcctgtgagatcttgtgttgcagcgtggtttggccctagcgttcttgcatgctaacctaaggtagaag

SEQ ID NO: 318 - DNA, 70 bp, Homo sapiens
aaaagcgtacaaaagatacttaaaagggctcctggggtacacaagcccagcaggtcctgagtgaagccgt

SEQ ID NO: 319 - DNA, 70 bp, Homo sapiens
aagacctggaccagtctcctctggtctcgtcctcggacagcccaccccggccgcagcccgcgttcaagta

SEQ ID NO: 320 - DNA, 70 bp, Homo sapiens
ggggcatgcaccctcctttctgtaccgtgtgtgctggctccatagttctctcttctgtacatataagcat

SEQ ID NO: 321 - DNA, 70 bp, Homo sapiens
gaaggctcagcctcaagattcacagcatctcagacacagcctaggccgcaccaggatgtcggacaccgag

SEQ ID NO: 322 - DNA, 70 bp, Homo sapiens; identical to SEQ ID NO: 99

SEQ ID NO: 323 - DNA, 70 bp, Homo sapiens
tcagccagcaccaagccttgttgggcactatcagggctgagggaaagatctcagaacaatcagatgcaaa

SEQ ID NO: 324 - DNA, 70 bp, Homo sapiens
ggccacgggaacaggaccatggttaagcaaccatatagaaagctttgttgaaagaaagtatggcatcttg

SEQ ID NO: 325 - DNA, 70 bp, Homo sapiens
acaacttggagaaatttggaaaactcagtgcgttccccgaacctcctgaggatgggacgctgctatcgga

SEQ ID NO: 326 - DNA, 70 bp, Homo sapiens
cccatggggggtggatgatttgcactttggttccctgtgttttgatttctcattaaagttcctttccttc

SEQ ID NO: 327 - DNA, 70 bp, Homo sapiens
tgggtcctgggaatgctgctgcttcaaccccagagcctaagaatggcagccgtttcttaacatgttgaga

SEQ ID NO: 328 - DNA, 70 bp, Homo sapiens
tggcaaaaacggccaggtacaacacctttttcatacaaggcccaggaggcttagtccagtctgtgctcct

SEQ ID NO: 329 - DNA, 70 bp, Homo sapiens
gcccactgtagtatccacagtgcccgagttctcgctggttttggcaattaaacctccttcctactggttt

SEQ ID NO: 330 - DNA, 70 bp, Homo sapiens
gagcaaaagaccgtgagtcccctagaagttactcatccactttgactgacatggggagaagtgcaccaag

SEQ ID NO: 331 - DNA, 70 bp, Homo sapiens
ggggtgagtgtagttctggcctagcagcaccctcttgtggcttgttctagcgtgtattaaaacttgacac

SEQ ID NO: 332 - DNA, 70 bp, Homo sapiens
gtgtgagagtgtgaatgcacaggtgggtatttaatctgtattattccccgttcttggaattttcttcccc

SEQ ID NO: 333 - DNA, 70 bp, Homo sapiens
gccttccctcagtgatgggttcagttccggaaggtgtcttagaggacattaaagcgcgtacttgctttgt

SEQ ID NO: 334 - DNA, 70 bp, Homo sapiens
ccatatgtcactgggggaaaggctgcctgtacctctcaagctttgcattttactggaaactgaggcgtca

SEQ ID NO: 335 - DNA, 70 bp, Homo sapiens
agaatacagttgtctagccaagccatcaagtgtctgaaattcaatattggtttatgcaaatacagcaaac

SEQ ID NO: 336 - DNA, 70 bp, Homo sapiens
cggctctggttgttggcagctttggggctgtttttgagcttctcattgtgtagaatttctagatcccccg

SEQ ID NO: 337 - DNA, 70 bp, Homo sapiens
tggtggcataattggagccttgctgggcactcctgtaggaggcctgctgatggcatttcagaagtactct

SEQ ID NO: 338 - DNA, 70 bp, Homo sapiens
atacggtgttttctgtccctcctactttccttcacaccagacagcccctcatgtctccaggacaggacag

SEQ ID NO: 339 - DNA, 70 bp, Homo sapiens
cccttatctgctaccctgaatcacctgtcctggtcttgctgtgtgatgggaacatgcttgtaaactgcgt

SEQ ID NO: 340 - DNA, 70 bp, Homo sapiens
cctagcgcgcggggggcgccccccagcccggaggctggctttgctacagctgaccactccggtcaggaga

SEQ ID NO: 341 - DNA, 70 bp, Homo sapiens
gtgaagtgttgcaggttgtgaactctgtagacatctttattgcttggctaagagtagatttaataaatgt

SEQ ID NO: 342 - DNA, 70 bp, Homo sapiens
ccccatagtcaggtgtaccagccagccaaaccaacaccacttcctagaaaaagatcagaagctagtcctc

SEQ ID NO: 343 - DNA, 70 bp, Homo sapiens
gttgctgccatcgtaaactgacacagtgtttataacgtgtacatacattaacttattacctcattttgtt

SEQ ID NO: 344 - DNA, 70 bp, Homo sapiens
ggggaccagcagataaatcccacccttccttgagctgtcgctgtactctgaagttcagccagctcagatt

SEQ ID NO: 345 - DNA, 70 bp, Homo sapiens
gagaaggacaaaatcaccaccaggacactgaaggcccgaatggactaaccctgttcccagagcccacttt

SEQ ID NO: 346 - DNA, 70 bp, Homo sapiens
atctatgatgacgattttttccaaaacctagatggcgtggccaatgccctggacaacgtggatgcccgca

SEQ ID NO: 347 - DNA, 70 bp, Homo sapiens
gcttggagtgaaagtgactctcaggtggtggggtggggaatgtgaataaacatgatttcttgccgggcaa

SEQ ID NO: 348 - DNA, 70 bp, Homo sapiens
gcgtttaaaataaaatatgcaacaaaatggatgacttagtggagatggaagcccattaattgggttcccc

SEQ ID NO: 349 - DNA, 70 bp, Homo sapiens
cactccctaatcccctacccctgtctccccttcaaggacttctcccttgtggttttgtaaagtgcaaact

SEQ ID NO: 350 - DNA, 70 bp, Homo sapiens
gtgaatttttgcacattctacacacagtgcctgtaaatctcatttgtattttcagtttgcccttaatttt

SEQ ID NO: 351 - DNA, 70 bp, Homo sapiens
tttttactccccttcagccccccggctgatgccatctctggttctggacaattatcaaatatatcagtgg

SEQ ID NO: 352 - DNA, 70 bp, Homo sapiens
ggatatagaccacgattccgcaggggccctcctcgccaaagacagcctagagaggacggcaatgaagaag

SEQ ID NO: 353 - DNA, 70 bp, Homo sapiens
caggagggcagtggtggagctggacctgcctgctgcagtcacgtgtaaacaggattattattagtgtttt

SEQ ID NO: 354 - DNA, 70 bp, Homo sapiens
tatttgacagtgtaggaaattgtctattcctgatataattactgtagtactcttgcttaaggcaagagtt

SEQ ID NO: 355 - DNA, 70 bp, Homo sapiens
cgaaggagttgcggttgctccatgttctgacttagggcaatttgattctgcacttggggtctgtctgtac

SEQ ID NO: 356 - DNA, 70 bp, Homo sapiens
cattatgacctgctagagaagaacattaacattgttcgcaaacgactgaaccggccgctgaccctctcgg

SEQ ID NO: 357 - DNA, 70 bp, Homo sapiens
taatttgtaagttatgttagcgggatcctcaaggccttgctttgccccgtggagacgcttgctcggatga

SEQ ID NO: 358 - DNA, 70 bp, Homo sapiens
gcacagatgaaactgagctgggactggaaaggacagcccttgacctgggttctgggtataatttgcactt

SEQ ID NO: 359 - DNA, 70 bp, Homo sapiens
gagacagagtaatttgcagtttgtttgatttatacttttgtttatctacaacccaataacagacatgagg

SEQ ID NO: 360 - DNA, 70 bp, Homo sapiens
ctggggaagcatttgactatctggaacttgtgtgtgcctcctcaggtatggcagtgactcacctggtttt

SEQ ID NO: 361 - DNA, 70 bp, Homo sapiens
gccaaggggccagctgcccctcatttatcactctgaccttcacagggacagatctgatttatttattttg

SEQ ID NO: 362 - DNA, 70 bp, Homo sapiens
gtgggagcagcagagatgtccagggtacagatgcaagtcttgatgaggaacttgatcgagtcaagatgag

SEQ ID NO: 363 - DNA, 70 bp, Homo sapiens
taaaggcccgggagcggctagagctctgtgatgagcgtgtatcctctcgatcacatacagaagaggattg

SEQ ID NO: 364 - DNA, 70 bp, Homo sapiens; identical to SEQ ID NO: 128

SEQ ID NO: 365 - DNA, 70 bp, Homo sapiens
taaaaacaccttgggggcaggcaggggcatttaaaaatgtaggacctatcgtccagactcacagagtggg

SEQ ID NO: 366 - DNA, 70 bp, Homo sapiens
gtggctttccttactgcgaagaatgctaagacccctcagcaggaggagacaacttactaccaaacagcac

SEQ ID NO: 367 - DNA, 70 bp, Homo sapiens; identical to SEQ ID NO: 36

SEQ ID NO: 368 - DNA, 70 bp, Homo sapiens
tcccttctgggttccgaggcccaagcccttggcagtgtttgtgagtggaagggaggtcacgctatcgtcc

SEQ ID NO: 369 - DNA, 70 bp, Homo sapiens
ttatttcccttccacagtgtggtttcttcctctgcggtaaaggacttggtctgttctaccccctgctcca

SEQ ID NO: 370 - DNA, 70 bp, Homo sapiens
gagcattcatcgtgaggggtctttgtcctctgtactgtctctctccttgcccctaacccaaaaagcttca

SEQ ID NO: 371 - DNA, 70 bp, Homo sapiens
ttcatcaagaaccacgcctttcgcctgctgaagccggggggcgtcctcacctactgcaacctcacctcct

SEQ ID NO: 372 - DNA, 70 bp, Homo sapiens
ctgtgaaaataccccctttctccattagtggcatgctcattcagctcttatctttatattccagtaagtt

SEQ ID NO: 373 - DNA, 70 bp, Homo sapiens
cgtccacggactctccgttattttaggaggtccctggccaaagatttatttctcttgacaaccaagggcc

SEQ ID NO: 374 - DNA, 70 bp, Homo sapiens
cgatgagaaggtttactacactgcaggctacaacagtcctgtcaaattgcttaatagaaataatgaagtg

SEQ ID NO: 375 - DNA, 70 bp, Homo sapiens
tgggtgatctctttgctgaattaatgagttcttaacatgtggacccaactgcctgtgtgagatctgtgtc

SEQ ID NO: 376 - DNA, 70 bp, Homo sapiens
ctcacagcggcccgcgggccgggcgtcatgggcggcctcttctggcgctccgcgctgcgggggctgcgct

SEQ ID NO: 377 - DNA, 70 bp, Homo sapiens
tgatcccgcacggcacatcactggggagaagctcggagagctgtataagagctttatcaagaactatcct

SEQ ID NO: 378 - DNA, 70 bp, Homo sapiens
aatacacatttgaaaatttccagtatcaatctagagcgcaaataaatcacagtattgctatgcagaatgg

SEQ ID NO: 379 - DNA, 70 bp, Homo sapiens
ctcatccacagaaagggaggatgggcgatgacagttgtttctatgccttctgacccagtttcccagttta

SEQ ID NO: 380 - DNA, 70 bp, Homo sapiens
acgtctggtaggaagattgttagtgcctcaagttacacctgtgcagcttgggtctgagttttgatagaac

SEQ ID NO: 381 - DNA, 70 bp, Homo sapiens
gaatgtttaggggcctgtgtgaacgcaccaatggttcaaataaatgacaattactatgaggatttgacag

SEQ ID NO: 382 - DNA, 70 bp, Homo sapiens
ggggctgttaagtctgaccatacatcactgtgatagaatgtgggctttttcaagggtgaagatacaagtc

SEQ ID NO: 383 - DNA, 70 bp, Homo sapiens
cgcgctgctccgccgcccgggacttggccgcctcgtccgccacgcccgtgcctatgccgaggccgccgcc

SEQ ID NO: 384 - DNA, 70 bp, Homo sapiens
ctttgttgggaggcggtttgggagaacacatttctaatttgaatgaaatgaaatctattttcagtgaaaa

SEQ ID NO: 385 - DNA, 70 bp, Homo sapiens
ggtgacctctgccccagataggtggtgccagtggcttattaattccgatactagtttgctttgctgacca

SEQ ID NO: 386 - DNA, 70 bp, Homo sapiens
gtttttaaaatcagtactttttaatggaaacaacttgaccaaaaatttgtcacagaattttgagacccat

SEQ ID NO: 387 - DNA, 70 bp, Homo sapiens
gcccctggcttcaccctgtcaggccagctccactccaggactgaataaaggtctttgacagctctaaaaa

SEQ ID NO: 388 - DNA, 70 bp, Homo sapiens
attggcagatcaagcgccagaatggagatgatcccttgctgacttaccggttcccaccaaagttcaccct

SEQ ID NO: 389 - DNA, 70 bp, Homo sapiens
gccaggaggccctgggttccattcctaactctgcctcaaactgtacatttggataagccctagtagttcc

SEQ ID NO: 390 - DNA, 70 bp, Homo sapiens
ttgtggacttcctcattggctccggcctcaagaccatgtccatcgtgagttacaaccacctgggcaacaa

SEQ ID NO: 391 - DNA, 70 bp, Homo sapiens
tgttagagatgctatttgatacaactgtggccatgactgaggaaaggagctcacgcccagagactgggct

SEQ ID NO: 392 - DNA, 70 bp, Homo sapiens
acaaagtgaaaaacagccttttgagtctttctgatacctgagtttttatgcttataatttttgttctttg

SEQ ID NO: 393 - DNA, 70 bp, Homo sapiens
ccgcaatgttggtttcactgagagctgcctcctggtctcttcaccactgtagttctctcatttccaaacc

SEQ ID NO: 394 - DNA, 70 bp, Homo sapiens
gggaggaagcatgtgttctgtgaggttgttcggctatgtccaagtgtcgtttactaatgtacccctgctg

SEQ ID NO: 395 - DNA, 70 bp, Homo sapiens
caaggaaggggtagtaattggcccactctcttcttactggaggctatttaaataaaatgtaagacttcaa

SEQ ID NO: 396 - DNA, 70 bp, Homo sapiens
gttggtgaggtaacatacgtggagctcttaatggacgctgaaggaaagtcaaggggatgtgctgttgttg

SEQ ID NO: 397 - DNA, 70 bp, Homo sapiens
gaaagcacctgctccaaaggcatctggcaagaaagcataagtggcaatcataaaaagtaataaaggttct

SEQ ID NO: 398 - DNA, 70 bp, Homo sapiens
tgcttgtgaacgtgctaagcgtaccctctcttccagcacccaggccagtattgagatcgattctctctat

SEQ ID NO: 399 - DNA, 70 bp, Homo sapiens
ctgccttgttttgcgacattgtcccattcacacagatattttgggataataaaggaaaataagctacaaa

SEQ ID NO: 400 - DNA, 70 bp, Homo sapiens
gatatttaaagttttggcagtaaaatactctgtttttaagtatgaatgtatttcattcatatttcctctc

SEQ ID NO: 401 - DNA, 70 bp, Homo sapiens
tggttgattttgtactttggaactgtaccttggatggttttgtttattaaaagagaaacctgaaccaaaa

SEQ ID NO: 402 - DNA, 70 bp, Homo sapiens
ggaggcagaaccagcaacaactctgggcgtgcctgtgtctgcacatgtggatgtacatatgtctgtatat

SEQ ID NO: 403 - DNA, 70 bp, Homo sapiens
aggcggcgagcggggcccggcgccgaccctgagtgcagcctgacccgccctcgcgcgcgcgccctccccg

SEQ ID NO: 404 - DNA, 70 bp, Homo sapiens
gtgaaaagcctaaatgacatcacagcaaaagagaggttctctcccctcactaccaacctgatcaatttgc

SEQ ID NO: 405 - DNA, 70 bp, Homo sapiens
cgccacccttgacgcttgcagcttcggagtcacgggtttgaaacttcaaggggccacgtgcaacaacaac

SEQ ID NO: 406 - DNA, 70 bp, Homo sapiens
gcccagggaagacacatgattaatgatttagctccctccatacctcgaacatcagttgggatccctcctc

SEQ ID NO: 407 - DNA, 70 bp, Homo sapiens
cgattccactggtggtagtttgctagtgcttctaaaagttgctccctagcactgagaggtgtgggtaggt

SEQ ID NO: 408 - DNA, 70 bp, Homo sapiens
atgggccgacctggctgggactcgtgaatctggagaagagctggagaatggatagtattgtctgtatttg

SEQ ID NO: 409 - DNA, 70 bp, Homo sapiens
gaggacccctacacatcttttgtgaagttgctacctctgaatgattgccgatatgctttgtacgatgcca

SEQ ID NO: 410 - DNA, 70 bp, Homo sapiens
gcgagcgcgcctgcgcgctgggtgattttttcacgtgtcgccagggccggactgcgagtctctttgcggc

SEQ ID NO: 411 - DNA, 70 bp, Homo sapiens
gactacaaatggacgagagaggcggccgtccattagttagcggctccggagcaacgcagccgttgtcctt

SEQ ID NO: 412 - DNA, 70 bp, Homo sapiens; identical to SEQ ID NO: 286

SEQ ID NO: 413 - DNA, 70 bp, Homo sapiens
tttaggctggaagcgccttagaggagccatttttccaggtggggccccaggcagaggctccgacagggag

SEQ ID NO: 414 - DNA, 70 bp, Homo sapiens
cactaccgtggagatcccaactggtttatgaagaaagcgcaggagcataagagggaattcacagagagcc

SEQ ID NO: 415 - DNA, 70 bp, Homo sapiens
cgcttaaatcatgtgaaagggttgctgctgtcagccttgcccactgtgacttcaaacccaaggaggaact

SEQ ID NO: 416 - DNA, 70 bp, Homo sapiens
gagttcgagaccagcctgagcaacatggcgaaaccccgtctctactaaaaatacaaaaatcacccgggtg

SEQ ID NO: 417 - DNA, 70 bp, Homo sapiens
tgaggatggcttgacccgagtcggcttccgcacagtgttgctgagaatacgagaacagtggaaacagaac

SEQ ID NO: 418 - DNA, 70 bp, Homo sapiens
atgttgggcgagtcactgcgtctcgggcattggtgtcctgtcagtaaagagataataatggctgtacctc

SEQ ID NO: 419 - DNA, 70 bp, Homo sapiens
gtgcacgtgtgaagccccctcactcttccgctagggataaagcagatgtggatgccctttaagagatatt

SEQ ID NO: 420 - DNA, 70 bp, Homo sapiens
caggaacctgcttcactgtattaactagtccatgggctgagaccggggcatctcttttcttcatactgca

SEQ ID NO: 421 - DNA, 70 bp, Homo sapiens
cagcatacccccgattccgctacgaccaactcatacacctcctatgaaaaaacttcctaccactcaccct

SEQ ID NO: 422 - DNA, 70 bp, Homo sapiens
gctgcctgccctcctcctctcacccgatgtccaggtgggattttaaagtctgcattggttataataacag

SEQ ID NO: 423 - DNA, 70 bp, Homo sapiens
ataaggtttccagtaagcgggagggcagatccaactcagaaccatgcagataaggagcctctggcaaatg

SEQ ID NO: 424 - DNA, 70 bp, Homo sapiens
ctagttattaagcccagcatgcattagctctttttcctgatgctctccctcccttcatcatccgccctcc

SEQ ID NO: 425 - DNA, 70 bp, Homo sapiens
ctacttctaagtctgaatccagtcagaaataagattttttgagtaacaaataaataagatcagactccaa

SEQ ID NO: 426 - DNA, 70 bp, Homo sapiens
cccacgcgcacttacacgagaagacattcatggctttgggcagaaggattgtgcagattgtcaactccaa

SEQ ID NO: 427 - DNA, 70 bp, Homo sapiens
gaacccctgtggcgcaggactggcctgtgtctgttattttggttgtaaatcattctcctgtggaattggc

SEQ ID NO: 428 - DNA, 70 bp, Homo sapiens
cctgaattcactcgggtatattgattggctggatgatcttggtgccgcccacttgacgtttccagaagag

SEQ ID NO: 429 - DNA, 70 bp, Homo sapiens
gcacaaaggaggctttttctgtgctttgacattctagcacttcagggatgagagggagggagaatcctgg

SEQ ID NO: 430 - DNA, 70 bp, Homo sapiens
gcatccacaccaagagggtgttgtgatgaggtgccggtgtgcaaagggaactttagtttttccactggtt

SEQ ID NO: 431 - DNA, 70 bp, Homo sapiens
gtgtgaaacttgctctactctctgaaatgattcaaatacactaattttccatactttatacttttgttag

SEQ ID NO: 432 - DNA, 70 bp, Homo sapiens
taagcgctgacgcatgcgcatagctaaccgcacccggttcagctcgcctttcttggccagaggcgccggt

SEQ ID NO: 433 - DNA, 70 bp, Homo sapiens
atactttggacttcctctcgccaaagaccttccagcagattctggagtatgcatatacagccacgctgca

SEQ ID NO: 434 - DNA, 70 bp, Homo sapiens
tgtacacttgacaagtgcttactcagcaagtcccagacccacggccttttatctcccaagactggctttg

SEQ ID NO: 435 - DNA, 70 bp, Homo sapiens
gcgccgcccattggtcccgagcgcgatgacttggcgggcggagcaggaaggaaaccgctcccgagcacgg

SEQ ID NO: 436 - DNA, 70 bp, Homo sapiens
cgtggccgcacatcctacagttggaaatccatccagaggccatgttccaataaacaggaggtcgtgtaaa

SEQ ID NO: 437 - DNA, 70 bp, Homo sapiens
tctacgccccagggctgtcgccagacactatcatggagtgtgcaatgggggaccgcggcatgcagctcat

SEQ ID NO: 438 - DNA, 70 bp, Homo sapiens
ccttaagtctaataaggtcatggctgagtctctcagagtgtggacctgcccccttctactctgggcggtt

SEQ ID NO: 439 - DNA, 70 bp, Homo sapiens; identical to SEQ ID NO: 104

SEQ ID NO: 440 - DNA, 70 bp, Homo sapiens
ggcgggggccttggggcagtccgagggtgcggtgaagaggtgacggagggctggctatgggcggccggcc

SEQ ID NO: 441 - DNA, 70 bp, Homo sapiens
gtggtggcaggtgtttaatgacgaccttaccaagccaatcattgataatattgtgtctgatctcattcag

SEQ ID NO: 442 - DNA, 70 bp, Homo sapiens
caaccctgacccgtttgctacatctttttttctatgaaatatgtgaatggcaataaattcatctagacta

SEQ ID NO: 443 - DNA, 70 bp, Homo sapiens
actttgcagtggatcctgaccagccgctgagcgccaagaggaaccccattgacgtggaccccttcaccta

SEQ ID NO: 444 - DNA, 70 bp, Homo sapiens
cttcttcttctctcccagctgaacccgaggctaaagaagatgaggcaagagaaaatgtaccccaaggtga

SEQ ID NO: 445 - DNA, 70 bp, Homo sapiens
cacatggctgggctgacagcatcccctacacccccttcttcaagcataattacttactgactttcctcca

SEQ ID NO: 446 - DNA, 70 bp, Homo sapiens
ccaccctggagccaagggtctttcacatcacctatccctacatacataccaaatggaaaagtggccatcc

SEQ ID NO: 447 - DNA, 70 bp, Homo sapiens
ttaagactttccaaagatgaggtccctggtttttcatggcaacttgatcagtaaggatttcacctctgtt

SEQ ID NO: 448 - DNA, 70 bp, Homo sapiens
aggagcagtaaacatagccaaggcctaagggatcaaggaaaccaagagcaggatccaaatatttccaatg

SEQ ID NO: 449 - DNA, 70 bp, Homo sapiens
agaagggccccaatgccaactcttaagtcttttgtaattctggctttctctaataaaaaagccacttagt

SEQ ID NO: 450 - DNA, 70 bp, Homo sapiens
acattccagatggctatcctgcttcagtacaacacggaagatgcctacactgtgcagcagctgaccgaca

SEQ ID NO: 451 - DNA, 70 bp, Homo sapiens
ggggcatcagagtcttggctgggctgaatctgctgcttgttggttcagtgtttcttatgaacaagagcca

SEQ ID NO: 452 - DNA, 70 bp, Homo sapiens
gagagttcgatatgattcttgggaaactagagaatgacggaagtagaaagcctggagtcatagataagtt

SEQ ID NO: 453 - DNA, 70 bp, Homo sapiens
gagagttgctgcctttgatagacccatgctgaccacagcctgatattccagaacctggaacagggacttt

SEQ ID NO: 454 - DNA, 70 bp, Homo sapiens
gtctgagcaaggggtgtacacctgcacagcacagggcatttggaagaatgaacagaagggagagaagatt

SEQ ID NO: 455 - DNA, 70 bp, Homo sapiens
agagaccgctggcagcaccagtattcccaagaggaagaagtctacacccaaggaggaaacagttaatgac

SEQ ID NO: 456 - DNA, 70 bp, Homo sapiens
ctaagactcgcggcaggttctctttgagtcaatagcttgtcttcgtccatctgttgacaaatgacagatc

SEQ ID NO: 457 - DNA, 70 bp, Homo sapiens
gccagatagctaggtttctggttcccccacagtaggtgttttcacataagattagggtccttttggaaag

SEQ ID NO: 458 - DNA, 70 bp, Homo sapiens
aagcacgttgcccaaggttgcacagcaagaaaagggagaagttgagattcaaacccaggctgtctagctc

SEQ ID NO: 459 - DNA, 70 bp, Homo sapiens
ctgcaaagaggccaacacactagaaatcagaaatcttgactcctagcccaccgtcccctaaaacatgggc

SEQ ID NO: 460 - DNA, 70 bp, Homo sapiens
cgcggtttggtttgcagcgactggcatactatgtggatgtgacagtggcgtttgtaatgagagcactttc

SEQ ID NO: 461 - DNA, 70 bp, Homo sapiens
tcggactcctgcctcactcatttacaccaaccacccaactatctataaacctagccatggccatcccctt

SEQ ID NO: 462 - DNA, 70 bp, Homo sapiens
aggagcagcccatggagacgacgggcgccaccgagaacggacatgaggccgtccccgaaggcgagtcgcc

SEQ ID NO: 463 - DNA, 70 bp, Homo sapiens
gcttcttgctgccgccatatgaagaaggacgtgttcgcttccccttcctccatgattgtaagtttcctga

SEQ ID NO: 464 - DNA, 70 bp, Homo sapiens
catgttaaaatggggaaggatgatagctacatgtatgccggtcctactcacgcgacacccgtgtgctcaa

SEQ ID NO: 465 - DNA, 70 bp, Homo sapiens
acatgaccccagcaactgtggtggtatctagaggtgaaacaggcaagtgaaatggacacctctgctgtga

SEQ ID NO: 466 - DNA, 70 bp, Homo sapiens
gcccctggcaaatgcacacacctcatgctagcctcacgaaactggaataagccttcgaaaagaaattgtc

SEQ ID NO: 467 - DNA, 70 bp, Homo sapiens
gtggttgatggcgccttcaaagaggtgaagctgtcggactacaaagggaagtacgtggtcctctttttct

SEQ ID NO: 468 - DNA, 70 bp, Homo sapiens
tccttcctagtaatactttgcctttttcactgtgtatggaatgaaacatgtaaagctgtcacaatcaatg

SEQ ID NO: 469 - DNA, 70 bp, Homo sapiens
gcaagactcttacgccccacactgcaatttggtcttgttgccgtatccatttatgtgggcctttctcgag

SEQ ID NO: 470 - DNA, 70 bp, Homo sapiens
ccacagaagacacgtgtttttgtatctttaaagacttgatgaataaacactttttctggtcaatgtcaaa

SEQ ID NO: 471 - DNA, 70 bp, Homo sapiens
gcagctttgaactagggctggggttgtgggtgcctcttctgaaaggtctaaccattattggataactggc

SEQ ID NO: 472 - DNA, 70 bp, Homo sapiens
cccaggctttgtcccaggctttctggtgtgtgccctcctggtaacagtgaaattgaagctacttactcat

SEQ ID NO: 473 - DNA, 70 bp, Homo sapiens
caggtgcctagtcttgagtgaattgttagatgtgcactgaactcgggatgttggggattggagagagaga

SEQ ID NO: 474 - DNA, 70 bp, Homo sapiens
aaaagtattttgtggtgaccataagaatgtccctccccaaacaagtaaacttgtgaaagtttaatttgga

SEQ ID NO: 475 - DNA, 70 bp, Homo sapiens
atgatcctgttagctcttccagctctccaggcgccaacaaccatatggtctcggtaacgactgctcccca

SEQ ID NO: 476 - DNA, 70 bp, Homo sapiens
gagaatacaagatattatgtataaaatgtaacactgatgataggttaataaagatgattgaatccaaaaa

SEQ ID NO: 477 - DNA, 70 bp, Homo sapiens
taccccttccactgctcactttgtggatggtagcatgagctgtctaccaagaagaaacctgctgctctct

SEQ ID NO: 478 - DNA, 70 bp, Homo sapiens
caactggatgaaaaggaaaaggatttggtgggcctggctcagatcgcagaggtcctcgagatgttcgatt

SEQ ID NO: 479 - DNA, 70 bp, Homo sapiens
ctaatccccttgatgagctttcacgaagtctcacggcttctctagggactccatggtcttcagagtcgtt

SEQ ID NO: 480 - DNA, 70 bp, Homo sapiens
agatgggatagtttactgactagttggagcatttgtaagcacatggtgaaatcagcccctgcccaccaaa

SEQ ID NO: 481 - DNA, 70 bp, Homo sapiens
cctgggattctttttctagggatgtaatacatatatttacaaataaaatgcctcatggactctggtgaaa

SEQ ID NO: 482 - DNA, 70 bp, Homo sapiens
tttaatcgctttgaataaatactcccttaagtagttaaatataggaggagaaagaatacatcggttgtta

SEQ ID NO: 483 - DNA, 70 bp, Homo sapiens
ggcaatgcctacccccagcgttatttttggggagggagggctgtgcatagggacatattctttagaatct

SEQ ID NO: 484 - DNA, 70 bp, Homo sapiens
tggaataaaaggagagaagggtttccccggattccctggactggacatgccgggccctaaaggagataaa

SEQ ID NO: 485 - DNA, 70 bp, Homo sapiens
ggccaaccgagcgccatgaaccagatagagcccggcgtgcagtacaactacgtgtacgacgaggatgagt

SEQ ID NO: 486 - DNA, 70 bp, Homo sapiens
gccaaagtgctcagagaccttctatgacacattagtgtcacatggttgcgtgtccagccgaagcagtgta

SEQ ID NO: 487 - DNA, 70 bp, Homo sapiens
atacaaaagtggcacatgcctgtaatgccagctactggggaggctgaggtaggagaattgcttgaacctg

SEQ ID NO: 488 - DNA, 70 bp, Homo sapiens
ccggcagttcttgggtcaaatgacacaattaaaccaactcctgggagaggtgaaggaccttctgagacag

SEQ ID NO: 489 - DNA, 70 bp, Homo sapiens
gtggctggcccggcctccacagcaccccaccccatatcttctttccatttatttcgtaccaaaaacaatt

SEQ ID NO: 490 - DNA, 70 bp, Homo sapiens
cattttttgtaatttttgtaaaacaaaagtaccaatctgttttgtaaataaaaatcatcctaaaattcga

SEQ ID NO: 491 - DNA, 70 bp, Homo sapiens
tgatctttctggctccactcagtgtctaaggcaccctgcttcctttgcttgcatcccacagactatttcc

SEQ ID NO: 492 - DNA, 70 bp, Homo sapiens
tcataactggcttctgcttgtcatccacacaacaccaggacttaagacaaatgggactgatgtcatcttg

SEQ ID NO: 493 - DNA, 70 bp, Homo sapiens
agccaggatttccctcagtgcaacaccattgagaatacaggaactaaacagtccacctgtagtccagggg

SEQ ID NO: 494 - DNA, 70 bp, Homo sapiens
cgtagactcgctcatctcgcctgggtttgtccgcatgttgtaatcgtgcaaataaacgctcactccgaat

SEQ ID NO: 495 - DNA, 70 bp, Homo sapiens
gacactggcccctctcaggtcagaagacatgcctggagggatgtctggctgcaaagactatttttatcct

SEQ ID NO: 496 - DNA, 70 bp, Homo sapiens
tttgcccagcacgccaacgccttccaccagtggatccaagagaccaggacatacctcctcgatgggtcct

SEQ ID NO: 497 - DNA, 70 bp, Homo sapiens
cacaacatgaaagaaatggtgctacccagctcaagcctgggcctttgaatccggacacaaaaccctctag

SEQ ID NO: 498 - DNA, 70 bp, Homo sapiens
atccccatgcccttgacctcttctggcattctcctgtgctctgacaaactgagccagccttttagatcta

SEQ ID NO: 499 - DNA, 70 bp, Homo sapiens
aagtttccgaccctggcttataggcaccacacctcatgtactcctcatggcttggatctctgtattcagc

SEQ ID NO: 500 - DNA, 70 bp, Homo sapiens
aaggtctgacgccacctcaaggtgacagctcatctccagcacagcacaggcgtgtgcacacagaggtgtt

SEQ ID NO: 501 - DNA, 70 bp, Homo sapiens
cggagcagagacaggccctcggggtggaggtctttggtttcataagagcctgagagagatttttctaaga

SEQ ID NO: 502 - DNA, 70 bp, Homo sapiens
ataagtcacattggttccatggccacaaaccattcagatcagccacttgctgaccctggttcttaaggac

SEQ ID NO: 503 - DNA, 70 bp, Homo sapiens
ctacttcggagtctatgatactgccaaggggatgctgcctgaccccaagaacgtgcacatttttgtgagc

SEQ ID NO: 504 - DNA, 70 bp, Homo sapiens
actgttgcttgctggtcgcagactccctgacccctccctcacccctccctaacctcggtgccaccggatt

SEQ ID NO: 505 - DNA, 70 bp, Homo sapiens
gacagggccagtgcagtttggtgtgtcctccgcctttccaggagaagaacctgaagaactatttttcgtt

SEQ ID NO: 506 - DNA, 70 bp, Homo sapiens
ggtcagggactgaatcttgcccgtttatgtatgctccatgtctagcccatcatcctgcttggagcaagta

SEQ ID NO: 507 - DNA, 70 bp, Homo sapiens
gggagagtgccgggcggtcggcgggtcagggcagcccggggcctgacgccatgtcccggaacctgcgcac

SEQ ID NO: 508 - DNA, 70 bp, Homo sapiens
tgctctaagggaccttggagacaggcctttcaggtggatgttcatgtttctgaccttgcactaccccaat

SEQ ID NO: 509 - DNA, 70 bp, Homo sapiens
attcgccgttcgaaagcagggactaaaagccccacttcgtcttacgttccgaaaggaaggcgtctgttga

SEQ ID NO: 510 - DNA, 70 bp, Homo sapiens
ggtggagttgttagtgtcctatggcaacaccttctttgtggttctcattgtcatccttgtgctgttggtc

SEQ ID NO: 511 - DNA, 70 bp, Homo sapiens
ttgcctcatcaccttgtccaaatgagctagacctccctgtcccggagggaaaaacatctgaaaagcagac

SEQ ID NO: 512 - DNA, 70 bp, Homo sapiens
tttttaagctcaagcaaatgtttggtaatgcagacatgaatacatttcacaccttcaaatttgaagatcc

SEQ ID NO: 513 - DNA, 70 bp, Homo sapiens; identical to SEQ ID NO: 76

SEQ ID NO: 514 - DNA, 70 bp, Homo sapiens
gaactgtggccacctagaaaggggcccattcagcctcgtctctttacagaagtagttttgttcatgaaat

SEQ ID NO: 515 - DNA, 70 bp, Homo sapiens
actccaagaagtacattgccttctgcatcagcatcttcacggccatcctggtgaccatcgtgatcctcta

SEQ ID NO: 516 - DNA, 70 bp, Homo sapiens
aagacctgaaccagagatccatcatggagagcccagccaacagtattgagatgcttctgtccaacttcgg

SEQ ID NO: 517 - DNA, 70 bp, Homo sapiens
tatttttcttaacatgttagtacttctacgactttggagccactgatgggtccactcatggcctcagctg

SEQ ID NO: 518 - DNA, 70 bp, Homo sapiens
ggtcagcaaaggaaagtggaagttggattctgaaagatcgaggtgcccacaggaattttatggtcgtcgg

SEQ ID NO: 519 - DNA, 70 bp, Homo sapiens
agcacacccgtctatgtagcaaaatagtgggaagatttataggtagaggcgacaaacctaccgagcctgg

SEQ ID NO: 520 - DNA, 70 bp, Homo sapiens
aaggaaaaccggccccagaaacaggggtgtgctttcccaccaataaaaggccgtggaacccgagggcttt

SEQ ID NO: 521 - DNA, 70 bp, Homo sapiens
gggatatagggtcgaagccgcactcgtaaggggtggatttttctatgtagccgttgagttgtggtagtca

SEQ ID NO: 522 - DNA, 70 bp, Homo sapiens
gctcctttgttttacagagcagggtcacttgatttgctagctggtggcagaattggcaccattacccagg

SEQ ID NO: 523 - DNA, 70 bp, Homo sapiens
tgaacaaaagaagccacgaggtggaacaaggtctctgtcagtcacaggcacccctgagaaccgggaacat

SEQ ID NO: 524 - DNA, 70 bp, Homo sapiens
gctactgagggtctaagtccgggcagccgaagagtgtggtaggtaacggtcctcagcgcaagggtcattt

SEQ ID NO: 525 - DNA, 70 bp, Homo sapiens
tctacaaagggttcatgccctcctttctccgcttgggttcctggaacgtggtgatgttcgtcacctatga

SEQ ID NO: 526 - DNA, 70 bp, Homo sapiens
tttatccccagaccaggcatcacctatgagccacccaactataaggccctggacttctccgaggccccaa

SEQ ID NO: 527 - DNA, 70 bp, Homo sapiens
atgccgtcggaaatggtgaagggagactcgaagtactctgaggcttgtaggagggtaaaatagagaccca

SEQ ID NO: 528 - DNA, 70 bp, Homo sapiens
tttttaagtagcctcctttccactatttagtaattggctgtgagctgggctgggggagaaatggggcggg

SEQ ID NO: 529 - DNA, 70 bp, Homo sapiens
tttttgagacagagttttgctctcgttgcccaagcttgagtgtaatggcatggtcttggctcactgcact

SEQ ID NO: 530 - DNA, 70 bp, Homo sapiens
  misc_feature (8)..(9): n is a, c, g, or t
accctggnnagatagacttccctgtttccaaggggcgtgggactttctaccacgtccatcaactcgtggc

SEQ ID NO: 531 - DNA, 70 bp, Homo sapiens
atagtgtttggcttattttccatcccagttctgggaggtcttttaagtctccttcctttggttgccccac

SEQ ID NO: 532 - DNA, 70 bp, Homo sapiens
aactatttgcgcaatctgtgggtctgtggattcacggggctttctgtgtgggtgctgcagttgcttttgt

SEQ ID NO: 533 - DNA, 70 bp, Homo sapiens
tgggccttgtgacattgtctacctgtggtcattccttaactgctttggcctcaactttgagctctggatg

SEQ ID NO: 534 - DNA, 70 bp, Homo sapiens
ttttaaaaatccacttatggctgggcacagaagctcacgcctgtaatcccagcactttgggaggctgagg

SEQ ID NO: 535 - DNA, 70 bp, Homo sapiens
aaaccccatctctactaaaaatacaaaaaattagccgggcgtggtagcgggcgcctgtagtcccagctac

SEQ ID NO: 536 - DNA, 70 bp, Homo sapiens
ctggatcttggcctttacattttctatcgtatccgagggttcaacctcgagggtgatggtcttccccgta

SEQ ID NO: 537 - DNA, 70 bp, Homo sapiens
ggacaagaacacagtcaactttggctttgcttggaaagctgcttcagatacataactcccggcccctcct

SEQ ID NO: 538 - DNA, 70 bp, Homo sapiens; identical to SEQ ID NO: 444

SEQ ID NO: 539 - DNA, 70 bp, Homo sapiens
atccaaccctttaagatgagtgccactggttgcccattttacagatgagaaactgggctcacagacacac

SEQ ID NO: 540 - DNA, 70 bp, Homo sapiens
ggacatttgggttggttccaagtctttgctattgtgaatagtgccgcaataaacatacgtgtgcatgtgt

SEQ ID NO: 541 - DNA, 70 bp, Homo sapiens
atcagccgtaagcctagaagcagagcgggatcgaggcgtttttaataattcgagttgggaagacccggat

SEQ ID NO: 542 - DNA, 70 bp, Homo sapiens
gctctagctacttggactattcagggagctgcaaatgccctctctggtgacgtttgggacattgacaatg

SEQ ID NO: 543 - DNA, 70 bp, Homo sapiens
gcggagattcaaggacctaagcttccaggaggagtacagcacactgttccctgcctcggcacagccgtag

SEQ ID NO: 544 - DNA, 70 bp, Homo sapiens
cagtgttcgaatcatcgacaaaaatggcatccatgacctggataacatttccttccccaaacagggctcc

SEQ ID NO: 545 - DNA, 70 bp, Homo sapiens
tatcttgctggtcaaaatatacaagatgtgagcctggaaagccttcggagggcagtgggagtggtacctc

SEQ ID NO: 546 - DNA, 70 bp, Homo sapiens
  misc_feature (10)..(10): n is a, c, g, or t
ggcttctggnaagctgttgnagcccaattgaaccanaaagtttggtggcctatcagntggaccttgtatg

SEQ ID NO: 547 - DNA, 70 bp, Homo sapiens
accttcactgtcagcgcctggaaaacttggctcacgaaaccagggaacgaagaaaaacctccaggggaac

SEQ ID NO: 548 - DNA, 70 bp, Homo sapiens
ccctctggtccagcccctcacgcctcctctcagtctactcaattgtgactgtccctcctgatgtattttt

SEQ ID NO: 549 - DNA, 70 bp, Homo sapiens; identical to SEQ ID NO: 136

SEQ ID NO: 550 - DNA, 70 bp, Homo sapiens
ctctgtgtttcatgtgtcccaggtcccccaaaaaacaggtggtggtggattatacatggctttcagtagt

SEQ ID NO: 551 - DNA, 70 bp, Homo sapiens
agtacctgcacaaccagcacatcctgcacctggacctgaggtccgagaacatgatcatcaccgaatacaa

SEQ ID NO: 552 - DNA, 70 bp, Homo sapiens
  misc_feature (18)..(18): n is a, c, g, or t
caccactctgaacagctncttgatggtgtcattcaagttattgggctttctctcccgctggagcctcagc

SEQ ID NO: 553 - DNA, 70 bp, Homo sapiens
ggacgtgtaaacagacggtaccctactcttgtggcaatcactaagtttcagccaaccaaagacagcgaac

SEQ ID NO: 554 - DNA, 70 bp, Homo sapiens
ratcataagtgagahtcykcccagtyttmtttgtgcttytcttttgggragawttagtaaytgtgccact

SEQ ID NO: 555 - DNA, 70 bp, Homo sapiens
ctacaataagggcaactgcagtctcatatgtccaacatcgagcaacattacggattgtgtagccacctcc

SEQ ID NO: 556 - DNA, 70 bp, Homo sapiens
kgyaccacaggrttgagccgtcgaggggkgagtgctgttattatwtcttaaaaaatctgatgacccgggv

SEQ ID NO: 557 - DNA, 70 bp, Homo sapiens
cactgacagggatcaagtttgtggttctagcagatcctaggcaagctggaatagattctcttctccgaaa

SEQ ID NO: 558 - DNA, 70 bp, Homo sapiens
aaatggacaaggccaggtatagcgaatggctttgctcctgtagagaaccgtcactcggtcagamaarcct

SEQ ID NO: 559 - DNA, 70 bp, Homo sapiens; identical to SEQ ID NO: 527

SEQ ID NO: 560 - DNA, 70 bp, Homo sapiens
atcacctgctgtatgccgatcatctcagaaagggctgtgtagagtagggccctgttctccttaggatgtt

SEQ ID NO: 561 - DNA, 70 bp, Homo sapiens
cgacatcattgttggcgatggtgatgaccacatctgggacattgtagggagtgtctaggtgactctccat

SEQ ID NO: 562 - DNA, 70 bp, Homo sapiens
ctctgtatgagaactccccagagttcacaccttacctggagacaaacctcggacagccaacaattcagag

SEQ ID NO: 563 - DNA, 70 bp, Homo sapiens
agcctggggtgcttcgtgggctcccgctttgtccacggcgagggtctccgctggtacgccggcctgcaga

SEQ ID NO: 564 - DNA, 70 bp, Homo sapiens
tcatcgacatgctcatggagaacatctccaccaagggcctggactgtgacattgacatccagaagacatc

SEQ ID NO: 565 - DNA, 70 bp, Homo sapiens
tgagtcccgggtagttggagcctgtcagtcgccgggtcagtaggtcgcggagtctgcgagaagccactat

SEQ ID NO: 566 - DNA, 70 bp, Homo sapiens
aagttacgcagatcccataaagctacggtcttatccgcagagccggtggctagaataaaatcgcctgtag

SEQ ID NO: 567 - DNA, 70 bp, Homo sapiens
aagattattttttaaatcctgaggactagcattaattgacagctgacccaggtgctacacagaagtggat

SEQ ID NO: 568 - DNA, 70 bp, Homo sapiens
gaagccagactacactgcttacgttgccatgatccctcagtgcataaaggaggaagacaccccttcagat

SEQ ID NO: 569 - DNA, 70 bp, Homo sapiens
acccacaggtcctaaactaccaaacctgcattaaaaatttcggttggggcgacctcggagcagaacccaa

SEQ ID NO: 570 - DNA, 70 bp, Homo sapiens
gcatgaaaaactccaaataagagatcyctcaggattataaaagttgtaaatgcactgtwtkctggsaaaa

SEQ ID NO: 571 - DNA, 70 bp, Homo sapiens
ccttctgcacatctaaacttagatggagttggtcaaatgagggaacatctgggttatgccttttttaaag

SEQ ID NO: 572 - DNA, 70 bp, Homo sapiens
agggtcttctcgtcttgctgtgtcatgcccgcctcttcacgggcaggtcaatttcactggttaaaagtaa

SEQ ID NO: 573 - DNA, 70 bp, Homo sapiens
cctcttccggagatgtagcaaaacgcatggagtgtgtattgttcccagtgacacttcagagagctggtag

SEQ ID NO: 574 - DNA, 70 bp, Homo sapiens
atgtgtaccttggagtcatcctcttggtcttgtattcatattgtgggacagtgggaatagcagcttgtag

SEQ ID NO: 575 - DNA, 70 bp, Homo sapiens
accttttctggcaagactgctctgcatttctgctgccctcatacctcacccagccaacctaccaaacatt

SEQ ID NO: 576 - DNA, 70 bp, Homo sapiens
ccataaagactccgtgtaactgtgtgaacacttgggatttttctcctctgtcccgaggtcgtcgtctgct

SEQ ID NO: 577 - DNA, 70 bp, Homo sapiens
acctgttgttacagggcaggatcggatgatggacactgaagtcctcagcttgctaagttcagttgctctc

SEQ ID NO: 578 - DNA, 70 bp, Homo sapiens
tgtttctaccaacactgcaccttatcccaggaacctgccctagacctccagagaccatattttctctccc

SEQ ID NO: 579 - DNA, 70 bp, Homo sapiens
aacttgaacctaaaaattagcccctcatagtgtagccgccggactttgctcatagctggcaggctggact

SEQ ID NO: 580 - DNA, 70 bp, homo sapiens
gtaggagctcgtcactcttttgacaaaaagggggtgattgtggttgaagtggaggacagagagaagaagg

SEQ ID NO: 581 - DNA, 70 bp, Homo sapiens
gacagtgtgggtatcaagagccaatgtgatccagcgccggggccgggcgggccgctgccagtccggcttt

SEQ ID NO: 582 - DNA, 70 bp, Homo sapiens; identical to SEQ ID NO: 423

SEQ ID NO: 583 - DNA, 70 bp, Homo sapiens
  misc_feature (15)..(15): n is a, c, g, or t
ctgcagtcctcactngagaaaatcactccctctgggagattggaagttgctggaaagaaaacaggtccaa

SEQ ID NO: 584 - DNA, 70 bp, Homo sapiens
aatctggccaaaagagttcgcgctttcccccatggatgttttctaccacaagaatataagtgctgaaaat

SEQ ID NO: 585 - DNA, 70 bp, Homo sapiens
cagagtacttcgagtctcccttcaccatttccgacggcatctacggctcaacattttttgtagccacagg

SEQ ID NO: 586 - DNA, 70 bp, Homo sapiens
cgcccacggacttacatcctcattactattctgcctagcaaactcaaactacgaacgcactcacagtcgc

SEQ ID NO: 587 - DNA, 70 bp, Homo sapiens
tgcgacaggcacgcagcctactaggtgtggcggcgaccctggccccgggttcccgtggctaccgggcgcg

SEQ ID NO: 588 - DNA, 70 bp, Homo sapiens
tgaatggtcagcttgtccacagggtgaatcttgttgtagtcagccgggtcagcgaaggtcagaggcagca

SEQ ID NO: 589 - DNA, 70 bp, Homo sapiens
cccatcatactctttcacccacagcaccaatcctacctccatcgctaaccccactaaaacactcaccaag

SEQ ID NO: 590 - DNA, 70 bp, Homo sapiens
gcacccaatacaggagcagccagattcataaagcaagtcctgagtgacctacaaagagacttagactccc

SEQ ID NO: 591 - DNA, 70 bp, Homo sapiens
agctctctgctctcccagcgcagcgccgccgcccggcccctccagcttcccggaccatggccaacctgga

SEQ ID NO: 592 - DNA, 70 bp, Homo sapiens
ttcagcgtggggcgcccacaatttgcgcgctctctttctgctgctccccagctctcggatacagccgaca

SEQ ID NO: 593 - DNA, 70 bp, Homo sapiens
cccaacccgtcatctactctaccatctttgcaggcacactcatcacagcgctaagctcgcactgattttt

SEQ ID NO: 594 - DNA, 70 bp, Homo sapiens
gagtacaccgactacggcggactaatcttcaactcctacatacttcccccattattcctagaaccaggcg

SEQ ID NO: 595 - DNA, 70 bp, Homo sapiens
ctggagccggagcaccctatgtcgcagtatctgtctttgattcctgcctcatcctattatttatcgcacc

SEQ ID NO: 596 - DNA, 70 bp, Homo sapiens
cttcgaatgtgtggtaggggtggggggcatccatatagtcactccaggtttatggagggttcttctacta

SEQ ID NO: 597 - DNA, 70 bp, Homo sapiens
tctcaacttagtattatgcccacacccacccaagaacagggtttgttaagatggcagagcccggtaatcg

SEQ ID NO: 598 - DNA, 70 bp, Homo sapiens
agcattcctgcacatctgtacccacgccttcttcaagccatactatttatgtgctccggggtcatcatcc

SEQ ID NO: 599 - DNA, 70 bp, Homo sapiens; identical to SEQ ID NO: 593

SEQ ID NO: 600 - DNA, 70 bp, Homo sapiens
ccgccatcttcagcaaaccctgatgaaggctacaaagtaagcgcaagtacccacgtaaagacgttaggtc

SEQ ID NO: 601 - DNA, 70 bp, Homo sapiens
ccgggatcgtcatctactctaccatctttgcaggcacactcatcacagcgctaagctcgcactgattttt

SEQ ID NO: 602 - DNA, 70 bp, Homo sapiens
acttttgaaattcacacattgtgaagcctgccagtccccgccaggtgaagagctcatggtatccaccttc

SEQ ID NO: 603 - DNA, 70 bp, Homo sapiens
ctggtgaagccccagctatcatggcagtgaagggctctggctagatttggatgtcaactgctgagttcta

SEQ ID NO: 604 - DNA, 70 bp, Homo sapiens
cgctggaccggtccggattcccgggatgtccacacaggcagacttgaccttgacagatagtcttcaagat

SEQ ID NO: 605 - DNA, 70 bp, Homo sapiens; identical to SEQ ID NO: 569

SEQ ID NO: 606 - DNA, 70 bp, Homo sapiens
caccctagtaggctcccttcccctactcatcgcactaatttacactcacaacaccctaggctcactaaac

SEQ ID NO: 607 - DNA, 70 bp, Homo sapiens
acagagctccttcaaacttcagaacggcctatgaaggagtcccgtggaaacatctgggaggactttcaag

SEQ ID NO: 608 - DNA, 70 bp, Homo sapiens
tagttagggccctcggccacactcaagttctgctcctccaacagggcctgaaagttttttcggaagcgaa

SEQ ID NO: 609 - DNA, 70 bp, Homo sapiens
tggtcgtgggagggctgaacacacattaccgctacattggcaagaccatggattaccggggaaccatgat

SEQ ID NO: 610 - DNA, 70 bp, Homo sapiens
acgcgtccgctctgacttcttggactacatggggatcaaaggccccaggatgcctctgggcttcacgttc

SEQ ID NO: 611 - DNA, 70 bp, Homo sapiens
acagaatatcctgtagaaaaactaatgagggatgccaaaatctatcagatttatgaaggtacttcacaaa

SEQ ID NO: 612 - DNA, 70 bp, Homo sapiens
cccacgcgtccgagcaagttgaaaatggattgagactgcatggtggcataaatgagaaattgcctgtagc

SEQ ID NO: 613 - DNA, 70 bp, Homo sapiens
caaagtagtgatggattcagtactcctcaaccactctcctaatgattggaacaaaagcaaacaaaaaaga

SEQ ID NO: 614 - DNA, 70 bp, Homo Sapiens
tacccagcacatcccactataccagatgagtggcttctatggcaagggtccctccattaagcagttcatg

SEQ ID NO: 615 - DNA, 70 bp, Homo sapiens
aagaacagtacaaagaacatccgtgtacccagtaccctgactaccgactacctacaacccgtccctgccc

SEQ ID NO: 616 - DNA, 70 bp, Homo sapiens
ccttaccaccaaacataccaaaatgcacctctttcataagtgagttactaagatttctatacctggaata

SEQ ID NO: 617 - DNA, 70 bp, Homo sapiens
cctatttggaccagaaaccctgatgacatcacccaagaggagtatggagaattctacaagagcctcacta

SEQ ID NO: 618 - DNA, 70 bp, Homo sapiens
acgggagaggtactgaggacaaatcagttctctgtgaccagacatgaaaaggttgccaatgggctgttgg

SEQ ID NO: 619 - DNA, 70 bp, Homo sapiens
taccctagccaaccccttaaacacccctccccacatcaagcccgaatgatatttcctattcgcctacaca

SEQ ID NO: 620 - DNA, 70 bp, Homo sapiens
tacagagtcacactcaatcctccgggcaccttccttgaaggagtggctaaggttggacaatacacgttca

SEQ ID NO: 621 - DNA, 70 bp, Homo sapiens
acccttggccataatatgatttatctccacactagcagagaccaaccgaacccccttcgaccttgccgaa

SEQ ID NO: 622 - DNA, 70 bp, Homo sapiens
gcccacttcttaccacaaggcacacctacaccccttatccccatactagttattatcgaaaccatcagcc

SEQ ID NO: 623 - DNA, 70 bp, Homo sapiens
cacttctggttgccaggagacagcaagcaaagccagcaggacatgaagttgctattaaatggacttcgtg

SEQ ID NO: 624 - DNA, 70 bp, Homo sapiens; identical to SEQ ID NO: 214

SEQ ID NO: 625 - DNA, 70 bp, Homo sapiens
tgacctggcctctcccccacaggaacaaaacactgcctccagagtctttaaattctcagttatcaacgcc

SEQ ID NO: 626 - DNA, 70 bp, Homo sapiens
gagaaggtaagcacatttgaggccacctagcctttgcttctctgttcaaatcaattatatttcaaaagct

SEQ ID NO: 627 - DNA, 70 bp, Homo sapiens
ggtgtacactcaaaacctgtccccggcagccagtgctctctgtatagggccataatggaattctgaagaa

SEQ ID NO: 628 - DNA, 70 bp, Homo sapiens
gggacatgcttccccttgtccacctttgcagcctgtttctgtcatgtagtttcaacaagtgctacctttg

SEQ ID NO: 629 - DNA, 70 bp, Homo sapiens
acgctcttcgctgtcgtttgtggtctcgcgcagggcggccccggttctggtgtttggcgtcggaattaaa

SEQ ID NO: 630 - DNA, 70 bp, Homo sapiens
taagagacgacagggaccgaagaggacctccactcagatcagaacgtgaagaagtaagttcttggagacg

SEQ ID NO: 631 - DNA, 70 bp, Homo sapiens
gcacaggctgtggcttgcactccagccgctctagtctctcaggaatttgcttgttacttgtactgtgtaa

SEQ ID NO: 632 - DNA, 70 bp, Homo sapiens
ccaaaccaactctttgccagcagccacaacatgcattgacagcggcacagtgagatataactgatgggct

SEQ ID NO: 633 - DNA, 70 bp, Homo sapiens
tatatattgtgcatcaactctgttggatacgagaacactgtagaagtggacgatttgttctagcaccttt

SEQ ID NO: 634 - DNA, 70 bp, Homo sapiens
tcagaaatgaggtgtaattccccaacccctgcccgcaagagctaagtaggatcttactgtaagttgaagg

SEQ ID NO: 635 - DNA, 70 bp, Homo sapiens; identical to SEQ ID NO: 111

SEQ ID NO: 636 - DNA, 70 bp, Homo sapiens
  misc_feature (36)..(36): n is a, c, g, or t
gcatttcttctatgcacttatcagaaagatcaaagnctttaatactttcactaattttgctactgctatc

SEQ ID NO: 637 - DNA, 70 bp, Homo sapiens
accggcgtcaaagtatttagctgactcgccacactccacggaagcaatatgaaatgatctgctgcagtgc

SEQ ID NO: 638 - DNA, 70 bp, Homo sapiens
ttgagaagtatcctgaggcatggggggttcatagtagaagagcgatggtgagagctaaggtcggggcggt

SEQ ID NO: 639 - DNA, 70 bp, Homo sapiens
tccccctacacctgtgtcagctgcggtgcccgggcaggtacatctaccttcgaggcaggagccctctcat

SEQ ID NO: 640 - DNA, 70 bp, Homo sapiens
tttccatcaattagctcccgcacaagtgtggtctcttgcccgtcccatttctgcaggtgaacaagtttcc

SEQ ID NO: 641 - DNA, 70 bp, Homo sapiens
ggaaatgtgagccctgcattctgaatgagttttaggattatattctgattcactaattctcctttcaacc

SEQ ID NO: 642 - DNA, 70 bp, Homo sapiens
gggagtgtaaaatgcttcagccactttagaaaatagttttgcagtttcttacaacattaaaaatatattt

SEQ ID NO: 643 - DNA, 70 bp, Homo sapiens
ttctgctccacgggaggtttctgtcctccctgagctcgccttaggacacctgcgttaccgtttgacaggt

SEQ ID NO: 644 - DNA, 70 bp, Homo sapiens
acattctgtgggaataaacacaacttgcttgccctgatgctcaatagcagtgtaatcaccatgtttaaac

SEQ ID NO: 645 - DNA, 70 bp, Homo sapiens
ctttgatgccttgacctatgatttcaatacagcgcattactttggctgctaatttttctgggagggcaca

SEQ ID NO: 646 - DNA, 70 bp, Homo sapiens
gtttctaaaaatagtgttataggctggactgtgtccccttcctgtgccccgccgccatgcatatatggac

SEQ ID NO: 647 - DNA, 70 bp, Homo sapiens
gtcttattattttttgtcgatcaatgcttatcctcgtgttcgttttgatatattaatgtatatgtgttgg

SEQ ID NO: 648 - DNA, 70 bp, Homo sapiens
catctttagtgaaagagtaaatggtggccgagggctccttttgtgagggatgtgccttggtgaagaaggc

SEQ ID NO: 649 - DNA, 70 bp, Homo sapiens
cggatcacttgaggtcaggagttcgagaccagcctccaacatggcaaagccctgtctctactaaaaatac

SEQ ID NO: 650 - DNA, 70 bp, Homo sapiens
catctctccaatctacccaagaggaacccagttacccgaaggcaggttccacagcccactcccagcagca

SEQ ID NO: 651 - DNA, 70 bp, Homo sapiens
ttccctgacccccataccctcacccttaaaattctcctgtaactcaactaacaaaatcaagcctgattca

SEQ ID NO: 652 - DNA, 70 bp, Homo sapiens
ggtcttctaagccaggcaggtgaggcaatttcatgtctgtgatgtgcatccgctccactttatcccttgt

SEQ ID NO: 653 - DNA, 70 bp, Homo sapiens
cacgacggtctaaacccagctcacgttccctattagtgggtgaacaatccaacgcttggtgaattctgct

SEQ ID NO: 654 - DNA, 70 bp, Homo sapiens
gcatcagcaggcagttggttgaagtcagcggaggggtgttccattctttgtttttccagggcttgttttc

SEQ ID NO: 655 - DNA, 70 bp, Homo sapiens
tctatccaactttgccatcttagactagccttctttaccctactgacccatacattggtctctgtatcct

SEQ ID NO: 656 - DNA, 70 bp, Homo sapiens
ctccaccccggtggtgctggtccggaaggacgacctgcacagaaagagactgcacaacacgatagcactg

SEQ ID NO: 657 - DNA, 70 bp, Homo sapiens
ctaaccattcgtgattattaagatagggttgggtcagggcttagggagggggcagaaatattggggatag

SEQ ID NO: 658 - DNA, 70 bp, Homo sapiens
gactacttcccaattaactccaactcacagtgatcctttcaactcatgcggcatctatttttgccaccac

SEQ ID NO: 659 - DNA, 70 bp, Homo sapiens
agccctcagtagacacgtctagggcaggcttgagagatcagatggcgtgaaaggcttgtgatctgttcgt

SEQ ID NO: 660 - DNA, 70 bp, Homo sapiens
gggcctggaatttcctttccacttgatagaagtatatattaggaagtccagttaatagtatttttattta

SEQ ID NO: 661 - DNA, 70 bp, Homo sapiens
cgtccatgccctgagtccaccccggggaaggtgacagcattgcttctgtgtaaattatgtactgcaaaaa

SEQ ID NO: 662 - DNA, 70 bp, Homo sapiens
ttgggatctgaggggtcctctctgtgcccatcacagtttgagcttcagggaaaagaagaagaggtctttg

SEQ ID NO: 663 - DNA, 70 bp, Homo sapiens
cgcaggcaaccaaaactaaagcacccgacgacttagttgctccggtcgtgaagaaaccacacatctatta

SEQ ID NO: 664 - DNA, 70 bp, Homo sapiens
aaacagatagccacaagaggttgggacagaggagggtaaaggctcagaaggaggttcaacctctgactca

SEQ ID NO: 665 - DNA, 70 bp, Homo sapiens
ccacgtggtctcacgttttcatgttgacagccagtcagagtcaagagctcagctgtatcgacagatcgtc

SEQ ID NO: 666 - DNA, 70 bp, Homo sapiens
tgtgaagccaggtgtgggttctactcagtgccatagatagactgagtcttctctcgtaggttaccattac

SEQ ID NO: 667 - DNA, 70 bp, Homo sapiens
gtccaacagaggagggatgtggagagcgtttcaggtgcttttcaggtcagtgcatcagcaaatcattggt

SEQ ID NO: 668 - DNA, 70 bp, Homo sapiens
gaaaacataaccagccattggctatttaaacttgtatttttttatttacaaaatataaatatgaagacat

SEQ ID NO: 669 - DNA, 70 bp, Homo sapiens
agggtgtgggtggctcccctccagggatggctgctccacggtttgcattaaaggttctgtataaggccaa

SEQ ID NO: 670 - DNA, 70 bp, Homo sapiens
agcatggaaacaagatgaaattccatttgtaggtagtgagacaaaattgatgatccattaagtaaacaat

SEQ ID NO: 671 - DNA, 70 bp, Homo sapiens
ccttggttccctaaccctaattgatgagaggctcgctgcttgatggtgtgtacaaactcacctgaatggg

SEQ ID NO: 672 - DNA, 70 bp, Homo sapiens
caatctgaaataaaagtgggatgggagagcgtgtccttcagatcaagggtactaaagtccctttcgctgc

SEQ ID NO: 673 - DNA, 70 bp, Homo sapiens
tgatggcgccttcaaagaggtgaagctgtcggactacaaagggaagtacgtggtcctctttttctaccct

SEQ ID NO: 674 - DNA, 70 bp, Homo sapiens
ggagtagctgagatcttagaagccgtcacctacactcaagcctcgcccaaagaagcaaaagttgaaccca

SEQ ID NO: 675 - DNA, 70 bp, Homo sapiens
agggaatagaaatgaaacaaattatctctcatcttttgactatttcaagtctaataaattcttaattaac

SEQ ID NO: 676 - DNA, 70 bp, Homo sapiens
cccagaaaacagaagtttctactgtctcgtctacccaagttggccccaactgaggacccaatattggcct

SEQ ID NO: 677 - DNA, 70 bp, Homo sapiens
cgtgtgattggtgcaggagaattcggtgaagtctgcagtggccgtttgaaacttccagggaaaagagatg

SEQ ID NO: 678 - DNA, 70 bp, Homo sapiens
caaaactggatggcatccgaattgtctggaagttttgtcttgggcatgatgggctgggccaaatgaaatg

SEQ ID NO: 679 - DNA, 70 bp, Homo sapiens
cacccttcaggggatgagaagttttcaaggggtattactcaggcactaaccccaggttagatgacagcac

SEQ ID NO: 680 - DNA, 70 bp, Homo sapiens
tggaggaccgaaccgtagtacgctaaaaagtgcccggatggacttgtggatagtggtgaaattccaatcg

SEQ ID NO: 681 - DNA, 70 bp, Homo sapiens
taagcttgcgttgattaagtccctgccctttgtacacaccgcccgtcgctactaccgattggatggttta

SEQ ID NO: 682 - DNA, 70 bp, Homo sapiens
catcattcagatggctttccagatgaccaggacgagtgggatattttgcccccaacttggctcggcatgt

SEQ ID NO: 683 - DNA, 70 bp, Homo sapiens
ctgactattactgtaactcccgggacagcagtggtaaccgtctggtattcggcggagggaccaagctgac

SEQ ID NO: 684 - DNA, 70 bp, Homo sapiens
gcgcgctcgccccgccgctcctgctgcagccccaggcccctcgccgccgccaccatggacgccatcaaga

SEQ ID NO: 685 - DNA, 70 bp, Homo sapiens
agcgagttctacatcctaacggcagcccactgtctctaccaagccaagagattcgaaggggaccggaaca

SEQ ID NO: 686 - DNA, 70 bp, Homo sapiens
gatctcggatgaccaaaccagccttcggagcgttctctgtcctacttctgactttacttgtggtgtgaca

SEQ ID NO: 687 - DNA, 70 bp, Homo sapiens
tggtcctgcgcttgagggggggtgtctaagtttccccttttaaggtttcaacaaatttcattgcactttc

SEQ ID NO: 688 - DNA, 70 bp, Homo sapiens
aagacgaatagtcaaaagggagcctcttctacctggatgaaggcaattgtgtcatcggggacactaggtg

SEQ ID NO: 689 - DNA, 70 bp, Homo sapiens
cctacattccctctcctgcccagatgccctttggaaagccattgaccacccaccatattgtttgatctac

SEQ ID NO: 690 - DNA, 70 bp, Homo sapiens
aacatgacaaggaattcttccacccacgctaccaccatcgagagttccggtttgatctttccaagatccc

SEQ ID NO: 691 - DNA, 70 bp, Homo sapiens
accgtgacaattggcctccgggggccactttcggcggagggaccaaggtggagatcaaacataccaccgg

SEQ ID NO: 692 - DNA, 70 bp, Homo sapiens
ccaactaccgcgcttatgccacggagccgcacgccaagaaaaaatctaagatctccgcctcgagaaaatt

SEQ ID NO: 693 - DNA, 70 bp, Homo sapiens
agattccaggcggtgcaaccctggtgttcgaggtggagctgctcaaaatagagcgacgaactgagctgta

SEQ ID NO: 694 - DNA, 70 bp, Homo sapiens
gcttcgagggtgtgaagggaaagaagaagatgtcagcagcagaggcagtgaaagaaaaatggctcccgta

SEQ ID NO: 695 - DNA, 70 bp, Homo sapiens
ataggaagtagacctctttttcttaccagtctcctcccctactctgccccctaagctggctgtacctgtt

SEQ ID NO: 696 - DNA, 70 bp, Homo sapiens
agcgccgctgtgactcggggtgacctccgcatcctgcctgaggcccatcagcgcacatggcatgcctgga

SEQ ID NO: 697 - DNA, 70 bp, Homo sapiens
gcaagtggtcaacagcaggtggctgtcgagacgtctaatgaccattctccatatacctttcaacctaata

SEQ ID NO: 698 - DNA, 70 bp, Homo sapiens
ccgctaggggtgcggggttggggaggaggccgctagtctacgcctgtggagccgatactcagccctctgc

SEQ ID NO: 699 - DNA, 70 bp, Homo sapiens
gcgcagatagcacttcagctcggccatctcagatcccaactccagtgaataacaacacaaagaagcgaga

SEQ ID NO: 700 - DNA, 70 bp, Homo sapiens
tagctggacaggccctgcccctcaccagcaagaggcatgattggatggagcttctaatgtcattcaaaaa

SEQ ID NO: 701 - DNA, 70 bp, Homo sapiens
gtatatctttaattctgggagaaatgagataaaagatgtacttgtgaccattgtaacaatagcacaaata

SEQ ID NO: 702 - DNA, 70 bp, Homo sapiens
tcaagactttaccactggttgactcaaaagattcaatgatcctgctgggctcggtggagcggtcggaact

SEQ ID NO: 703 - DNA, 70 bp, Homo sapiens
atggggacttgtgaatttttctaaaggtgctatttaacatgggaggagagcgtgtgcgctccagcccagc

SEQ ID NO: 704 - DNA, 70 bp, Homo sapiens
catgtctcccatcagaaagattcattggcatgccacagggattctcctccttcatcctgtaaaggtcaac

SEQ ID NO: 705 - DNA, 70 bp, Homo sapiens
agccgccgcgtcccctcgccgagtcccctcgccagattccctccgtcgccgccaagatgatgtgcggggc

SEQ ID NO: 706 - DNA, 70 bp, Homo sapiens
cataaatcaactgtccatcaggtgaggtgtgctccatacccagcggttcttcatgagtagtgggctatgc

SEQ ID NO: 707 - DNA, 70 bp, Homo sapiens
tatctatattttacataaatttagtattttgtttcagtgcactaatatgtaagacaaaaaggactactta

SEQ ID NO: 708 - DNA, 70 bp, Homo sapiens
ctcgcgtctcactcagtgtaccttctagtcccgccatggccgctctcacccgggacccccagttccagaa

SEQ ID NO: 709 - DNA, 70 bp, Homo sapiens
cgaaatatcaatgcaaactaggatatgtaacagcagatggtgaaacatcaggatcaattagatgtgggaa

SEQ ID NO: 710 - DNA, 70 bp, Homo sapiens
tttactaagtaaaagggtggagaggttcctggggtggattcctaagcagtgcttgtaaaccatcgcgtgc

SEQ ID NO: 711 - DNA, 70 bp, Homo sapiens
atagatcttggccctgttaaggcatccacttcacagttctgaaggctgagtcagccccactccacagtta

SEQ ID NO: 712 - DNA, 70 bp, Homo sapiens
gcggatcagtgatagccatgaggacactgggattctggacttcagctcactgctgaaaaagagagacagt

SEQ ID NO: 713 - DNA, 70 bp, Homo sapiens
aaaaaagactttgagctgaatgctctcaacgcaaggattgaggatgaacaggccctcggcagccagctgc

SEQ ID NO: 714 - DNA, 70 bp, Homo sapiens
catcaactatgaagcatttgtgaagcacatcatgtccagctaaacctcgtgcccagaagccaggaaggct

SEQ ID NO: 715 - DNA, 70 bp, Homo sapiens
gaaattcttggaaacttccattaagtgtgtagattgagcaggtagtaattgcatgcagtttgtacattag

SEQ ID NO: 716 - DNA, 70 bp, Homo sapiens
agcccttgcaaaaacacggcttgtggcattggcatacttgcccttacaggtggagtatcttcgtcacaca

SEQ ID NO: 717 - DNA, 70 bp, Homo sapiens
tttacttggtataatatacatggttaaaatgcttatgtgacttcgagtaggtgaatcttaaagaaataaa

SEQ ID NO: 718 - DNA, 70 bp, Homo sapiens
ctgctatagcggtgtcatgttggatcgctttgtgactgttcatctgtccttgacagtggctgtcatcttg

SEQ ID NO: 719 - DNA, 70 bp, Homo sapiens
acaaggagtcagacattttaagatggtggcagtagaggctatggacagggcatgccacgtgggctcatat

SEQ ID NO: 720 - DNA, 70 bp, Homo sapiens
caaagccagacaagccaacgacacagctaaagatgtactggcacagattacagagctccaccagaacctc

SEQ ID NO: 721 - DNA, 70 bp, Homo sapiens
aggcgcggaggtctggcctataaagtagtcgcggagacggggtgctggtttgcgtcgtagtctcctgcag

SEQ ID NO: 722 - DNA, 70 bp, Homo sapiens
ttggaggcattcctacttacggggttggagctgggggctttcccggctttggtgtcggagtcggaggtat

SEQ ID NO: 723 - DNA, 70 bp, Homo sapiens
ccgtctgtcctttgtccacaaggaatttccctgggcgctaattatgagggaggcgtgtagcttcttatca

SEQ ID NO: 724 - DNA, 70 bp, Homo sapiens
ctggtattatctctctatcagataagattttgttaatgtactattttactcttcaataaataaaacagtt

SEQ ID NO: 725 - DNA, 70 bp, Homo sapiens
agatgggtgctggtcctgttgatcccagtctctgccagaccaaggcgagtttccccactaataaagtgcc

SEQ ID NO: 726 - DNA, 70 bp, Homo sapiens
gtgctacacccttttccagctggatgagaatttgagtgctctgatccctctacagagcttccctgactca

SEQ ID NO: 727 - DNA, 70 bp, Homo sapiens
atcccagtggaggggacccttttacttgccctgaacatacacatgctgggccattgtgattgaagtcttc

SEQ ID NO: 728 - DNA, 70 bp, Homo sapiens
aaaagccacggaccgttgcacaaaaaggaaagtttgggaagggatgggagagtggcttgctgatgttcct

SEQ ID NO: 729 - DNA, 70 bp, Homo sapiens
atgccggcctccctgttgtccactgccccagccacatcatccctgtgcgggttgcagatgctgctaaaaa

SEQ ID NO: 730 - DNA, 70 bp, Homo sapiens
ccccaaaccataaaaccctatacaagttgttctagtaacaatacatgagaaagatgtctatgtagctgaa

SEQ ID NO: 731 - DNA, 70 bp, Homo sapiens
tgcactccagccggggtgacagaagagaccttgtctcgaaaacgaatctgaaaacaatggaaccatgcct

SEQ ID NO: 732 - DNA, 70 bp, Homo sapiens
gaggacctccgctgcaaatacatctccctcatctacaccaactatgaggcgggcaaggatgactatgtga

SEQ ID NO: 733 - DNA, 70 bp, Homo sapiens
gaggccttgtgtcctttaatcactgcatttcattttgattttggataataaacctggctcagcctgagcc

SEQ ID NO: 734 - DNA, 70 bp, Homo sapiens
tccaaggcaggtcatcctgacactgcaacccactttggtggctgtgggcaagtccttcaccattgagtgc

SEQ ID NO: 735 - DNA, 70 bp, Homo sapiens
taatgctctgggaggatggggagaactacagaattcggtaaagacatttggggagacacatcctttcacc

SEQ ID NO: 736 - DNA, 70 bp, Homo sapiens
ttccccaattatcctccttcactccctgtcatagttaccgatggtgtcccgttgtgtgggtttactctgt

SEQ ID NO: 737 - DNA, 70 bp, Homo sapiens
cgtaagggctacagtcgaaaagggtttgaccggcttagcactgagggcagtgaccaagagaaagaggatg

SEQ ID NO: 738 - DNA, 70 bp, Homo sapiens; identical to SEQ ID NO: 241

SEQ ID NO: 739 - DNA, 70 bp, Homo sapiens
tgagagctaaacccagcaattttctatgattttttcagatatagataataaacttatgaacagcaactaa

SEQ ID NO: 740 - DNA, 70 bp, Homo sapiens
ggctggaaccatggagggtgtagaagagaagaagaaggaggttcctgctgtgccagaaacccttaagaaa

SEQ ID NO: 741 - DNA, 70 bp, Homo sapiens
aagaacttgccactaaactgggttaaatgtacactgttgagttttctgtacataaaaataattgaaataa

SEQ ID NO: 742 - DNA, 70 bp, Homo sapiens
ggtgctgtggaatgcccagccagttaagcacaaaggaaaacatttcaataaaggatcatttgacaactgg

SEQ ID NO: 743 - DNA, 70 bp, Homo sapiens
cgggccagccgaggctacaaaaactaaccctggatcctactctcttattaaaaagatttttgctgacaaa

SEQ ID NO: 744 - DNA, 70 bp, Homo sapiens
taatcatgtcgtcgccaagtcccgcttctggtactttgtatctcagttaaagaagatgaagaagtcttca

SEQ ID NO: 745 - DNA, 70 bp, Homo sapiens
gaggagatcatcaagactttatccaaggaggaagagaccaagaaataaaacctcccactttgtctgtaca

SEQ ID NO: 746 - DNA, 70 bp, Homo sapiens
ttctcgtggtaataccagagtagaaggagagggtgactttaccgaactgacagccattggggaggcagat

SEQ ID NO: 747 - DNA, 70 bp, Homo sapiens
tcttgctgatataatggccaagaggaatcagaaacctgaagttagaaaggctcaacgagaacaagctatc

SEQ ID NO: 748 - DNA, 70 bp, Homo sapiens
tgaggaaatctgaaatagagtactatgctatgttggctaaaactggtgtccatcactacagtggcaataa

SEQ ID NO: 749 - DNA, 70 bp, Homo sapiens
cccaggctgtttggcgctgcccaggaatggtatcaattcccctgtttctcttgtagccagttactagaat

SEQ ID NO: 750 - DNA, 70 bp, Homo sapiens
ctgtccaatagaaaaagttggtgtgctggagctacctcacctcagcttgagagagccagttgtgtgcatc

SEQ ID NO: 751 - DNA, 70 bp, Homo sapiens
gccaaggaagagtcggaggagtcggacgaggatatgggatttggtctctttgactaatcaccaaaaagca

SEQ ID NO: 752 - DNA, 70 bp, Homo sapiens
gagcgcggcggcaagatggcagtgcaaatatccaagaagaggaagtttgtcgctgatggcatcttcaaag

SEQ ID NO: 753 - DNA, 70 bp, Homo sapiens
tcggacgccggattttgacgtgctctcgcgagatttgggtctcttcctaagccggcgctcggcaagttct

SEQ ID NO: 754 - DNA, 70 bp, Homo sapiens
gccaagctgactcctgaggaagaagagattttaaacaaaaaacgatctaaaaaaattcagaagaaatatg

SEQ ID NO: 755 - DNA, 70 bp, Homo sapiens
ttcctctccagcccctgcgtaatcgataaggaaacccggacgctgctgcccctttctttttttcaggcgg

SEQ ID NO: 756 - DNA, 70 bp, Homo sapiens
tttcgttgcctgatcgccgccatcatgggtcgcatgcatgctcccgggaagggcctgtcccagtcggctt

SEQ ID NO: 757 - DNA, 70 bp, Homo sapiens
tcttttaccaaggacccgccaacatgggccgcgttcgcaccaaaaccgtgaagaaggcggcccgggtcat

SEQ ID NO: 758 - DNA, 70 bp, Homo sapiens
aacgacgcaaacgaagccaagttcccccagctccgaacaggagctctctatcctctctctattacactcc

SEQ ID NO: 759 - DNA, 70 bp, Homo sapiens
gttgaggtggaagtcaccattgcagatgcttaagtcaactattttaataaattgatgaccagttgttaaa

SEQ ID NO: 760 - DNA, 70 bp, Homo sapiens
gctggtgaagatgcatgaataggtccaaccagctgtacatttggaaaaataaaactttattaaatcaaaa

SEQ ID NO: 761 - DNA, 70 bp, Homo sapiens
gcctcgtcgaaggtgctaaaaagatcaaagttgcagaactgttagccaacatgccagaccccactcagga

SEQ ID NO: 762 - DNA, 70 bp, Homo sapiens
ctattccctcaaatctgagggagctgagtaacaccatcgatcatgatgtagagtgtggttatgaacttta

SEQ ID NO: 763 - DNA, 70 bp, Homo sapiens
tatttgtatgtggggagtaggtgtttgaggttcccgttctttcccttcccaagtctctgggggtggaaag

SEQ ID NO: 764 - DNA, 70 bp, Homo sapiens
cggagaagaatcggatcaataaggccgtatctgaggaacagcagcctgcactcaagggcaaaaagggaaa

SEQ ID NO: 765 - DNA, 70 bp, Homo sapiens
agtgggtggaggcagccagggcttacctgtacactgacttgagaccagttgaataaaagtgcacacctta

SEQ ID NO: 766 - DNA, 70 bp, Homo sapiens
gaccggttaaggagaagccagagttagagtaggagaggactaattctcagcagcagtggaggtgagttct

SEQ ID NO: 767 - DNA, 70 bp, Homo sapiens
tattgatgggcccaagcgtaaccaggctcttctgattggccggtgtacttcagtttccgtccaaggtccg

SEQ ID NO: 768 - DNA, 70 bp, Homo sapiens
tcttttgtggttgttgctggcccaatgagtccctagtcacatcccctgccagagggagttcttcttttgt

SEQ ID NO: 769 - DNA, 70 bp, Homo sapiens
gagggcagggaccgtatcttatttactgttagtatccgttgcatctagtgtggtgcacctggcacacagt

SEQ ID NO: 770 - DNA, 70 bp, Homo sapiens
tggcaagagagcctcacacctcactaggtgcagagagcccaggccttatgttaaaatcatgcacttgaaa

SEQ ID NO: 771 - DNA, 70 bp, Homo sapiens
ggagcctctttgtagggactgtgcctaggtagcatgtcctaacatttgttctggtcttgcataacttcag

SEQ ID NO: 772 - DNA, 70 bp, Homo sapiens
gtatcccgcgggtggaggccggggtggcgccggccggggcgggggagcccaaaagaccggctgccgcctg

SEQ ID NO: 773 - DNA, 70 bp, Homo sapiens
gtagggatggggctgtggggatagtgaggcatcgcaatgtaagactcgggattagtacacacttgttgat

SEQ ID NO: 774 - DNA, 70 bp, Homo sapiens; identical to SEQ ID NO: 460

SEQ ID NO: 775 - DNA, 70 bp, Homo sapiens
gcctgcaccagtgccgtcctgctgatgtggtaggctagcaatattttggttaaaatcatgtttgtggccg

SEQ ID NO: 776 - DNA, 70 bp, Homo sapiens
tcactccttaaattcacactttgccacttaactccagtgtggatgacagagcgagaccctgcctcaaaaa

SEQ ID NO: 777 - DNA, 70 bp, Homo sapiens
gccctgggcagccagcattcattgtaagttccctctttgaaaactggtgtgtgggtgttcagttctgtgt

SEQ ID NO: 778 - DNA, 70 bp, Homo sapiens
agaaaaaagtcacgttaaatggtttcttggacacgcttatgtcagatcctcccccgcagtgtctggtctg

SEQ ID NO: 779 - DNA, 70 bp, Homo sapiens
catgtgggcaaagccttcaatcagggcaagatcttcaagtgaacatctcttgccatcacctagctgcctg

SEQ ID NO: 780 - DNA, 70 bp, Homo sapiens
atgaagccaggattcagtccccgtgggggtggctttggcggccgagggggctttggtgaccgtggtggtc

SEQ ID NO: 781 - DNA, 70 bp, Homo sapiens
gcagctatttcaaagtgtgttggattaattaggatcatccctttggttaataaataaatgtgtttgtgct

SEQ ID NO: 782 - DNA, 70 bp, Homo sapiens
gaccagttgttatttacagctctgtaacctcccgttgcgtcaagtctaaaccaagattatgtgacttgca

SEQ ID NO: 783 - DNA, 70 bp, Homo sapiens
cacttcacagtaaatgccaaagctgctggcaaaggcaagctggacgtccagttctcaggactcaccaagg

SEQ ID NO: 784 - DNA, 70 bp, Homo sapiens
aggaagttatgggaatacctgtggtggttgtgatccctaggtcttgggagctcttggaggtgtctgtatc

SEQ ID NO: 785 - DNA, 70 bp, Homo sapiens
ctcactgggtggctttgcctatgtggagatcagctccaaagagatgactgtcacttacatcgaggcctcg

SEQ ID NO: 786 - DNA, 70 bp, Homo sapiens
agcgtgagattgtccgggacatcaaggagaaactgtgttatgtagctctggactttgaaaatgagatggc

SEQ ID NO: 787 - DNA, 70 bp, Homo sapiens
tagaatcctcaaccgtgcggaccatcaaccttcgagaaattccagttgtctttttcccagccgcatcctg

SEQ ID NO: 788 - DNA, 70 bp, Homo sapiens
caaccacgacaaaggaagttgacctaaacatgtaaccatgccctaccctgttaccttgctagctgcaaaa

SEQ ID NO: 789 - DNA, 70 bp, Homo sapiens
gaggctctgtaaccttatctaagaacttggaagccgtcagccaagtcgccacatttctctgcaaaatgtc

SEQ ID NO: 790 - DNA, 70 bp, Homo sapiens
taggcggagcctcggccgcgggccgccttggtatatctgcgtgcgcgcgtctgctgggccagtcgggaca

SEQ ID NO: 791 - DNA, 70 bp, Homo sapiens
ctagcggttacgccaacgcgcgcgtgcgcccttgcgcgtttctctcttcccactcgggtttgacctacag

SEQ ID NO: 792 - DNA, 70 bp, Homo sapiens
cgcaaggaggggctgcttctgaggtcggtggctgtctttccattaaagaaacaccgtgcaacgtgaaaaa

SEQ ID NO: 793 - DNA, 70 bp, Homo sapiens
ggagttggtcaaatgagggaacatctgggttatgccttttttaaagtagttttctttaggaactgtcagc

SEQ ID NO: 794 - DNA, 70 bp, Homo sapiens
aggcatctggagagtccaggagaggagactcacctctgtcgcttgggttaaacaagagacaggttttgta

SEQ ID NO: 795 - DNA, 70 bp, Homo sapiens
aactaatccatcaccggggtggtttagtggctcaacattgtgttcccatttcagctgatcagtgggcctc

SEQ ID NO: 796 - DNA, 70 bp, Homo sapiens
acaatttgtttcagagaagagagttgaacagtggtgagctgggctcacagctccatccatgggccccatt

SEQ ID NO: 797 - DNA, 70 bp, Homo sapiens
tgttgacacaggtctttcctaaggctgcaaggtttaggctggtggcccaggaccatcatcctactgtaat

SEQ ID NO: 798 - DNA, 70 bp, Homo sapiens
ctgggggagtggaatagtatcctccaggtttttcaattaaacggattattttttcagaccgaaaagaaaa

SEQ ID NO: 799 - DNA, 70 bp, Homo sapiens; identical to SEQ ID NO: 261

SEQ ID NO: 800 - DNA, 70 bp, Homo sapiens
ggccacttttcactaacagaagtcacaagccaagtgagacactcatccaagaggaaggatggccagtatc

SEQ ID NO: 801 - DNA, 70 bp, Homo sapiens
agaccagagatagtggggagacttcttggcttggtgaggaaaagcggacatcagctggtcaaacaaactc

SEQ ID NO: 802 - DNA, 70 bp, Homo sapiens
ccatggatgagaaagtcgagttgtatcgcattaagttcaaggagagctttgctgagatgaacaggggctc

SEQ ID NO: 803 - DNA, 70 bp, Homo sapiens
gtttttcagctcacttcaagggtacctgaagcgaattggcaccaaagcagcagctgtattgccgcagttc

SEQ ID NO: 804 - DNA, 70 bp, Homo sapiens
ggatagataattttatttgaaattttacacactgaaagctctaaataaacagatacattcacattcaaaa

SEQ ID NO: 805 - DNA, 70 bp, Homo sapiens
agtttccccaccagtgaatgaaagtcttgtgactagtgctgaagcttattaatgctaagggcaggcccaa

SEQ ID NO: 806 - DNA, 70 bp, Homo sapiens
ggggaggcatcagtgtccttggcaggctgatttctaggtaggaaatgtggtagctcacgctcacttttaa

SEQ ID NO: 807 - DNA, 70 bp, Homo sapiens
gacgcggctcaaaaggaaaccaagtggtcaggagttgtttctgacccactgatctctactaccacaagga

SEQ ID NO: 808 - DNA, 70 bp, Homo sapiens
cggccgaacccagacccgaggttttagaagcagagtcaggcgaagctgggccagaaccgcgacctccgca

SEQ ID NO: 809 - DNA, 70 bp, Homo sapiens
cttgaaattgtccccgtggtctcttactttcctttccccagcccagggtggacttagaaagcaggggcta

SEQ ID NO: 810 - DNA, 70 bp, Homo sapiens
caggggccaggggaacccgtgaggatcactctcaaatgagattaaaaacaaggaagcagagaatggtcag

SEQ ID NO: 811 - DNA, 70 bp, Homo sapiens
ccaagtccctgaagtctggagacgcggccatcgtggagatggtgccgggaaagcccatgtgtgtggagag

SEQ ID NO: 812 - DNA, 70 bp, Homo sapiens
acaaggtggggacagacttgctggaggaggagatcaccaagtttgaggagcacgtgcagagtgtcgatat

SEQ ID NO: 813 - DNA, 70 bp, Homo sapiens
aacgcattaagaggtttatttgggtacatggcccgcagtggcttttgccccagaaaggggaaaggaacac

SEQ ID NO: 814 - DNA, 70 bp, Homo sapiens
cctgccctgcacccttgtacagtgtctgtgccatggatttcgtttttcttggggtactcttgatgtgaag

SEQ ID NO: 815 - DNA, 70 bp, Homo sapiens
gaagaagggccccaatgccaactcttaagtcttttgtaattctggctttctctaataaaaaagccactta

SEQ ID NO: 816 - DNA, 70 bp, Homo sapiens
aaagtgtgaatgtgggtgtcggctgcggcattaaattcatcatctcaacccagagtgtctggtctccctg

SEQ ID NO: 817 - DNA, 70 bp, Homo sapiens
cttttccctatccacaggggtgtttgtgtgtgtgcgcgtgtgcgtttcaataaagtttgtacactttcaa

SEQ ID NO: 818 - DNA, 70 bp, Homo sapiens
atgcgcagcagcggcgccgacgcggggcggtgcctggtgaccgcgcgcgctcccggaagtgtgccggcgt

SEQ ID NO: 819 - DNA, 70 bp, Homo sapiens
ggggtcaaaaggtacctaagtatatgattgcgagtggaaaaataggggacagaaatcaggtattggcagt

SEQ ID NO: 820 - DNA, 70 bp, Homo sapiens
atcagttcttaatttaatttttaagtattgttttactcctttttattcatacgtaaaattttggattaat

SEQ ID NO: 821 - DNA, 70 bp, Homo sapiens
aaagagggtccatcaaagagatgagccatcaccccccaggacacacagtggtcaaggatagaagccattt

SEQ ID NO: 822 - DNA, 70 bp, Homo sapiens
gcacggcatggattaacacggcagaggaacaaaggtgtgctctgagcttcttcatatttcaccttcaccc

SEQ ID NO: 823 - DNA, 70 bp, Homo sapiens
ggctatgcaacagctctcacctacgcgagtcttactttgagttagtgccataacagaccactgtatgttt

SEQ ID NO: 824 - DNA, 70 bp, Homo sapiens
gtacagtcgccgcgtgcggagcttgttactggttacttggcctcatggcggtccgagcttcgttcgagaa

SEQ ID NO: 825 - DNA, 70 bp, Homo sapiens
agcatattgtctggggattgttgggacaggttttggtgactctgtgcccttgctctctaacttctgagcc

SEQ ID NO: 826 - DNA, 70 bp, Homo sapiens
agacacatggaacaaagaagctgtgaccccagcaggatgtctaatatgtgaggaaatgagatgtccacct

SEQ ID NO: 827 - DNA, 70 bp, Homo sapiens
agttcgttgtgctgtttctgactcctaatgagagttccttccagaccgttagctgtctccttgccaagcc

SEQ ID NO: 828 - DNA, 70 bp, Homo sapiens
gctccaggttgggtgctcacagaacccttttcctgactctcatggaagatggtggaaggaaaatagactg

SEQ ID NO: 829 - DNA, 70 bp, Homo sapiens
aaaaaatcttacacatctgccaccggaaataccatgcacagagtccttaaaaaatagagtgcagtattta

SEQ ID NO: 830 - DNA, 70 bp, Homo sapiens
gttgaaggggctggtgccactgggacccgaatcaagtcgacacactacgttgagtttattaacaaaagcc

SEQ ID NO: 831 - DNA, 70 bp, Homo sapiens
agaagacaaagagcaaggggccctacatctgcgctctgtgcgccaaggagttcaagaacggctacaatct

SEQ ID NO: 832 - DNA, 70 bp, Homo sapiens
cctaccccgaactccaaaaattacacctggagtcaggtgcagaagggaaccttgtatttcacaggcctca

SEQ ID NO: 833 - DNA, 70 bp, Homo sapiens
accacagtggtgtccgagaagtcaggcacgtagctcagcggcggccgcggcgcgtgcgtctgtgcctctg

SEQ ID NO: 834 - DNA, 70 bp, Homo sapiens
acagtaagattgaggatgagcaggcgctggcccttcaactacagaagaaactgaaggaaaaccaggcacg

SEQ ID NO: 835 - DNA, 70 bp, Homo sapiens
tgaggctcccaaggaacctgcctttgaccccaagagtgtaaagatagacttcactgccgaccagattgaa

SEQ ID NO: 836 - DNA, 70 bp, Homo sapiens
ccaaaatacttgcatccaaggttctagtctctgttgctgtgctggtctttagccccactgctggcactga

SEQ ID NO: 837 - DNA, 70 bp, Homo sapiens
gagtgtgtctcatgctttcagatgtgcatatgagcagaattaattaaacatttgcctatgactccaacaa

SEQ ID NO: 838 - DNA, 70 bp, Homo sapiens
atattgcaaaaggatgtgtgtctttctccccgagctcccctgttccccttcattgaaaaccaccacggtg

SEQ ID NO: 839 - DNA, 70 bp, Homo sapiens; identical to SEQ ID NO: 623

SEQ ID NO: 840 - DNA, 70 bp, Homo sapiens
taatcattttctagaaagtatgggtatctatactaatgtttttatatgaagaacataggtgtctttgtgg

SEQ ID NO: 841 - DNA, 70 bp, Homo sapiens
aatgtaactatttagccctggattatacatactgtccaattttcattaaatttttgtcttataactataa

SEQ ID NO: 842 - DNA, 70 bp, Homo sapiens
ttggctgccggtgagttgggtgccggtggagtcgtgttggtcctcagaatccccgcgtagccgctgcctc

SEQ ID NO: 843 - DNA, 70 bp, Homo sapiens
ttactactgtgggtttaaagccactgcagcgggagttaaacaaactgagtcaaccagcttccttgaaaaa

SEQ ID NO: 844 - DNA, 70 bp, Homo sapiens
gcagccatctcgccgtgagacagcaagtgtcgcgcagccgtgcgatgttgtcctctacagccatgtattc

SEQ ID NO: 845 - DNA, 70 bp, Homo sapiens
ggaagtgagtggacagcctttgtgtgtatctctccaataaagctctgtgggccaagtcctctaggaaaaa

SEQ ID NO: 846 - DNA, 70 bp, Homo sapiens
aaatctgggttcaaccagcccctgccatttcttaagactttctgctgcactcacaggatcctgagctgca

SEQ ID NO: 847 - DNA, 70 bp, Homo sapiens
taaggtagcaggcagtccagccctgatgtggagacacatgggattttggaaatcagcttctggaggaatg

SEQ ID NO: 848 - DNA, 70 bp, Homo sapiens; identical to SEQ ID NO: 303

SEQ ID NO: 849 - DNA, 70 bp, Homo sapiens
catcttgggttacccactctgtccactcccataggctacagaaaaagtcacaagcgcatggtttccaacc

SEQ ID NO: 850 - DNA, 70 bp, Homo sapiens
tttttccaccctggctccttcagacacgtgcttgatgctgagcaagttcaataaagattcttggaagttt

SEQ ID NO: 851 - DNA, 70 bp, Homo sapiens
tgccatgtactattttacctatgacccgtggattggcaagttattgtatcttgaggacttcttcgtgatg

SEQ ID NO: 852 - DNA, 70 bp, Homo sapiens
gccctgccaccgtggggagtctggtttttctcttcatcctgtctctctcctccttactcttggataaata

SEQ ID NO: 853 - DNA, 70 bp, Homo sapiens
aggccgagctctgcagagcttacaattgagactgctaacccctacctttgaagggatcaacggattgttg

SEQ ID NO: 854 - DNA, 70 bp, Homo sapiens
ccatctctaggatgtcgtctttggtgagatctctattatatcttgtatggtttgcaaaagggcttcctaa

SEQ ID NO: 855 - DNA, 70 bp, Homo sapiens
tgttggtttattgctggcaacgtgaattctctcaggggtctaggaggggcattttggagactgcctgaca

SEQ ID NO: 856 - DNA, 70 bp, Homo sapiens; identical to SEQ ID NO: 414

SEQ ID NO: 857 - DNA, 70 bp, Homo sapiens
atggttccaggactacaatgtctttatttttaactgtttgccactgctgccctcacccctgcccggctct

SEQ ID NO: 858 - DNA, 70 bp, Homo sapiens
gaccatcacatcccttcaagagtcctgaagatcaagccagttctccttccctgcagagctttggccatta

SEQ ID NO: 859 - DNA, 70 bp, Homo sapiens
aggagggtcttcgaggggcctgggggcgggggactaagatggacgcctgggaagggaactgggaggcagc

SEQ ID NO: 860 - DNA, 70 bp, Homo sapiens
tgtcctcaaccccaaatcccccgactccctccccagatctgtcctgggggatgcaaataaagcctgctct

SEQ ID NO: 861 - DNA, 70 bp, Homo sapiens
gccgtgcttctgcccctacaaggtttgggccgaggtgggggagggtcctggttgccggccccgcccggtc

SEQ ID NO: 862 - DNA, 70 bp, Homo sapiens; identical to SEQ ID NO: 62

SEQ ID NO: 863 - DNA, 70 bp, Homo sapiens
gtgcttgtggacatcaggcctcctgccagcagttcttgaagcttctttttcattcctgctactctacctg

SEQ ID NO: 864 - DNA, 70 bp, Homo sapiens
ggcgggaggatcacttgaggccaggactttgagaccagccagggcaacataataagacttttctctactt

SEQ ID NO: 865 - DNA, 70 bp, Homo sapiens
cccaagtgcactcatccaggtcagtgctcagatgtgtttaaggagaccctatattcagggaagttgcgtg

SEQ ID NO: 866 - DNA, 70 bp, Homo sapiens
cctaggttcagagcatgggtgctctgagggacaaagttggattagtataagggagctggagcagctgata

SEQ ID NO: 867 - DNA, 70 bp, Homo sapiens
ggcaggacctgtggccaagttcttagttgctgtatgtctcgtggtaggactgtagaaaagggaactgaac

SEQ ID NO: 868 - DNA, 70 bp, Homo sapiens
ggtgcctgatacctctcagcatttgagggccttttctcttcctgcttcatctctaaaggtccttctagga

SEQ ID NO: 869 - DNA, 70 bp, Homo sapiens
tcaccacgtctggtcgaaagatggcagagctgccggtggaccccatgctgtccaaaatgatcttagcctc

SEQ ID NO: 870 - DNA, 70 bp, Homo sapiens
aaaggataaaccccgatattgggacctcacagtgggtgtctgaaaggacagatcactccggagtatcagg

SEQ ID NO: 871 - DNA, 70 bp, Homo sapiens
aagagaaatacacacttctgagaaactgaaacgacaggggaaaggaggtctcactgagcaccgtcccagc

SEQ ID NO: 872 - DNA, 70 bp, Homo sapiens
aatgaggagtgatcatggctacctcagagctgagctgcgaggtgtcggaggagaactgtgagcgccggga

SEQ ID NO: 873 - DNA, 70 bp, Homo sapiens
gaattctcagctcttgggaacccccttgctcccaggggaggggaaacctttttcattcaacattgtaggg

SEQ ID NO: 874 - DNA, 70 bp, Homo sapiens
aaattcctaaaactgtggaatggatcacgtagacatgtaacccagcagcagtttgcttctgttgtccact

SEQ ID NO: 875 - DNA, 70 bp, Homo sapiens
gtaccattcagaatggactgtttgtacgaagcatgtataatgcagttatcttctttctttcgtcgcagcc

SEQ ID NO: 876 - DNA, 70 bp, Homo sapiens
cttctcctcgaccagccatcatgacatttaccatgaatttacttcctcccaagagtttggactgcccgtc

SEQ ID NO: 877 - DNA, 70 bp, Homo sapiens
cctggcttcattctgctctctcttggcacccgacccttggcagcatgtaccacacagccaagctgagact

SEQ ID NO: 878 - DNA, 70 bp, Homo sapiens
agttatcattaccatgttggtgacctgttcagtttgctgctatctcttttggctgattgcaattctggcc

SEQ ID NO: 879 - DNA, 70 bp, Homo sapiens
ccgcgagatctagcatctctgaaatcctggctgtcgaggctttgaagcatgtgttacctggttaagcttg

SEQ ID NO: 880 - DNA, 70 bp, Homo sapiens
acgaggaaaatggcgctagctcggaagctaccgaggtgctaggagttgccgaagcaagtccggaagctac

SEQ ID NO: 881 - DNA, 70 bp, Homo sapiens
ttgaaaattaaacgtgcttggggttcagctggtgaggctgtccctgtaggaagaaagctctgggactgag

SEQ ID NO: 882 - DNA, 70 bp, Homo sapiens
tgaagctggtggtgtctcggggcggcctgttgggagatcttgcatccagcgacgtggccgtggaactgcc

SEQ ID NO: 883 - DNA, 70 bp, Homo sapiens
tccatgtttgatgtatctgagcaggttgctccacaggtagctctaggagggctggcaacttagaggtggg

SEQ ID NO: 884 - DNA, 70 bp, Homo sapiens
gccattccattcccagcagctttggagacctccaggattatttctctgtcagccctgccacatatcacta

SEQ ID NO: 885 - DNA, 70 bp, Homo sapiens
gataaaagggggagacaaaagatgtacagaaatgatttcctggctggccaactggtggccagtgggaggt

SEQ ID NO: 886 - DNA, 70 bp, Homo sapiens
caataatcagtggtgcttttgtacctaggttttatgtgattttaatgaaacatggatagttgtggccacc

SEQ ID NO: 887 - DNA, 70 bp, Homo sapiens
tacactgctgtacccagatgcctacaaccatccctgccacatacaggtgctcaataaacacttgtagagc

SEQ ID NO: 888 - DNA, 70 bp, Homo sapiens
tcgggaactggcccaacaggtgcagcaagtagctgctgaatattgtagagcatgtcgcttgaagtctact

SEQ ID NO: 889 - DNA, 70 bp, Homo sapiens
taactctgggaggggctcgagagggctggtccttatttatttaacttcacccgagttcctctgggtttct

SEQ ID NO: 890 - DNA, 70 bp, Homo sapiens
gattaagctgaagatgtttattacaatcactctctgtggggggtggccctgctgctcctcagaatcctgg

SEQ ID NO: 891 - DNA, 70 bp, Homo sapiens
catctacccctgctagaaggttacagtgtattatgtagcatgcaaatgtgtttatgtagtggcttaataa

SEQ ID NO: 892 - DNA, 70 bp, Homo sapiens
ccgctgtcgccgccgcggagacaaagatggctgcgagagtcggcgccttcctcaagaatgcctgggacaa

SEQ ID NO: 893 - DNA, 70 bp, Homo sapiens
ttccatgggagatgactcttaagccataggggctggttttccgtactccaaaccatcaggtggacacagt

SEQ ID NO: 894 - DNA, 70 bp, Homo sapiens
attgtttttatctggttacatatatatttctttgtctaatttaatatgtcaaataaatgagttcatctaa

SEQ ID NO: 895 - DNA, 70 bp, Homo sapiens
tctgcgtgggtggtgatgggggttcacctgaacacagagtgtattttcttattgaggccctgtaccttct

SEQ ID NO: 896 - DNA, 70 bp, Homo sapiens
gaatacatttctgcctgataatcatgctgggttctaataagccctacttccacctaatctgtttacagtc

SEQ ID NO: 897 - DNA, 70 bp, Homo sapiens
ggcccagaagaaatttaagcgtcttatgctgcatcggataaagtgggatgaacagacatctaacacaaag

SEQ ID NO: 898 - DNA, 70 bp, Homo sapiens
gccgagtgtattataaaatcgtgggggagatgcccggcctgggatgctgtttggagacggaataaatgtt

SEQ ID NO: 899 - DNA, 70 bp, Homo sapiens
gcagcgcctcccttgtctcagatggtgtgtccagcactcgattgttgtaaactgttgttttgtatgagcg

SEQ ID NO: 900 - DNA, 70 bp, Homo sapiens
gcatacaggttattggagaaattttccttttgttgcatttgtggaagttagttttctggcccgtggcctt

SEQ ID NO: 901 - DNA, 70 bp, Homo sapiens
ttggcgtagccatggcgtctcgtgtcctttcagcctatgtcagccgcctgcccgcggcctttgcgccgct

SEQ ID NO: 902 - DNA, 70 bp, Homo sapiens
cttcaaatatggccgccaagctccgttctcttttaccgcctgatctacggctacaattctggcttcatgc

SEQ ID NO: 903 - DNA, 70 bp, Homo sapiens
agtgtgtcaaacagatctgcgtggtcatgttggagactctctcccagtcccccccgaagggcgtgaccat

SEQ ID NO: 904 - DNA, 70 bp, Homo sapiens
gggccagggctggatggacagacacctccccctacccatatccctcccgtgtgtggttggaaaacttttg

SEQ ID NO: 905 - DNA, 70 bp, Homo sapiens
cctacttcttcagctgacaccccgtgagccttgtcagtgtgtaaataaagctcttttgccaccccccaaa

SEQ ID NO: 906 - DNA, 70 bp, Homo sapiens
ggcccaacacaattcttcttccaacgtggcccagagaagccaaaagattggatacgcatcagacagatgg

SEQ ID NO: 907 - DNA, 70 bp, Homo sapiens
atcccaacgatgacaaggacagtggcttctttccccgaaacccatcgagctccagcatgaactcggttct

SEQ ID NO: 908 - DNA, 70 bp, Homo sapiens
ttcctcgggcatcgacgtgctcatttccaaagatgatggtgcaggtgaccttttccatcgtgagctaaga

SEQ ID NO: 909 - DNA, 70 bp, Homo sapiens
aaaggttttcacaccagacactgcagcagacacccatgataagtaccatgactccaatgagtgcccaggg

SEQ ID NO: 910 - DNA, 70 bp, Homo sapiens; identical to SEQ ID NO: 307

SEQ ID NO: 911 - DNA, 70 bp, Homo sapiens
gaccataggatgggaggatagggagcccctcatgactgagggcagaagaaattgctagaagtcagaacag

SEQ ID NO: 912 - DNA, 70 bp, Homo sapiens
actactctctgaaggagtccaccactagtgagcagagtgccaggatgacagccatggacaatgccagcaa

SEQ ID NO: 913 - DNA, 70 bp, Homo sapiens
agccgggcgagcgctgtgggccaagcaggggttgcagggtagtaggagtgcagactgaaaaaatgcagac

SEQ ID NO: 914 - DNA, 70 bp, Homo sapiens
gccccagcggtaaccaccaatcttcttttgccaatagacctcgaaaatcatcagtaaatgggtcatcagc

SEQ ID NO: 915 - DNA, 70 bp, Homo sapiens
ctagttatgatcagagcagttactctcagcagaacacctatgggcaaccgagcagctatggacagcagag

SEQ ID NO: 916 - DNA, 70 bp, Homo sapiens
aaaaatgtataatataaaattgtaatacactcaaatgattataaaagtaaaagttggtaatttaggcaaa

SEQ ID NO: 917 - DNA, 70 bp, Homo sapiens
actacctttttcgagagtgactcccgttgtcccaaggcttcccagagcgaacctgtgcggctgcaggcac

SEQ ID NO: 918 - DNA, 70 bp, Homo sapiens
ggtgaacctatgggtcgtggaacaaaagttatcctacacctgaaagaagaccaaactgagtacttggagg

SEQ ID NO: 919 - DNA, 70 bp, Homo sapiens
ggggaagcatttgactatctggaacttgtgtgtgcctcctcaggtatggcagtgactcacctggttttaa

SEQ ID NO: 920 - DNA, 70 bp, Homo sapiens
agcaggctgtgcagagcgcgttgaccaagactcataccagagggccacacttttcaagtgtatatggtaa

SEQ ID NO: 921 - DNA, 70 bp, Homo sapiens
ctcggacgggactttcttggtgcggcagagggtgaaggatgcagcagaatttgccatcagcattaaatat

SEQ ID NO: 922 - DNA, 70 bp, Homo sapiens
cttcaggttcctcttactatgataatgtccggcctctggcctatcctgattctgatgctgtgctcatctg

SEQ ID NO: 923 - DNA, 70 bp, Homo sapiens
cactgtgtaccccgagcaacattctaagggtgtgctttcgccttggctaactcctttgacctcattcttc

SEQ ID NO: 924 - DNA, 70 bp, Homo sapiens
gaatctaagttaccatcccttggaaattctggagaaggagtctcatgcaccacctatcacactccctcac

SEQ ID NO: 925 - DNA, 70 bp, Homo sapiens
gccaggattgctacagttgtgattggaggagttgtggccatggcggctgtgcccatggtgctcagtgcca

SEQ ID NO: 926 - DNA, 70 bp, Homo sapiens
gtcttcaactggttagtgtgaaatagttctgccacctctgacgcaccactgccaatgctgtacgtactgc

SEQ ID NO: 927 - DNA, 70 bp, Homo sapiens
caagaggagagtgaagaggaagaggtcgatgaaacaggtgtagaagttaaggacatagaattggtcatgt

SEQ ID NO: 928 - DNA, 70 bp, Homo sapiens
caaggtgcagaatggtttggaaagtagctgtattcctcagtgtggccctgggcattggtgccattcctat

SEQ ID NO: 929 - DNA, 70 bp, Homo sapiens
cctcgtcagcagcgaggaaggaaacagcggcgacagccctgtactgtgtctgaaattttccatttttgtt

SEQ ID NO: 930 - DNA, 70 bp, Homo sapiens
atgtacacacgtgcacgtacacacatgcatgctcgctaagcggaaggaagttgtagattgcttccttcat

SEQ ID NO: 931 - DNA, 70 bp, Homo sapiens
aacaaaccctcatctcatgaaggacggggtgtgtgtgtggcgttgatctttagcctgtctcacaccagtt

SEQ ID NO: 932 - DNA, 70 bp, Homo sapiens
aattttctgcagcattaaagctggcgcttaataagaataagtaataataaagaaatttctaacattccaa

SEQ ID NO: 933 - DNA, 70 bp, Homo sapiens
gcctggaacaaggaccgcacccagattgccatctgccccaacaaccatgaggtgcatatctatgaaaaga

SEQ ID NO: 934 - DNA, 70 bp, Homo sapiens
ggagtgcttccatccctctccaccccttccccccaaaaggttttctttgcaagtgcttttggaactaaga

SEQ ID NO: 935 - DNA, 70 bp, Homo sapiens
agcagctgcctcaccgcccagacattgatttgttcagatgtttcaatgcctcatgatacaataaaaccac

SEQ ID NO: 936 - DNA, 70 bp, Homo sapiens
agaacaggttttcaaagtggcctcctcagacctggtcaacatgggcatcagtgtggttagctacactctg

SEQ ID NO: 937 - DNA, 70 bp, Homo sapiens
ttctctgctggtaattcctgaagaggcatgactgcttttctcagccccaagcctctagtctgggtgtgta

SEQ ID NO: 938 - DNA, 70 bp, Homo sapiens
gagaccagcctggagcctagatctggtgcttcttctgtgctgtggtttaccccaaacctttaggttgttt

SEQ ID NO: 939 - DNA, 70 bp, Homo sapiens
ggatgggaatagcaatgtgtgttcagagagaatgacaatgtgtgttcagagagaatgaattgcttaaact

SEQ ID NO: 940 - DNA, 70 bp, Homo sapiens
acgcatttgagcgattgctctgtgaagagttgtacactgaacactttcaggggaggctgtttacccaggc

SEQ ID NO: 941 - DNA, 70 bp, Homo sapiens
tgactctctgaggctcattttgcagttgttgaaattgtccccgcagttttcaatcatgtctgaaccaatc

SEQ ID NO: 942 - DNA, 70 bp, Homo sapiens
cggaggtggtcaaggctaaagccggagcaggctctgccaccctctccatggcgtatgccggcgcccgctt

SEQ ID NO: 943 - DNA, 70 bp, Homo sapiens
ctgggtcctggggcagggcgagtccaagtgtgaggctgttgatttgttttcaatatttcttttcgtgctg

SEQ ID NO: 944 - DNA, 70 bp, Homo sapiens
cttaagccttccaggacactaaggtcgtgggagcgggactgcaacaagcaatgccagataactgagaaat

SEQ ID NO: 945 - DNA, 70 bp, Homo sapiens
tatttatcccttcttgcctgtgaggactgcggcttttcgctgtggctcgtccttaacgtttctgaaccac

SEQ ID NO: 946 - DNA, 70 bp, Homo sapiens; identical to SEQ ID NO: 230

SEQ ID NO: 947 - DNA, 70 bp, Homo sapiens
gcagcccctttccgggacacctgggttcacacagctttttagcttacataactggtgcagattttctgtg

SEQ ID NO: 948 - DNA, 70 bp, Homo sapiens
gcaaaatgaattcctggcttcagttagctattatttttttaatgacaacatagactgtgctctaagttta

SEQ ID NO: 949 - DNA, 70 bp, Homo sapiens
aatgcaagctcaccaaggtcccctctcagtccccttccctacaccctgaccggccactgccgcacaccca

SEQ ID NO: 950 - DNA, 70 bp, Homo sapiens
tatgatgtatttctgagctaaaactcaactatagaagacattaaaagaaatcgtattcttgccaagtaac

SEQ ID NO: 951 - DNA, 70 bp, Homo sapiens
attttacctctttaccctgtcgctcataatgaggcatcatatatcctctcactctctgggacaccatagc

SEQ ID NO: 952 - DNA, 70 bp, Homo sapiens
gacacctatctaagccattttaaccctcgggattacctagaaaaatattacaagtttggttctaggcact

SEQ ID NO: 953 - DNA, 70 bp, Homo sapiens
attgaaagctaagtgagagagccagagggcctccttggtggtaaaagagggttgcatttcttgcagccag

SEQ ID NO: 954 - DNA, 70 bp, Homo sapiens
acattcacatctagtcaagggcataggaacggtgtcatggagtccaaataaagtggatattcctgctcgg

SEQ ID NO: 955 - DNA, 70 bp, Homo sapiens
caagggcgcaagagtagcggtccaagcctgcaactcatctttcattaaaggcttctctctcaccagcaaa

SEQ ID NO: 956 - DNA, 70 bp, Homo sapiens
agcaccgccgcggagaacaaggccagccccgcggggacagcggggggacctggggctggagcagctgctg

SEQ ID NO: 957 - DNA, 70 bp, Homo sapiens
ctagaagactgcaggctggatcatgctttatatgcactgcctgggccaaccatcgtggacctgaggaaaa

SEQ ID NO: 958 - DNA, 70 bp, Homo sapiens
gagaaatcgaatattctggagcactgattgcagcagggtggctcctttgtgtgcagcaggtgtagtagtc

SEQ ID NO: 959 - DNA, 70 bp, Homo sapiens
cactgctgttgtcattgctccgtttgtgtttgtactaatcagtaataaaggtttagaagtttgaccctaa

SEQ ID NO: 960 - DNA, 70 bp, Homo sapiens
ctcggacaatttctgggtggtgactgagtacccctttagtgagtacccctttagtgctatatttgtgcca

SEQ ID NO: 961 - DNA, 70 bp, Homo sapiens; identical to SEQ ID NO: 415

SEQ ID NO: 962 - DNA, 70 bp, Homo sapiens
gtatgttcaccaggggaatggctgggatttctcggcactctgcatcatccatcttttcttataggtggga

SEQ ID NO: 963 - DNA, 70 bp, Homo sapiens
cctcattcccttttttctttacccaggattggtttcttcaataaatagataagatcgaatccatttaaaa

SEQ ID NO: 964 - DNA, 70 bp, Homo sapiens
cagtggccatcatcctcccgccaggagcttcttcgttcctgcgcatatagactgtacgttatgaagaata

SEQ ID NO: 965 - DNA, 70 bp, Homo sapiens
agcacaagcagttggagcttccacccctacgaccagtagcccagcacctgcagtatccacttcaacatca

SEQ ID NO: 966 - DNA, 70 bp, Homo sapiens
gcctatcacctccagcacaatcccagcgaaaaaggtgtgaagcacccaccatgttcttgaacaatcaggt

SEQ ID NO: 967 - DNA, 70 bp, Homo sapiens
gggaacagtggtactaacccacgattctgagccctgagtatgcctggacattgatgctaacatgacatgc

SEQ ID NO: 968 - DNA, 70 bp, Homo sapiens
aacagaagccgcagtcccgtggggtctggagacgcagtttccttgttaatgacaataaatccctgctccc

SEQ ID NO: 969 - DNA, 70 bp, Homo sapiens
ctgccacagggcccttcctacctttggatctgtgagaaggtgaatacaaagcagcaggcagagtaaaatc

SEQ ID NO: 970 - DNA, 70 bp, Homo sapiens
ttcccacatgccgtgactctggactatatcagtttttggaaagcagggttcctctgcctgctaacaagcc

SEQ ID NO: 971 - DNA, 70 bp, Homo sapiens
cttcctctttccctcggagcgggcggcggcgttggcggcttgtgcagcaatggccaagatcaaggctcga

SEQ ID NO: 972 - DNA, 70 bp, Homo sapiens
tcctccactataagtctaatgttctgactctctcctggtgctcaataaatatctaatcataacagcaaaa

SEQ ID NO: 973 - DNA, 70 bp, Homo sapiens
gaatcgacgtctcaagaggttctccatggtggtacaggatggcatagtgaaggccctgaatgtggaacca

SEQ ID NO: 974 - DNA, 70 bp, Homo sapiens
cccctgtccccactcgcgttccgcatggaggatactgaggccttacccctaaccccgatcctctacccaa

SEQ ID NO: 975 - DNA, 70 bp, Homo sapiens
atcactgtaaatggtaatcagttggaattctcctaaatgtcttccagacactagtaaaaaacgacctgaa

SEQ ID NO: 976 - DNA, 70 bp, Homo sapiens
gcaggaaaactagcatgaaatattgtttcaggccctgggttctatgtgacactacattaggaattggatt

SEQ ID NO: 977 - DNA, 70 bp, Homo sapiens
gaaaatcgggttcacaggctccacagaggtgggcaagcacatcatgaaaagctgtgccataagtaacgtg

SEQ ID NO: 978 - DNA, 70 bp, Homo sapiens
gagtgattctgatatatgtacttgtcacattggtgttggacacatttgcgccaaaagtatggtaattcta

SEQ ID NO: 979 - DNA, 70 bp, Homo sapiens
ggcatggcagtacccatgttgatttgacatctctctagcccatccattgcttacagtagaagagtggggc

SEQ ID NO: 980 - DNA, 70 bp, Homo sapiens
gcctctcagtcttaggggacatggcagagatgaaagaaagaaagagtgggtttcagaagtgtcagggtgg

SEQ ID NO: 981 - DNA, 70 bp, Homo sapiens
gatgcggggcctggcggtcttcatctcggatatccgcaactgtaaaagtaaagaagcagaaataaaaagg

SEQ ID NO: 982 - DNA, 70 bp, Homo sapiens
ccacctggtcatatactctgcagctgttagaatgtgcaagcacttggggacagcatgagcttgctgttgt

SEQ ID NO: 983 - DNA, 70 bp, Homo sapiens
attgaacatggtcttgtggatgagcagcagaaagttcggaccatcagtgctttggccattgctgccttgg

SEQ ID NO: 984 - DNA, 70 bp, Homo sapiens
gagggcagtaggccatcccccaggagaatgacagaagcaaaggacttgttactaagcagatttaagggtc

SEQ ID NO: 985 - DNA, 70 bp, Homo sapiens
cccccctctgaattttactgatgaagaaactgaggccacagagctaaagtgacttttcccaaggtcgccc

SEQ ID NO: 986 - DNA, 70 bp, Homo sapiens
aatgttgctgatagggataaatcttgaggctgagggcgggtggtacagatgtgtatgggaaaccccaacc

SEQ ID NO: 987 - DNA, 70 bp, Homo sapiens
cgtgctgcctctcttctgtgtcgttttgttgccaaggcagaatgaaaagtccttaaccgtggactcttcc

SEQ ID NO: 988 - DNA, 70 bp, Homo sapiens
ggccaggcgcgctctgcccagcccagcctacagtgcggataaaggtgcggatgctgctggccctgaaaaa

SEQ ID NO: 989 - DNA, 70 bp, Homo sapiens
agatctgctgcctcgcctctagatatggtgccctggtcttcatggatgaatgccatgccactggcttcct

SEQ ID NO: 990 - DNA, 70 bp, Homo sapiens
cccaagtgaagagaacgtcatgagtgtaagtgcaaatcagtggaaggagcggcaaactgggacatgcaga

SEQ ID NO: 991 - DNA, 70 bp, Homo sapiens
tgtggagggcgagctgagccctggccgccgccacaatgggccgcgagtttgggaatctgacgcggatgcg

SEQ ID NO: 992 - DNA, 70 bp, Homo sapiens
ccccctgaagtcaggaccagtgcctgtgatctccattactttattttcctggaggtattagccaacacag

SEQ ID NO: 993 - DNA, 70 bp, Homo sapiens
tgggaggcgggcgcagggtagctgttggcgccgccgcgtttctgggcctggccaactcacgtgaccgacg

SEQ ID NO: 994 - DNA, 70 bp, Homo sapiens
ggaaagacctgcccccgtgattaaattatttcccaccaggcccctcccacaacatggaataatgggagat

SEQ ID NO: 995 - DNA, 70 bp, Homo sapiens
ggtgtggattattgggccaaaagaggaagaggtcgtggtacttttcaacgtggcagagggcgctttaact

SEQ ID NO: 996 - DNA, 70 bp, Homo sapiens
ctgcccttggtgcattagcaagggtcctgagagaagactggaagcaaagtgtcgagttagctacaaacat

SEQ ID NO: 997 - DNA, 70 bp, Homo sapiens
atcgagatgccaagaagggctatggaactatgcaggtggctagtggtcagactgaagtcaccagctgaat

SEQ ID NO: 998 - DNA, 70 bp, Homo sapiens
catcatacaaaccacattacttctgtcacttcagggcatcgggactggctggcgcccttgttatgtgcta

SEQ ID NO: 999 - DNA, 70 bp, Homo sapiens
tcactcgcccagtcttcagtctcctgacttagagatacaatcacgtcacaggtctcttggcctcaatctg

SEQ ID NO: 1000 - DNA, 70 bp, Homo sapiens
aggggctgctgtccacagcttggggctgaagactcccaggccattaaccccttagcttttaggaagatta

SEQ ID NO: 1001 - DNA, 70 bp, Homo sapiens
ctcacgctgatggcttggcagagcaccttcggttaacttgcatctccagattgattactcaagcagacag

SEQ ID NO: 1002 - DNA, 70 bp, Homo sapiens
cgagtggtctgtgttcctattgctggtggggtgatagggtgggctaaaaaccatgcactctggaatttgt

SEQ ID NO: 1003 - DNA, 70 bp, Homo sapiens
gaggtgctcaataagcaaaagtggtcggtggctgctgtattggacagcacagaaaaagatttccatcacc

SEQ ID NO: 1004 - DNA, 70 bp, Homo sapiens
attactgtggagcagctttcattcctacccacttgcaaaccttggcgctgttgtctgagattgctgcagc

SEQ ID NO: 1005 - DNA, 70 bp, Homo sapiens
tggacagtgcaatgaaggaagaagtgcagaggctgcagtccagggtggacctgctggaggagaagctgca

SEQ ID NO: 1006 - DNA, 70 bp, Homo sapiens
tcgctcaagctttcgaagacacatgatggcacacactggagatggccctcataaatgcacagtatgtggg

SEQ ID NO: 1007 - DNA, 70 bp, Homo sapiens
acccaatgtggacttcttttaaacctttctaatgcccataacccagcctcagacccatggagcccacgag

SEQ ID NO: 1008 - DNA, 70 bp, Homo sapiens
aggtcctctgaggatcagatcatgcatgcgccattttttacttaatgcagctgttaaattggcaaagctc

SEQ ID NO: 1009 - DNA, 70 bp, Homo sapiens
cagcccataagagacattctcagatgaaactctgttttcttgccccagtcaggctcaagccctgtggttg

SEQ ID NO: 1010 - DNA, 70 bp, Homo sapiens
gctctgtatgtcctcaggggactgacaacatcctccagattccagccataaaccaataactaggctggac

SEQ ID NO: 1011 - DNA, 70 bp, Homo sapiens
aattccagtggcaaaaattcgaacagaacaggaaagcaaaggccctatgacccgccgactgctgctgcat

SEQ ID NO: 1012 - DNA, 70 bp, Homo sapiens
ccaatactttagaagtttggtcgtgtcgtttgtatgaaaatctgaggctttggtttaaatctttccttgt

SEQ ID NO: 1013 - DNA, 70 bp, Homo sapiens
ttttctagagcaaagcaaagtagcttcgggtcttgatgcttgagtagagtgaagaggggagcacgtgccc

SEQ ID NO: 1014 - DNA, 70 bp, Homo sapiens
gctctaggccctcacctcaaaccttgccattggttgccgtatttcaaggtcaatatagtttccctcactt

SEQ ID NO: 1015 - DNA, 70 bp, Homo sapiens
gctccattaaatagccgtagacggaacttcgcctttctctcggccttagcgccatttttttggaaacctc

SEQ ID NO: 1016 - DNA, 70 bp, Homo sapiens
tgacaacgaaggccgcgcctgcctttcccatctgtctatctatctggctggcagggaaggaaagaacttg

SEQ ID NO: 1017 - DNA, 70 bp, Homo sapiens
ggggagcacatattggatgtatatgttaccatatgttaggaaataaaattattttgctgaaacttggaaa

SEQ ID NO: 1018 - DNA, 70 bp, Homo sapiens
ctttggatccatttcatgcaggattgtgttgttttaactgttgttgaggaagctaataaataattaaatt

SEQ ID NO: 1019 - DNA, 70 bp, Homo sapiens
gaacccaatggtagtcttaaagagttttgtgccctggctctatggcggggaaagccctagtctatggagt

SEQ ID NO: 1020 - DNA, 70 bp, Homo sapiens
ccttctccaacatacatcctgcattacatgaatggattattcctaataattaataaaaaggtattttttc

SEQ ID NO: 1021 - DNA, 70 bp, Homo sapiens
gacaacacaaaactagagccaggggcctccgtgaactcccagagcatgcctgatagaaactcatttctac

SEQ ID NO: 1022 - DNA, 70 bp, Homo sapiens
gcacagagtcaggatctcacatttcaccccaggctcaactgaggatgtggcttattaaacacggaagtgc

SEQ ID NO: 1023 - DNA, 70 bp, Homo sapiens
tcccgtgcaacagcagaatcaaattggatatccccaaccttatggccagtggggccagtggtatggaaat

SEQ ID NO: 1024 - DNA, 70 bp, Homo sapiens
gctcccacggaggggagcaggaatgctgcactgtttacaccctgactgtgcttaaaaacactttcactaa

SEQ ID NO: 1025 - DNA, 70 bp, Homo sapiens
taaaaatacaaaaattagccgggcgtggtggcttacgcctgtaatcccagcactttgggaggccaaggtg

SEQ ID NO: 1026 - DNA, 70 bp, Homo sapiens
tggctgtgcttattgccctcaccatttatgacgaagatgtgttggctgtggaacatgtgctgaccaccgt

SEQ ID NO: 1027 - DNA, 70 bp, Homo sapiens
ggaaaagcattggcacgcaacgcagcatgtggcttcattgaggcagttgatggagttaaaccatctgctc

SEQ ID NO: 1028 - DNA, 70 bp, Homo sapiens
cgtgccttcttgctgtcatgcaatgaccccgccttatgttgccgaaataagcaactcttaggtttgcctg

SEQ ID NO: 1029 - DNA, 70 bp, Homo sapiens
gagttttcctcggaaacactcttgaatgtctgagtgagggtcctgcttagctctttggcctgtgagatgc

SEQ ID NO: 1030 - DNA, 70 bp, Homo sapiens
gaggcagaatggctctgctgagcctcctacccatgacaacaccccaataaacagaacattcagagccaaa

SEQ ID NO: 1031 - DNA, 70 bp, Homo sapiens
gggttttcctgggagcgaatatcaagtgcctgagagcaactacaggactaactgtgtttgggttgggtgt

SEQ ID NO: 1032 - DNA, 70 bp, Homo sapiens
ggaaccccaggttcgcggcccgtgtttccgaccggcggagggggctcagcggcccgatcccacggaagcg

SEQ ID NO: 1033 - DNA, 70 bp, Homo sapiens
tctccagatgaggttgcaaggaccaaccagtgcctacccgcccatgctcccccgaaactgggaactgaca

SEQ ID NO: 1034 - DNA, 70 bp, Homo sapiens
ccctgctattagaccaccccctcatggcacaactgcccctcacaagaattcagcttcagtgcaaaattca

SEQ ID NO: 1035 - DNA, 70 bp, Homo sapiens
acattcttcctttgcatttgctggtctggcctttgcgtccttctacctggcagggaagttacactgcttc

SEQ ID NO: 1036 - DNA, 70 bp, Homo sapiens
acaggggaagatcccgagtgcaagaaagagacaaagagcccctacaggaacgctttttccgaccacattt

SEQ ID NO: 1037 - DNA, 70 bp, Homo sapiens
gggcagtcgctgcagggagcaccacggccagaagtaacttattttgtactagtgtccgcataagaaaaag

SEQ ID NO: 1038 - DNA, 70 bp, Homo sapiens
gctgcaatgatgttagctgtggccactgtggatttttcgcaagaacattaataaactaaaaacttcatgt

SEQ ID NO: 1039 - DNA, 70 bp, Homo sapiens
aattcatgacccacaaacttaaacatactgagaatactttcagccgccctggagggagggccagcgtgga

SEQ ID NO: 1040 - DNA, 70 bp, Homo sapiens
agttggtcgggatcctgctcagcgccctgctaggggttgccctgggacaccgcacgcggtgctatgactg

SEQ ID NO: 1041 - DNA, 70 bp, Homo sapiens
gataatatctctcacccggatccctcctcacttgccctgccactttgcatggtttgattttgacctggtc

SEQ ID NO: 1042 - DNA, 70 bp, Homo sapiens
tggccgccatgaggaaagctgctgccaagaaagactgagcccctcccctgccctctccctgaaataaaga

SEQ ID NO: 1043 - DNA, 70 bp, Homo sapiens
caaggccagtagaaagctatggctgcaaaaccctggggtggacgatgtttgatgattagacggtcatctc

SEQ ID NO: 1044 - DNA, 70 bp, Homo sapiens
cccaggagtttgaggccagcctgggcaacatggtgaaacccggtgtctaccaaaaatacaaaatgtatcc

SEQ ID NO: 1045 - DNA, 70 bp, Homo sapiens
ctgtttttctgtatgctctgtgctagtagggtggattcagtaataaatatgtgaaagcttttgtttccaa

SEQ ID NO: 1046 - DNA, 70 bp, Homo sapiens
tgaattctacaaccggttcaagggccgcaatgacctgatggagtacgcaaagcaacacgggattcccatc

SEQ ID NO: 1047 - DNA, 70 bp, Homo sapiens
cgctgtaaaactccgaaatctggcacaaacccaacacggagctacgcaatactgctggagagcatttgct

SEQ ID NO: 1048 - DNA, 70 bp, Homo sapiens
acttcaccgaagaccagaccgcagatctgatcccaagcactgagttcaaggaggccttccagctgtttga

SEQ ID NO: 1049 - DNA, 70 bp, Homo sapiens
cgaggcctggggagatgttgttttcatgctgcttccaccatcacactggggtttctggatgggaaataaa

SEQ ID NO: 1050 - DNA, 70 bp, Homo sapiens
cacgcagccatggttgtgcctgccgttcatggtggtctttcaggttatcttggcaacatgtacattgctt

SEQ ID NO: 1051 - DNA, 70 bp, Homo sapiens
gtgtggctgcggttgggtatggatcaagcaagggttcagattacatcattgtgaagaattcttggggacc

SEQ ID NO: 1052 - DNA, 70 bp, Homo sapiens
tgccttctaaatgtggtgtcgatctcccttacaagttcagcccttccactgactgcgacagtatccagtg

SEQ ID NO: 1053 - DNA, 70 bp, Homo sapiens
acgggcttatgatccctcgagcactatttatccgtgatttgatgtggctcactggttcgctatgggcaac

SEQ ID NO: 1054 - DNA, 70 bp, Homo sapiens
cgccctgaaggagtacatcgtctagtgagggacagaccaagcacgcaaaacaaattgcaatataatgtga

SEQ ID NO: 1055 - DNA, 70 bp, Homo sapiens
gctcatttgagataaagtcaaatgccaaacactagctctgtattaatccccatcattactggtaaagcct

SEQ ID NO: 1056 - DNA, 70 bp, Homo sapiens
tctttccttctgatctgagaagacatgaacgttttctcttcaccgccgtggggtgtattgactggtcccc

SEQ ID NO: 1057 - DNA, 70 bp, Homo sapiens
ctttcccagaagatggaggagagtatatgtgtaaagcagtcaacaataaaggatctgcagctagtacctg

SEQ ID NO: 1058 - DNA, 70 bp, Homo sapiens
tcacattttcccaaaaaaagttgatctctcccagtgggctgtaggcagggtcctccatgggtttccaacc

SEQ ID NO: 1059 - DNA, 70 bp, Homo sapiens
aaaattccagagtgaccgtggcacttgggtgtacaggtaattcctccagagctgtttgctggcttcagga

SEQ ID NO: 1060 - DNA, 70 bp, Homo sapiens
gtggggaagagctattgtaggctccccctcctctgacttatgtaatcaaagccacttttgtgtgtgtcta

SEQ ID NO: 1061 - DNA, 70 bp, Homo sapiens
tcatcttgcttgggcttaccaaatgcattagtctttgtgtttgggtcgacagcgagtgtgcctgtgctgg

SEQ ID NO: 1062 - DNA, 70 bp, Homo sapiens
ctgttcttgtttcaaagcaccacttggaggctgcggaagatacccgtgtaaaggaaccactgtcttcagc

SEQ ID NO: 1063 - DNA, 70 bp, Homo sapiens
cctctgcagtccgtgggctggcagtttgttgatcttttaagtttccttccctacccagtccccattttct

SEQ ID NO: 1064 - DNA, 70 bp, Homo sapiens
gtgccacttcatggtgcgaagtgaacactgtagtcttgttgttttcccaaagagaactccgtatgttctc

SEQ ID NO: 1065 - DNA, 70 bp, Homo sapiens
aaccctccataaacctggagtgactatatggatgccccccaccctaccacacattcgaagaacccgtata

SEQ ID NO: 1066 - DNA, 70 bp, Homo sapiens; identical to SEQ ID NO: 594

SEQ ID NO: 1067 - DNA, 70 bp, Homo sapiens; identical to SEQ ID NO: 621

SEQ ID NO: 1068 - DNA, 70 bp, Homo sapiens
tcgcccacgggcttacatcctcattactattctgcctagcaaactcaaactacgaacgcactcacagtcg

SEQ ID NO: 1069 - DNA, 70 bp, Homo sapiens
cttcccacctcagcctcctgaatagctgggactaccagcacgctccaccatgccttgctaattatttttt

SEQ ID NO: 1070 - DNA, 70 bp, Homo sapiens
ttgctactttggcaaaaactagcgaggggtagcagaaacctgcaccaaggattgtccctatgtcttggcc

SEQ ID NO: 1071 - DNA, 70 bp, Homo sapiens
tatcaataaagttgctcacttgttgccggcccgctagcccgaaaggttgcgcgcgcagaccgagaagtct

SEQ ID NO: 1072 - DNA, 70 bp, Homo sapiens
aagattgatggaagcctcgggcctaaagaatcacagagttatggagaaggtagctcggagagcctcctga

SEQ ID NO: 1073 - DNA, 70 bp, Homo sapiens
actaaccctatgttgcacacgctgggttcctgatcttggtgcgatgttttggttacatggcatctggcag

SEQ ID NO: 1074 - DNA, 70 bp, Homo sapiens
taaagataaagccagaagctaagctgcagtgaggctgtgattgggcgtagaagtgggagcattgggacct

SEQ ID NO: 1075 - DNA, 70 bp, Homo sapiens
acaaccagaaggcccttaactatcaccagtgcatcacatctgcacactctcttctccattccctagcagg

SEQ ID NO: 1076 - DNA, 70 bp, Homo sapiens
cagtatccaaaaatagccctgcaaaaattcagagtccttgcaaaattgtctaaaatgtcagtgtttggga

SEQ ID NO: 1077 - DNA, 70 bp, Homo sapiens; identical to SEQ ID NO: 822

SEQ ID NO: 1078 - DNA, 70 bp, Homo sapiens
agagtgtcatggacctgataaagcgaaactccggatgggtgtttgagaatccctcaataggcgtgctgga

SEQ ID NO: 1079 - DNA, 70 bp, Homo sapiens
cagagtgttgggtctgtagccagcaaattacttcatcatctagattatccattcagttgatcctaattag

SEQ ID NO: 1080 - DNA, 70 bp, Homo sapiens
ttgctcaccctcggtaaagagagagagggctgggaggaaaagtagttcatctaggaaactgtcctgggaa

SEQ ID NO: 1081 - DNA, 70 bp, Homo sapiens
ccgctcccacctccctgctgggaaaccacagcattatcacagcattattgtgacagccacgaacccattg

SEQ ID NO: 1082 - DNA, 70 bp, Homo sapiens
ggagaggtaggtgacatagtgctttggagcccagggagggaaaggttctgctgaagttgaattcaagact

SEQ ID NO: 1083 - DNA, 70 bp, Homo sapiens
gccggggcccgaatccaggcactgctgggctgcctgctcaaggtgctgctctgggtggcctctgccttgc

SEQ ID NO: 1084 - DNA, 70 bp, Homo sapiens; identical to SEQ ID NO: 31

SEQ ID NO: 1085 - DNA, 70 bp, Homo sapiens
gaggcgtccagcgagccgccgctggatgctaagtccgatgtcaccaaccagcttgtagattttcagtgga

SEQ ID NO: 1086 - DNA, 70 bp, Homo sapiens
aaacaggagccttacccaggaactcttttttatgccagaacgcttcctctcccctgctgtctctggggct

SEQ ID NO: 1087 - DNA, 70 bp, Homo sapiens
gagcgcggctgcgccggcgcgtcgaggggagaggcagcagccgcgatggacgtgttcctcatgatccggc

SEQ ID NO: 1088 - DNA, 70 bp, Homo sapiens
cggaaaaaattgtattgaaaacacttagtatgcagttgataagaggaatttggtataattatggtgggtg

SEQ ID NO: 1089 - DNA, 70 bp, Homo sapiens
cacggaccaggttcccgcaaaacattgccagctagtgaggcataatttgctcaaagtatagaaacagccc

SEQ ID NO: 1090 - DNA, 70 bp, Homo sapiens
tcagcttccaggaccttggctggctggtaattgctgactctccttgtttctgtgccgcaccacaggcagg

SEQ ID NO: 1091 - DNA, 70 bp, Homo sapiens
ggtagcggccgaggtacactcggcttggctgttggagttgcttgtggcatgtgcctgggctggagccttc

SEQ ID NO: 1092 - DNA, 70 bp, Homo sapiens
tcaaagtatatgtagagatgactattttatattacatgacccaatcctgtatttatttctaccccctttt

SEQ ID NO: 1093 - DNA, 70 bp, Homo sapiens
attaaagttctttttattgcagtttggaaagcatttgtgaaactttctgtttggcacagaaacagtcaaa

SEQ ID NO: 1094 - DNA, 70 bp, Homo sapiens
tcattaagaacttttcaaaagtgaattagtgaggattcagcttaatacctgtatcaaatgaggaagtggt

SEQ ID NO: 1095 - DNA, 70 bp, Homo sapiens
ccccgatcatcgtgcttatctaatacctcacgaccttctctcggcgggccctggtttcctgctgaacgat

SEQ ID NO: 1096 - DNA, 70 bp, Homo sapiens
acatgatgagttggcattagcttctccaggcatgggaacttaacagatgaggttaagaaccgtagacagt

SEQ ID NO: 1097 - DNA, 70 bp, Homo sapiens
catcagaagtgtttcttattattattttatattgagttgaatattgaactctaacagttttctacataca

SEQ ID NO: 1098 - DNA, 70 bp, Homo sapiens
agacataatgtagacatagaggaggaacagctgagagtctctgcatcacagaaagagaaacctgagcaaa

SEQ ID NO: 1099 - DNA, 70 bp, Homo sapiens
aatgtcctagaaacaaatatagaaaaatatattcatgagcttaggagaatgtaggcaaagttttcctggc

SEQ ID NO: 1100 - DNA, 70 bp, Homo sapiens
cggccctgtgtgcctcagggcagatatagcaagctctttcgaccatagttgatggtaggacattttagac

SEQ ID NO: 1101 - DNA, 70 bp, Homo sapiens
ggacattgtatttgatggcatcgctcagatccgtggtgagatcttcttcttcaaggaccggttcatttgg

SEQ ID NO: 1102 - DNA, 70 bp, Homo sapiens
gggatgagggatcatgcatgatcagttaagtcactctgccactttttaaaataatacgattcacatttgc

SEQ ID NO: 1103 - DNA, 70 bp, Homo sapiens
aacatcattctcaccaccagtctcttctctgtgcctttcttcctgacgtggagtgtggtgaactcagtgc

SEQ ID NO: 1104 - DNA, 70 bp, Homo sapiens
ccgcacctggccttccctgcttcctctctagaatccaattagggatgtttgttactactcatattgatta

SEQ ID NO: 1105 - DNA, 70 bp, Homo sapiens
aaggagattgagtacgaggtggtgagagacgcctatggcaactgtgtcacggtgtgtaacatggagaact

SEQ ID NO: 1106 - DNA, 70 bp, Homo sapiens
atgttcaagttccacattggtcttcaactctctggcggggtcagaggaccatctgtgctcgctcagatat

SEQ ID NO: 1107 - DNA, 70 bp, Homo sapiens
acagcggcagtcgcgcccacacgtccatgactggtcgtcctagattttaggtgtcgatgaatacggccca

SEQ ID NO: 1108 - DNA, 70 bp, Homo sapiens
ccttctgtgactccctgcagccactgcttcttgaagcctttgtctctaagcttctgtccagctcaaaccc

SEQ ID NO: 1109 - DNA, 70 bp, Homo sapiens
cggaaacgggaaggcctgctgcattccagccacatctcggaggagctgaccacaactacagagatgatga

SEQ ID NO: 1110 - DNA, 70 bp, Homo sapiens
catgaaaaccatgaaggggccttttggctgaaattccccacctgcctttggatgaaagactccgttggga

SEQ ID NO: 1111 - DNA, 70 bp, Homo sapiens
agaggagcccacgtcgcctgtcacccaatatctccagccgcgcagtcccgaagagtgtaagatgttcgcc

SEQ ID NO: 1112 - DNA, 70 bp, Homo sapiens
gagtatgaaggagagaagagggtactgaccatgcgtttcaacataccaactgggaccaatttaccccctg

SEQ ID NO: 1113 - DNA, 70 bp, Homo sapiens
taaatacatccaaacatgatcatcgttggagccggaggtggcaggagtcgaggcgctgatccctaaaatg

SEQ ID NO: 1114 - DNA, 70 bp, Homo sapiens
gattcctcctttcccccccaaatattaactccagaaactaggcctgactggggacaccctgagagtagta

SEQ ID NO: 1115 - DNA, 70 bp, Homo sapiens
tactaaacataaaaaaattagcctggcatggtggtgtacgcctgtaatcccagtgacttgggaggctgag

SEQ ID NO: 1116 - DNA, 70 bp, Homo sapiens
ggtggagaggaattgccggagctctgaaaatcctaatgaagtgttccgcttcttggtggaggaaaggatc

SEQ ID NO: 1117 - DNA, 70 bp, Homo sapiens
agctgctctatagcaatgtttctaactttgcccgcctggcttccaccttggttcacctcggtgagtatca

SEQ ID NO: 1118 - DNA, 70 bp, Homo sapiens
tcccaacagattgggctgggtgggggttgacaatggggtcagatactaaagggtcagaatttctaagcag

SEQ ID NO: 1119 - DNA, 70 bp, Homo sapiens
agccacatctgcctctgagctgcctgcgtcctctcggtgagctgtgcagtgccggccccagatcctcaca

SEQ ID NO: 1120 - DNA, 70 bp, Homo sapiens
tggcctgcttggcaaggcaagtagcggcggcgcttcaagatgcgctgcctgaccacgcctatgctgctgc

SEQ ID NO: 1121 - DNA, 70 bp, Homo sapiens
gagagcattccgcaaagctgcttgttttccaatttcttcattcttccccttagcactggtgcagctgaat

SEQ ID NO: 1122 - DNA, 70 bp, Homo sapiens
ctggtggccattagtcactcttcatttggctggaactaccgcacggaccctttgaagatatgtgtggatg

SEQ ID NO: 1123 - DNA, 70 bp, Homo sapiens
gccctgggccttaagagccagctcttcctatcctgtagcgtgtagaaaacgtggactcatttcactatgt

SEQ ID NO: 1124 - DNA, 70 bp, Homo sapiens
agattcatatgggctggtgttcctgtgcgctgtgggtgtggtgattcagcctggcatttctaccataagt

SEQ ID NO: 1125 - DNA, 70 bp, Homo sapiens; identical to SEQ ID NO: 1088

SEQ ID NO: 1126 - DNA, 70 bp, Homo sapiens
cgagcccggccccgccagcccagcccagcccagccctactccctccccacgccagggcagcagccgttgc

SEQ ID NO: 1127 - DNA, 70 bp, Homo sapiens
gcttgggtaagtacgcaacttacttttccaccaaagaactgtcaccacctgcctgcttttctgtgatgta

SEQ ID NO: 1128 - DNA, 70 bp, Homo sapiens
ttccctgaggaggcgaatccggcgggtatcagagccatcagaaccgccaccatgacggtgggcaagagca

SEQ ID NO: 1129 - DNA, 70 bp, Homo sapiens
cgacaaggaagatttgcatgatatgcttgcttcattggggaagaatccaactgatgagtatctagatgcc

SEQ ID NO: 1130 - DNA, 70 bp, Homo sapiens
tgattattactgtgcagcatgggatgacagcctgaatggtgtggtattcggcggagggaccaagctgacc

SEQ ID NO: 1131 - DNA, 70 bp, Homo sapiens
ctacagttggaaatccatccagaggccatgttccaataaacaggaggtcgtgtatttggtcacgacattt

SEQ ID NO: 1132 - DNA, 70 bp, Homo sapiens
ctaccgcccagtcactcaaatccgtggactacgaggtgttcggaagagtgcagggtgtttgcttcagaat

SEQ ID NO: 1133 - DNA, 70 bp, Homo sapiens
ctctgaaaaaaatgatttcaaggcatggaagttctctgtgatacaacaatacgtattcttcaaatgcgcc

SEQ ID NO: 1134 - DNA, 70 bp, Homo sapiens
gcccatctcagcaagttccatgtcagccttggcagaagcctctttctttcctcttccccataagagacat

SEQ ID NO: 1135 - DNA, 70 bp, Homo sapiens
ggggacagcgcttgccttggtcagaccttcccacatctacatactctcaaatacatgaccaggtgatcaa

SEQ ID NO: 1136 - DNA, 70 bp, Homo sapiens
gcatctgtaggagacagagtcaccatcacttgccgggcaagtcagagcattagcagctatttaaattggt

SEQ ID NO: 1137 - DNA, 70 bp, Homo sapiens
ggcagcgaactgagtgaaggggaattggaaaagcgcagaagaacccttttggagcaactggatgatgatc

SEQ ID NO: 1138 - DNA, 70 bp, Homo sapiens
atggcctattcacacagatccatcagcgcactgccagcaagcttctcggtcactagaatgagattaaaaa

SEQ ID NO: 1139 - DNA, 70 bp, Homo sapiens
aagctcgcaactgtgtaggatgaattctgtacacttttatttccctctgttctcctttcctatttgaaag

SEQ ID NO: 1140 - DNA, 70 bp, Homo sapiens
aggagcgtcggtagttcttgcagtaggcactttatcaggacctgacctgttgctgggtgattttagtctc

SEQ ID NO: 1141 - DNA, 70 bp, Homo sapiens
tcgcacgaggatgcttggcacgtaccccgtctacatacttcccaggcacccagcatggaaataaagcacc

SEQ ID NO: 1142 - DNA, 70 bp, Homo sapiens
taggagattttcattttgtgtgactcccatggggaggaacagactggcaggaagcacaccggggttaaca

SEQ ID NO: 1143 - DNA, 70 bp, Homo sapiens
cactccaacccaaactagctgggagttcagaaccatggtggaataaagaaatgtgcatctgctcgtgccg

SEQ ID NO: 1144 - DNA, 798 bp, Gallus gallus
aggtaccaataaaacgtaggcttttgactttaggaaaatacaagaaattttaatgcaacagacaggagagaggtccagcatagatatctaacgtgttagttttatttaaagatggtctcacggtgcyagagtaagaaatgttattaagaaaacgagagagagagggagagagatcaaataaataaataaataaataaataaaaataggaataactttctgttgacgagctttcatcttgggaggaacgggttctgtgaagcattcttcagagtgaagtggtcctaattcttcctggaaccattgcaacccattccactcagggagccaatcctatcaattcttctgccgaagcagccagaatctctcatcatccggggcatctgcacccccctcagtctcttgaggaaggggttcctgtaggacagaggagtgttggatgctagcttgggttcagccttctgctcatcgctgtcatctatgagttctggtggaatctcctctcgggtttggggctcttgtaggtcaggattggactccagggcttcaatcagtgcgaatttgtcctccagtctctccagaagagcctccatgctggccagttctttggcagggctgaggttgtagatggggttggccctgctgggctgcagctggacaagaagcagcaagaggaaaccacaggaaaatgagcctctagtgtccatggcgctgggttcgttgggaatatgggaagttcaagctgtttcttctgagatggctcttcaggtctctctcttsgckgggaccasgctaatcas

SEQ ID NO: 1145 - DNA, 367 bp, Gallus gallus
ctgattagcctggtccccgcccaggtactccaagggacagaagaggagctacagataaagagaaactgccagctaaagctgtatatgactttaaagctcaaacttctaaagagctgtcctttaagaaaggagatactgtctatatcctcaggaaaattgatcaaaactggtatgaaggtgagcaccatggaagagttggaatattcccaatttcttatgttgagaaactttctcctccagaaaaagcacagyctgccaggccgcctccccctgcacagattggagagattggagaagctattgccaaatataatttcagtgcagacacaaatgtggagttatcacttagaaagggagacagagtcgt

SEQ ID NO: 1146 - DNA, 146 bp, Gallus gallus
acagtaaagtctttattaaagttattgttgggtgatcacataactttctttttaaaaaaaaaaaacaactgctgtctgaattggaaaccagatcatacctttttgtattgggattttggaccatatatcttatgtttctgtacctc

SEQ ID NO: 1147 - DNA, 587 bp, Gallus gallus
acatgctgtttttctactgctgcaatctaacatgtgagttacagatccttaagatctttctggatgctccacaatgtgtctgcactcttcttcagctgagccacttcatcatccttcaacttttggttgatgacacttgtcaatccagaggcactcaggacacaaggcaggctcaggaagacatcgttctcaatgccatacatgccctttaccagtgttgacacagaatgaactctgcacaagttcttcagcatcgtctcacagagctcagcaacgctaaggccaatggcccagtttgtataacccttgagtctgattacctcataggcactttcaacaacctgcttgtggacttccttccagttctcactgtctttgtcagttcccatggcaggattcagctcctggagagaaacacctgccacattaactccgctccaaacagccacactagaatcaccatgttctcctaaaatccagccatcgcagctggttgggtggatatcaagtctctcagccatcaggtagcggaatctagctgtgtctagattgcagccacttccaatcacacggtgctttggcaggcc

SEQ ID NO: 1148 - DNA, 738 bp, Gallus gallus
aggtaccatgctgaggaaattcagtgcaatggaaggagttttcacaagacgtgcttcctctgcatggcttgcaggaaggctctggacagcaccacagtggcagctcacgaatctgaaatctactgcaaaacttgctacgggagaaaatacggccccaaaggtgttggctttggacaaggggccggatgtctcagcaccgacactggggaccatctgggcctaaacctgcaacagggatcaccaaagtctgctcgcccttctacaccaactaatccttcaaagtttgccaaaaagatcgttgatgtggataaatgtccccggtgtggcaaatcggtgtatgctgcagagaagataatgggaggaggaaaaccttggcataaaacatgcttccgctgtgctatctgtggaaagagtttagagtctacaaatgttacagacaaagatggagagctctactgtaaagtttgctacgcaaagaattttggtcccaaaggaattggttttggtggcctcactcaagtggaaaagaaagagtgtgagtgaaaaggagtggatgcaacagagtcaacctgctgctgatgtcagcagataatagttgtcaagtaaaaccaaatcaccacctactgctcataacctagggcattcattaaatattttccatcttgcaggaagccttctgaagccttctgaagaaaaagcaagttttcttagaatatagtgtttcagttttgttattgt

SEQ ID NO: 1149 - DNA, 489 bp, Gallus gallus
acctaaatggctttgttaattatggggatggccaggtgatgttattttttttaaagccgttcaggcagtcagtgtgtcagaagcagcacccaaacagtgagcgaacaaagtgctggccgcttgcactttgttcaaaaattgtcagtcaggtatggaaattacgacataatcagatccaatgtaaaacattaaagtaatacttacaggagggtaattgtaaatatcaccagtgccttacatttctgattcaacatagttatttgtgcatgtatgaaatactatgcacagtatcctctcttggtggtaggtaatcttcaacagggagcctctctacctgttgaggcattcttaaacatcaacaatgagttgaggcacaaaaattagttaaatgttgagcaggatagtcgtttgccaggaaactttctcctaccaactgttaattccaaaagttacatttcaaaatgtcataaaacaaatggtacctcggcc

SEQ ID NO: 1150 - DNA, 635 bp, Gallus gallus
caggtacaaaacctgttcaacactttgacctttggtcacatactcattgccaactttcactctagggtgtaataaacccttaactaaatcagaggagttgattcccattaggtaggcagctttgtcagcactttcagtgccatcagcctctgcctgctcttctctgggtcgctgtttgaatttcatgttcccaaagtgcataatggcacctgtgagcttgtaggcaccagacttctcatcttgcacaaatcccaaaatgtccatggcttgatctgttgccatcaattcttctccgtcatccaagttgtccactgtaactactccttgggagcaaaagtggtagtcatatgggttggtggagaccaatagcatatccagtaactctggcttctttcctgataagatctggtagaagatgtgatagtctctctcacccggttgctgaaaaatcactcgggatttctccagtaaatagatctcaatatcagcagatgacagcttgcccgtggttccaaaatggattcgaataaatttaccaaaacgtgaggagttgtcatttctcagggktttggcgtttccaaaagcttctagggctgggtttgcttgaatgatttgatcttccaaggttcccccag

SEQ ID NO: 1151 - DNA, 611 bp, Coturnix japonica
gggtaggtacctcgttgagtggataggagtatataaagggtgtaggaagcagttaagagcgttgcagttccggttaagataattgtgggaggggatcagttaaataaggcaattataattgttaattctgccattagattggttgttggagggagggctatgttggttaggttagctaggagtcatcatgtggctattaggggtaggaggggttgtaggcctcgtgtaagaatgagaatgcggctatgcgttcgttcgtagttcgtgttagctaagcagaataggagtgatgatgtaagtccgtgggagattattaggattattgcccctgaaaatgatcattgagtttggatcatgcttgcggcgattacgagtcctatgtggcttacggatgagtaggcaatgagagattttaagtctgtttggcgtaagcagatggagctggtcattagggcccctcatagggctagggtgaggaaagggaagtgtagatagttgcatacgggctgtatgagtagtgttactcgtataataccgtagccgcctagttttagtagtagggcagcaagtaatattgagcctgcggttggtgcttcgacatgggctttggg

SEQ ID NO: 1152 - DNA, 584 bp, Gallus gallus
gggcaggtaccagagcaaaattaagatgcagacagaagcacagcacgaaagagaactccagaagctggagcagagggtgtcactgcgcagggcacatctggaacaaaagattgaagaggagctcgccgccctccagaaggaacgcagcgagaggatcaagtttctgttggaaaggcaggagcgagagattgaaacgtttgacatggagagcttacgaatgggctttgggaatttggtcacattagattatcccaaggaggactatagatgagacgaaatttctttgccatttaacaaaaaccagacaaaatcaaacaaaatagttacaaaacttgcaaaaccaacattccccatgttaacgggcgtgctctctctctttctgtctctcttactgacatcgtgtcggactagtgcctgtatattcttactccatcaggggtcccctttccccctgtgtcaagtcccggtgcaggacagctcctggcggtcttttccatagtatgtcacagtattgatgtctctgtgcaatgattaaaaatgtttcagtgaaaaactttggagacgattttaatggagaaaaaaga

SEQ ID NO: 1153 - DNA, 591 bp, Gallus gallus
accgccgcctgcgatagggacggcgctgctgggcttggccttccgggatgttctctgctccttcgttcttctccccactctcactgttctggtagttttgctggtagttgcgtggaggacccctgcgacgcggatatcgtctgtaatggttacggtctgctgcgtatttgctgccttgcactggaacgccaccaggccctgtcacgttcgctgcctccgcacccttctctccttcaaccacatcaaactccacggtctctccgtctcctacgctgcggaggtacctcaaaaacccactcatttcagtcttagatattcacacatctcggtaaacaaaactaaaactacattattttttaatggccagcatgctgtattctctgagggctctaggcttgtgcagtagatacacataccacaaatgtaatgcttcctcaccgctgttaattgaaaatcttctaagttatttgctattgaggctgtggaaattgttaccacctgacaagactccaacagtggttaaatgactagtgtcggtagtgtaggaagctgtataaagataccttctgagctttccttaaattgtcacgt

SEQ ID NO: 1154 - DNA, 316 bp, Gallus gallus
actgtattttctgcttctctgccttttgaagccagggactgtcgggatttctttattctgtgggatactttacttctcagtctgaaaagctacttccttctacaaaggcaagaccaaagactttatgctggtccaatttgtagagcatagaggccccccccgactatttaagtttgacaatcttaatgaatttgtcatctttagagggaagcaaaagcataaaccataccaaagcaaaggaaatgctatatttttaaataagaaataataataatcacaggtcattaggatatcgtcagttccatggttctttagt

SEQ ID NO: 1155 - DNA, 523 bp, Gallus gallus
acagctggatgaggagctgggaggcagccctgtgcagaaacgagtagtgcaaggaaaagagccacctcatctgatgagcatgtttggtggaaagcctttgattgtttacaagggtggaacatctcgggaaggaggtcagaccacaccggcacaaacacggctcttccaggtccggtccagcacctcgggagctaccagagctgtagagctggatcctgctgccagtcagctgaactccaacgatgcttttgtcctgaaaactccctctgctgcttacctctgggttggccgtggctccaacagtgcagagctgtcaggagcacaagrgctgctgaaggttctgggagctcgtccagtacaaataggtcctatatacgttaagattctttggaaactgctaagtataaaggagtttgtaatccagactactatctttttggctactccaaaaaatctgcgggagtttccagcttatcatcagttgaaatctgttttgcaattgccaaataaatgcaagtaaaat

SEQ ID NO: 1156 - DNA, 642 bp, Gallus gallus
tgaaggttcttttacagcttctggggtgctcgggaaagacagatcttcagcactgccttcactgtctctttcataatcctttagcactgtcagcttatctacaccacatttctcatctaaaccagtttccagtttgccatctattttacttccaacatcctctctcaagctgctcagtccatgaccagcaccttttacttcccatattggctcatacggtttgaagtccacatactcttcccttcgaatagcatcttctttcaatgcctttttgtcatcatgatcaccatctctgcctttgtctttgctagaaaaggtatcttcctcatttttctcaaacaaatctttaggagtttctggagacatactcagaggctgtgcaggcattttctccagatcacataattctttagggccctctagctgcatttcttcttcagcttgaaataaaatggctgcttctgctttagtaagtgagggttccatttctgagtatttcatctcggaagcgtctctcttatttccctctgtaaggaatggatttcttacattttccattgtttctttacaaacctcacttgcactttctttgtaaatgcctcttgcagggaatccatctgagagtgaatcaaaggctgtgtg

SEQ ID NO: 1157 - DNA, 339 bp, Gallus gallus
actctgtctggattggaggctctatcctggcctctctgtccaccttccagcagatgtggatcagcaagcaggagtatgatgaatccggaccctccattgtccaccgcaaatgcttctaaaccggactgttaccaacacccacacccttgtgatgaaacaaaacccataaatgcgcataaaacaagacgagattggcatggctttatttgttttttcttttggcgcttgactcaggattaaaaaactggaatggtgaaggtgtcagcagcagtcttaaaatgaaacatgttggagcgaacgcccccaaagttctacaatgcatctgaggactttgattgt

SEQ ID NO: 1158 - DNA, 339 bp, Gallus gallus; identical to SEQ ID NO: 1157

SEQ ID NO: 1159 - DNA, 339 bp, Gallus gallus; identical to SEQ ID NO: 1157

SEQ ID NO: 1160 - DNA, 536 bp, Gallus gallus
acgcatgtgttatctacctcaaggtaacagcagtatgtggcaaaacattaaccacccatagtgcttctcattatgcacttctatttagccagcattattgtagtagctattcttattgaaaaccattcaatatttataaatgttctggtatgcattctttatagtgaagtgttaatatgcagcacttttatttattttagcaaataaataagtatatttctgtaattatagaaagtcaacttaatttttgagttacgtttcagataaaagtttttgtttagcactatggttttattgcctacatagctggatatatattaacatcggcttattctgaggctatccaatacattttttttctagttttcatttcaagtaaagcactcactgtgtataggaatttgtaattggaggtgcttgatctctacaaaagaaattaggaatcgctttattataaaatgctcctagaagtcttaattgtgttcatttctaaaaaattttgtaatgttagttgtgtgcatggaaataattaaggt

SEQ ID NO: 1161 - DNA, 363 bp, Gallus gallus
accatttcgctcagcaaggtccccgactccgcgcatccaatgccatgatgaataacaatgacctagtgaggaagagaagacttgcggagctgaacgggcctatttttcccaagtgcaggactggagtgtagctcccaggcagaggtcgttcccagcgggtctgtgctactgtgacaacctaaggcaaagaagtgccttgagagagttatttgtggtgcctcggttctgtttcatgcactaacagtttaaagtaactagtggctgtagttgaagatttttatccagtagcactgttgttttctgtagagctggaagctatccaagccagtaacctgccagtgttgtgcagcctcagctgagcgt

SEQ ID NO: 1162 - DNA, 428 bp, Gallus gallus
  misc_feature (403)..(403): n is a, c, g, or t
acctgctactttaaaacaaattttaactgcagctacttttcactaagcaagatggataaagcatgccatttatattttgccttctcaagagattattttcagaaacatatattattccaccgcaatctgacacttcctgtcatgctttcatcttgtaaaacctgaattccaattttaggctattccaggcttatgcttaaatgacagtgccttggtaagagaaaaaataattgtgctgcctttttctcccatagtgcctgaaaacatattgggcatacatatattatatatattcttacaaatgtccaggtcatgtataccagctgaaattcttttaatgtgggggtgtttgcattgtgagatttaatcaagacattaacatgagtagaaggttgttgttttnagacagaagtttgagaatcanctca

SEQ ID NO: 1163 - DNA, 472 bp, Gallus gallus
acacctctaccccgacaagcattacatttctgaacagctccagccttccctccttgaccattacatgcactacaaaggacattcttgctaagttgtagtttagttgtctttccattatataaatcttctaaagagactttgagaggatgcatcatatcttctcctcttcttctaccattacgacttctactctgaccacccatgaagttgaacaatccaccaccaaagatgtgggagaaaatatcgtccattccactgcttccaccactgccttctcgaaggccctgttctccatatctatcatataactcacgtttctctggatttgacaatacttcataggcaaagcttatttctttaaatttgtcacctgcatttggattcttatcaggatggtattccttggccagttttctataagccttcttgagctcgttgtcggaggctccgggcggcacgcccaggatatcgt

SEQ ID NO: 1164 - DNA, 554 bp, Meleagris gallopavo
acagatcatccagcttgcggaaggcgctttcagtccaaatgcagaaacgcccaacgtggccaccaggagcaagtctcagcaggttcagcttgttcacatcaagaagagtaatccccgggatattccggaaagctctaatgatgccgttgtcctcgttgtagatgatgcaaggtcccctgcgctggatgcgacggcgattcctcatcttacccttcccggccctcatgcgctgagaggcataaaccttttttatgtcattccaagctttaagcttcttaagaaggagaacagcttcctttgttttcttgtaactctcaactttgtcctcaacaaccagaggaagttctgggatctcctcaatgcggtgacctttagacatgaccagtgctggaagagctgatgctgccaaggcagaacagatggcgtaacgtttctgagttacgttcactctgcggtgccagcgtcgccaagtcttggttggggcaaacatgcggcctccacggcacatatttccaaaggcaccctggccagagcggtgagttccaccacctcgt

SEQ ID NO: 1165 - DNA, 554 bp, Meleagris gallopavo; identical to SEQ ID NO: 1164

SEQ ID NO: 1166 - DNA, 273 bp, Gallus gallus
caggctcacgctctgctgatccagaagctcttggcttaggctcctgatttagcactggcaagttttgtttgcatttctgtcacaattaaaaagtgttcctgaaccgcaatcgccaaagcaggggtgaattacaggatatagcacgacaaatgcatttttctgagagcaacacaacctatgcatgtgctgactagatacagcttcctagaaaaagaatagctttttcaaaataagagatacgattcttcacttctgatagagtaacttcttctt

SEQ ID NO: 1167 - DNA, 109 bp, Gallus gallus
  misc_feature (66)..(66): n is a, c, g, or t
ctgcttgttatgttggtgtctgcgatacggatatagaaagcctcttcatcccttggaawgcytccntttkcaatgccctgagctcttgagtggatcgttgccyagttct

SEQ ID NO: 1168 - DNA, 465 bp, Gallus gallus
accacctgagaggacrttrttrgcatamagatccttwckratrtcwatrtcrcacttcatgatgctgttgtargtrgtttcatgaatrccagcagattccatrccratraargatggctggaakarrgtytctgggcagcggaagcgttcatttccgatggtgatgacctggccatcaggaagctcatagcttttttccagagaggaagaagaggcagcagtggccatctcattttcaaagtccagggccacataacacagtttctccttgatatcacggacaattccacgctccgcagtggtaacaaaggagtagccacgttctgtcaggatcttcatgaggtagtctgtcaggtcacggccagccaggtccagacgcatgatggcgtgtggcaaagcatagccttcataaatgggcacgttgtgggtgacaccatccccagagtcaagcacaatccctgtggt

SEQ ID NO: 1169 - DNA, 465 bp, Gallus gallus; identical to SEQ ID NO: 1168

SEQ ID NO: 1170 - DNA, 465 bp, Gallus gallus; identical to SEQ ID NO: 1168

SEQ ID NO: 1171 - DNA, 275 bp, Gallus gallus
cgcacaaactgtgtagtgtcagacctgattatgggcaatgagtatttctttcgagtcttcagtgaaaatttgtgtggattgagtgaaactgctgcaactaccaaaaatcctgcctatatccaaaaaacaggcaccacttacaagccacctagttacaaagaacacgacttctctgaacctcctaaattcactcaccctttagtaaaccggtctgtgattgcaggatacaacgctacactcagctgtgcagtgagaggaatccctaagccaaagat

SEQ ID NO: 1172 - DNA, 342 bp, Meleagris gallopavo
acttctgggaatcaaaagtgaaatggaactaatcctagtttaattgcaattgctattgttagtagcagacatgatgtggggttgtttagttgtgtaatgtctcactgtcctgtagatcaggcattggtaaggcttgagaataggattaaggctgatgcagttgattgggtgaggaagtatttaatggcagcttcaattgctcgagggtggtgggattttgagatgagtgggataatagctaaggtgttgacttctaagcctgtccaggccaagattcaatggttgctggacatagtaatgctggttcctataattaagcttatggtggagattagttttgcg

SEQ ID NO: 1173 - DNA, 682 bp, Gallus gallus
aggtactccttatgattttgggacatgctcattgcacaagctctccagtttctattaaagctgaggatagttgccatctcttctttagtcagctcaaagtcaaacaccttgaagttctccacaatgcgctgtggtgtgacagacttggggatcacaatcacatttctctggatgtggaaccgaatgagaacctgtgctgctgttttgttgtgcttggctgcaatctctttaattttagggtcatccagaagtgagggatcctctggcttagcccatggtctgtcaggagagccaagggggctatatgctgtcacagagatccctttggattgacagtagttgatcagcttctcctgggtcaggtatggatggcattcaacctggttgtttgcaggtttgtatttcagtcctggcttgttcaagattctttctatctgctcatggttgaagttggaaatcccaacagcttttgccagaccagcatccaccagttcttccatggcctcccatgtttgtagaagatccgtgttgccaggtattgacatgcctttgtcatctgcaggaaataggtcctctcctgccttaaatccaacaggccagtggatgaggtagagatccagataatccagtttcgggctggcaagagtcttctggcaggctcctttcaccaatgatttttcat

SEQ ID NO: 1174 - DNA, 217 bp, Gallus gallus
  misc_feature (198)..(198): n is a, c, g, or t
aggtcggaataattcttcagcttcagggttgggctctttcagccattttgccctgttatagtccacaaatctaatcaacaaagggtggaggggatagaagtcccgtgccaaagtggtgaactcatctaggatgagcagttcagcctccgacatgtcacctctgccttctgctcttagatgctctgcatccgataccanccactgctgcttttttctt

SEQ ID NO: 1175 - DNA, 656 bp, Gallus gallus
acagattttggtttcacagaaatttaactgcagaaacaggaaaagcactccaagatatcagttgtaggtcatgtagcggggagtagcactgaattttgcataggatttaatgactttgttcactgcttctaagaagtctttctcagttgcaatttttcggcgtgctcggattgcaaacatgcctgcttctgtgcagacactgcgaatctcagctcctgtgctattaggacacagtcgagccaacagctcaaatcttatgtccctttccacactcatggagcgagcgtgtatcttgaatatgtgagtccgcccctcaagatcaggcaagctaaactctatcttcctatccaacctcccaggcctcatcagagctggatccagagtatcaggcctgtttgtagccatcaacactttgatattgcctcgtgggtcaaaaccatccaactggttgatcagctccagcatcgtgcgctgcacttcattgtcacccccagcaccatcatcaaagcgagcacctccaatggcatcaatttcatcaaagaatataagacaagcttttttagttctggccatttcaaagagttcgcgaaccattcgagctccctctcccacatacttctgcaccagctcagatccaatgactctgatgaagcagg

SEQ ID NO: 1176 - DNA, 345 bp, Meleagris gallopavo
cgccggcggtgcggctgcagacatggcgatccgctaccctatggccgtcggcctcaacaagggctacaaggtgacgaagaacgtatccaagcccaggcactgccgccgccgagggcgcctgaccaaacacaccaagtttgtgcgagacatgatcagggaggtctgtggcttcgcgccctacgaacgacgtgctatggaactgctgaaagtttccaaagataaacgtgctctgaagttcatcaagaaacgggttggcactcacattcgggccaagcgaaagcgggaagaactcagcaatgtcctggcagccatgaggaaagctgctgcaaagaaggattgagttgt

SEQ ID NO: 1177 - DNA, 305 bp, Gallus gallus
  misc_feature (261)..(261): n is a, c, g, or t
ccatgaaaacctgcacctattgacaccaaggggagaaagaaaaacacrgggcacttcagaatggattcagggaatttccactgaccttttaagaaatggcttgtggccaccttgatcctgagagattgtggttttaatttgaaagaattcatagattgaacacttgtaaaaattaataagcacctacgacaacgaagagtacacacgaggatttaaagggtagggatttttttttacgggtctgacttatcttcccggggnaaaatggttttataaaacttncanagaacttttttaagagccgt

SEQ ID NO: 1178 - DNA, 467 bp, Gallus gallus
  misc_feature (427)..(427): n is a, c, g, or t
aggtaccacagccaggagctgattcacatttcggatttgcaatctgaggtgcctcccattcgccatccatatcctcatcccaatcttcaggcttctcagcatcagggtctgctacgtattctggctcatcatccagccagccctctggtttcacagcattttcatctgctatctttgcaggagcatcttcatcccagtcatctggtttaacagcatctggatctgggattttgggtctctcatcccaatcctcaggcttctggtcatttgggtcctcaatctctcgaggtggattcacaggaggagacatatcatttagcaaattcccactgttgacaaccatttgatcaaccagaatttcaaaactattatcaggattcaaaaccagagtataaaggtgtgtcttcttatcagagaagtaggtctncaagtctgcatctggacgcttcrcgtgcttctcctcatat

SEQ ID NO: 1179 - DNA, 312 bp, Gallus gallus
gtcctgctgaaggctgggattcctcttgggactttgttgacttgtggatagcaggcgtgctctgctgattcattttcatcactgtcaaaatggtcatcaaagtctttgtagccacatcttcggggtctacagcgatcaaaaagaaacagcaagaggtagtgggctttttagaggccaacaagattgactttcagcaaatggacatagcaggtgatgaggacaacaggaaatggatgagagagaatgttcctggagaaaaaaagcctcaaaacggaattcctcttcctccacagatcttcaatgaggagcggt

SEQ ID NO: 1180 - DNA, 362 bp, Gallus gallus
acttagcagcaatcccaaagcgcgtgttgttactgccagcagtccatgcaagattcactgatgtctcaaccttattattaaccttctgataaatagacccaccaaactctgtgccatcattcacgtttgtgtgcagctgaaagtctcctgccttatatcccaaggcaaagttgttttgggaaagcttagatttggctgtatcaaaagccatctgatagccagcaagccagccttcataacccagcactgcccagccatagatggttggtccagagagatcaatgtctatattgcagcctaggtttacgtattctcttttgtaggaagtcttcaattttccactcttcttccctgtatttggt

SEQ ID NO: 1181 - DNA, 485 bp, Gallus gallus
accaagtggaataaaatacctttatcttcgaaacaatatgattgaggccattgaagagaacgcatttgacaatgtaacagatctgcagtggctgatcctagatcacaatcatctggaaaattcaaaaattaagggaagagtcttctctaaactaaagaatctgaagaaacttcacattaactacaacaatttgactgaagctgttggaccgctccccaaaactctggatgacctgcaattaagtcacaacaagatcacaaaagtcaatcctggtgcacttgaggggctggtaaatctgactgtcattcatctccagaacaaccagctgaaagcagattctatttctggggcatttaaaggtctgaattcacttttgtatctagacttaagcttcaatcaacttacaaagctaccaacagggctgcctcactccttactcatgctgtattttgacaataaccagatctccaatattcctgatgagt

SEQ ID NO: 1182 - DNA, 204 bp, Gallus gallus
  misc_feature (20)..(20): n is a, c, g, or t
ctgattccagcsgccccccnggcaggtactttctgatctgatggttattacatcaccsttgatgctgatagttaaattaggtttggscacaccagaccatcttcctgggaggaaaccccactcccagctctttcatatagtcctcaaagttttcacttgaaaggagcttccaggtgcccacaaactgggtcgcacattttgtca

SEQ ID NO: 1183 - DNA, 346 bp, Gallus gallus
accttggctttagttttattagcatgaaacacctttggcatgcttagcttccaggtaactggtaactccctacctgtatcagaattcattttacgtagttccacagaacatgaagatccttatttgctaagcctttgaaagctgacattctttttcataagagggtgtatttaaatggatgttcaccaatagacaataggccagcttactagtggtgagctaaaactgctaaatgaatgtccaacatgattgtaaggcttatgtcactcaagattttatcctttggattttcatgatcaaatttcatatactattgtatagacttctgctttgtagtgtacctgca

SEQ ID NO: 1184 - DNA, 331 bp, Gallus gallus
acaggttgaagatttttgcagcattagtgctcgtcactgccacaaactggttttcatccatctttcctgtagccactgctttgtcccagatgacagacatccgttcctcgatgccattggtcccctctgggatcgctgtgaagttgtcttttccaattgctttctgcgcagtgctgaacgtgcagtgggcacttcctgacacctgcaggccaccactggccaggagggagttaatgtagtcaggggttgtgggatctgggcttagggggggcgagaccacaaatgctgcagccttggcccagttcttgctccagtagtgtgttccatcagt

SEQ ID NO: 1185 - DNA, 525 bp, Gallus gallus
  misc_feature (518)..(518): n is a, c, g, or t
aggtacttattgcacagctaattggctattaacatcactgccatgctarcatccatccagcaggaggaagcagctgttgaaggcactgaaggaactaaacttgctctattaaaaagaggaaaaacctgttacttagacatccacatctgccattgctctctgcagaggatcaacagaccagtagtcgtcatcccagcccaggaaccagccagagaaggttggaggctcaagcccttgcttaacgagtgtgactggagttctcttatcacggctggctggatcagtttcaatgtatcgyttagcagatttcaaagcctcggtcttttcttcctcctgggcatcttttccaatccatacaaacacctgatcccatgtgtcaaggatcataacatcatctgtagcaaggtcatcctgggtcaggtctccagggacttcttcaatagtgaagcgtccactcttgttggagcatgcaaaaagacgaggggggtgagcatccatcttcttgtccttcagccgangagaagt

SEQ ID NO: 1186 - DNA, 224 bp, Gallus gallus
acttgttacaatacagcatggagaagttaccaagcgattggacacaacaaccttttctactttcttctcaaggatatctttcattattttgcaaaggttttcaaacttggcttttttctcttcctgtttctttttctcctcttcatcttctggaagctctaagccctcttttgttatagaaaccagagtcttgccttcaaattccttcagctgttgcacacagt

SEQ ID NO: 1187 - DNA, 224 bp, Gallus gallus; identical to SEQ ID NO: 1186

SEQ ID NO: 1188 - DNA, 427 bp, Gallus gallus
acagacataggtgtaactgcagttcactaacagcagcttaactccttggtgttgacagtggacattgtgctgggggcactcgaatcccagtgctgaaattaacactagtggaatctgtccttcatctttgcactgtggtatatctatgccatgttattaatcccgttctgtgcaatcagcagtgtgctaacctgctttttttcttctgtaagcatttcgcattattgggcttcattacctgccttgctttgtataccaaggctggttctcttgcacatcttacgcttttatacctttaactttttgaatggtcagatactgaactggacagtcaaacaacttgtgttctttagggagtcgtagctactgttgtattttaacactacagctgagggcttctttgagggcgggtttcttcttggagagt

SEQ ID NO: 1189 - DNA, 501 bp, Gallus gallus
actggttctgagagagcttttaaggtcccagagaagcaagctgctccaacctaagtcattacaacaaactactatgtcatatacttgtttgtaaaacccagtagagttttttgttgttgttgtgttatttttaaatatttgtttcttggtttaagcaaaatgacaagcggttatggtgattagatatagagtggggcaaattaagtgagttgatttagttgtgtgtataaataagtagtgtgtgaaagtgctcaactgcctaatggaatttaggacttttctaaatgtttatgcagacttagctattgcataactattggcctgataaccagagcggctgaggatgtggaacaaactacatatcagagttcactgggatgaatatatggtatctttggatggaagaagttcggtaaggattagttatttcagctccacataaattactttgaaggagttaggctgtcagaaagtgccaaatactcacttttgggctccagt

SEQ ID NO: 1190 - DNA, 312 bp, Gallus gallus
acatcaggagaatgagatgcttatttgtcacttccacataaagccaccaggatggtttcataatcccctttgagttcatccataattgcttggcgaagagatataccatacagactcttataataggctttgatctcattcaagtctacttcatggcgtgaaaccatgattctgataagctgtttgtgtcgtgtcccacttcccttcatggccaagtggagtttttcagcaaagaaagctggcttgcttgtggcacacttcacaagggcagtcaagcagttttcaatatcacctttcagctccaaatcaagt

SEQ ID NO: 1191 - DNA, 592 bp, Gallus gallus
acaagttcttcaagggaaagaaccgccatgcttcctgcagtgcttccaaggagggatgattgtgcatgctggaagaagggaagaggaagaagaaaacgcacaaagtgactggagactatattgtgtgcgaggagaagttcccaatgaaggaaacttacttgaagtagcatgtcactgcagcagcctgcgttcccggacatccatgattgtcctcaatataaataaagctcttatctatctgtggcatggctgcaaagcacaatctcacaccaaggatgtaggaagaacagcagccaataaaataaaagaacaatgtccgctggaagcagggctgcacagcagcagcaaggtgacaatacatgaatgtgatgaagggtcagagcctttgggattctgggatgcattaggaaggcgagatcgaaaggcctatgattgcatgttgcaagatccaggaaagtttaatttcaccccccgcctgttcagcctcagtagttcttcaggagaattctcagccactgagttcgtttacccttcaagagaccctgctgtcatcaattctatgcccttcttgcaagaggatctttacactgcc

SEQ ID NO: 1192 - DNA, 260 bp, Meleagris gallopavo
accatcgaaagttgatagggcagacattcgaatgggtcgtcgccgccacgggggcgtgcgatcggctcgaggttatctagagtcaccaaagccgccgggcgagcccgggttggttttggtctgataaatgcacgcgtccccggaggtcggcgctcgtcggcatgtattagctctagaattaccacagttatccaaggaacgggaggggagcgaccaaaggaaccataactgatttaatgagccattcgcagtttcactgt

SEQ ID NO: 1193 - DNA, 305 bp, Gallus gallus
gactctgtccgctgtgggttcggtgccgccatggccaagtccaagaaccacaccacgcacaatcagtcccgtaagtggcacagaaatggcatcaagaagcccagatcccatagatatgagtccctcaagggggttgatcccaagtttctgagaaacatgagatttgccaagaaacacaacaagaaggggctgaagaagatgcaggccaacaatgccaagcaggcagctctacagaaaaaggactgacctggtttaagacaaagaaccagtttgccttttggcatgtgtgtttaaagcatttttgt

SEQ ID NO: 1194 - DNA, 361 bp, Gallus gallus
caggtactgaaaaacttctaggcttccagcttcaccgactctagaatggaacgtgcttctttatactcttcagcttcttccaactctttttcattttctagtagttgagtgagatctgcatgtgcaatttctaatctccgctgacagtctggaatcatcattcgagactcttgtaagatctcaacctgcttttttattccatagtcatcacatgcttcagctttcattttttcaatcctctcttcttgttgttttgcttcwttttcrtacataactttttcttttgccaatcgcttcacgacgccggttttgatcttgatctgcctcagacggggatcggmcatggcggggchgcagcgcg

SEQ ID NO: 1195 - DNA, 271 bp, Gallus gallus
ccgggcaggtacgttcttgaagggttaatggtatgtgatttatactgtgccttaattgttatgctatttaaaaacaaatatttattttgaaagttttactatgctgtgctctaaagaaagcaactttagatgtgacactgtataattatgtattcatctcatggcataaattatttagtagacttagatgtmgcatattaaatatkaacctaattaactaaggatgttgacttggatttatttaaattcwgtatgtgcactgtatgagggt

SEQ ID NO: 1196 - DNA, 270 bp, Gallus gallus
ctgcagctccagcagcgcccggtccatcttgttcatcatcaggacaggtttgatcctctcagcaatggcctgacgcagcacggtttctgtctgcacacacacaccagagacgcagtcgacaacaaccagggcaccgtcagtgacccgcagagcagcagtgacctctgaagagaaatccacgtgcccaggagagtcgatcaggttgatcaagaaaccagaaccatctttgctctgcttgatgaacgccagatcgttttcagagagctcgta

SEQ ID NO: 1197 - DNA, 515 bp, Gallus gallus
  misc_feature (428)..(428): n is a, c, g, or t
aggtacaggctagcatcttgcagaggaagagcttacttcctctggtctagtttccttacacttaaaatgaaaggcaatacagaatcttattctacttctgccttgagaaaaacaaaataatttactttccttatatagcttagtgctctgaaaacttagttcttaagttaaaccagaattattttacacgaaccttttcatcagatgcaatcttaccacttgtcagactcttccccagtatacattacaaagctgcttagtaagaaaagttgtgtgaaagcagcttctaattaatggatcacatgagatcctgcatcatccccagtagcagcagtctgctagcaaccrcagaaatacattagcaaaggttacaccgaagcagtcatgtctgacagctaatacagcactataacatacagacctttcrnangcaggtcagtatgtagaaataattctttancatgtaaacaggaaaactgatctgtcagttacrtagatcaacagctgaagctatt

SEQ ID NO: 1198 - DNA, 160 bp, Gallus gallus
  misc_feature (113)..(113): n is a, c, g, or t
aggtaccgcctgcagagggagaaggaattcaaagccaaggaagcagcggcgcttggatctcatggcagctgtgtacaagcttttttktttkttttttttttttttttkttggnttttttttttttttttttttccacaaaaaaaaaactttcttatgkkt

SEQ ID NO: 1199 - DNA, 1252 bp, Meleagris gallopavo
ctttctgttgacgagctttcatcttggaggaacgggttctgtgaagcattcttcagagtgaagtggtcctaattcttcctggaaccattgcaacccattccactcagggagccaatcctatcaattcttctgccgaagcagccagaatctctcatcatccggggcatctgcacccccctcagtctcttgaggaaggggttcctgtaggacagaggagtgttggatgctagcttgggttcagccttctgctcatcgctgtcatctatgagttctggtggaatctcctctcgggtttggggctcttgtaggtcaggattggactccagggcttcaatcagtgcgaatttgtcctccagtctctccagaagagcctccatgctggccagttctttggcagggctgaggttgtagatggggttggccctgctgggctgcagctggacaagaagcagcaagaggaaaccacaggaaaatgagcctctagtgtccatggcgctgggttcgttgggaatatgggaagttcaagctgtttcttctgagatggctcttcaggtctctctcttacttggacgaaggccggttcttcgaaagtgtggtatgggggtggacatccgtggattcattcaatgttggtagaggttagtwcaggrygtmgtccactctaacaaacctattgaccataactctatcctacataatcccaatcctaatcgccgtggccttcttaacacttgtagaacgaaaaatcctcagctacatacaggcccgaaagggcccaaacattgtgggcccttttggtctacttcaacccattgcagacggagtaaaactctttatcaaagagcccatccgcccatctacctcctcccctttcctcttcatcataacacccatcctagccctacttttagccctcacaatttgaacacccctcccactccctttcccccttgcagacttaaatctaggactactatttttattagcaatatcaagcctaactgtctactccttactttgatctgggtgagcctctaactccaaatatgctctaattggggccctccgagccgttgcccaaacaatctcatatgaagtcaccttagccatcatcttactagccacaattatactgagcgggaattacacrctaagtacaacacaaaaagcaagcaagctggagggcctgttgatgtaggtcccgagtttcagaaagacatgaatgaatcacttgccaggcttcagcggatgt

SEQ ID NO: 1200 - DNA, 544 bp, Gallus gallus
actacattcacaaagtcttccgatacgtccttcattacatctgcatgctccacattcaaatgtcccatttccgtcgtggcaggcggggctgtttggttctccttcgctttgacacagacaatcacggatgaactggagattgatttccacttcttcagtgaaccccagtggtttaattttaatggtttcattttgtcctttctttggacattcatttgctgttacattaatctcaaacctaacctcatctcctattgaaatgttggaacattttcttccgtcttcctgcgtgtcgttgactccattcttgcagtatgatttgtaactgattgtcactccttttggtagcttactgttttccaggatcacctctgaagaaagggaattgtatgcatcaatgatcaactgaatgacattgctggaattggaagacaacgttcctactgctgattttggtatgaggtttttcagttccttataaactgcctgaaactcttcagtaacagcaaaaattgtctgaatattgttctcactaagcttctgt

SEQ ID NO: 1201 - DNA, 624 bp, Gallus gallus
  misc_feature (621)..(621): n is a, c, g, or t
actgtataaaaacttgtgttgagttggaggtataaaagcccagttgtctgtatcaataatcaatgatgtttttgggaattttagaatagctgctgagaaattcacccacttactgataagaggcaacagctgctgctcatcgctttgatcacagattttgtaaggctttttttttccagcaactgtttgggcctacagcttctctatcaatattgcagaagcacctcctcctccattgcaaattcctgcaagaccatactgcccttgtttcagtgcatggaccatgtgaacgacaattctcgctccagacattcctatcggatgcccgagagagacagcgccgccattgatatttactttttgtggatcgatacccagcattttaatattggccagcaccacaacactgaaggcttcattgatttcccacattgcgatgtcttcttttttcagacctgtctcacttagaatcttgggaacagcgtgtgcaggtgcaatgggaaagtcaataggatcaacagcggcatctgcaaaagcaactacccgtgccagtggtttaactttcagtctcttggctgcctctgtagtcatcagaaccaaagcagctgctccatcattcanagt

SEQ ID NO: 1202 - DNA, 372 bp, Gallus gallus
aggtgaacgcattcaaggtgtttgatccagagggcaaagggctgaaatctgcctacatcaaagaaatgctgatgacacagggcgagaggttttcccaggaagagatcgatcagatgtttgctgccttccctccagatgtctctggcaacctcgactacaaaaacctcgtccacgtcatcacacatggagaggagaaggactaatccatggattcagcactggggttagcactgtgggatcacctccatgtgggtcacactgcaggttccctttgtccctctccctggagctgcagagctgttcttcatggggataacaacccagaacagcagccacatacaataaagtgcattttggtgagagtaaaaaaaa

SEQ ID NO: 1203 - DNA, 618 bp, Gallus domesticus
aggtactagaaacacatgctatgtatgtcatttagaaatgtagtgctgcttctagatgagacaactcttgaaggtgaagtatagtttcacgtagctctacgtcccttcccagagagtaaaacaattcccttcacccttaacttcccatttactttatccaaaatcaggaggaaccaacaacgcaccatagattctctacagtccacccttgattctgaagcccggagcagaaatgaggctatccgtctgaagaagaagatggaaggagacctcaacgagatggaaatccagctcagccatgctaacagacatgctgcagaagcaaccaagtcagcacgtggcctgcagacacaaattaaggagctccaggtgcagctggatgacttgggacacctgaatgaagacttgaaggagcagctggcagtctctgacaggaggaacaaccttctccagtcagagctggatgagctgagggctttgctggaccagactgaacgggcgaggaagctggcagagcatgagctgctggaagccactgaacgtgtgaacctgctgcacactcaggttggcttttcctgggttaaactgagcttcacctgttaagcactgacactggga

SEQ ID NO: 1204 - DNA, 581 bp, Gallus gallus
tgcaatggaaggagttttcacaagacgtgcttcctctgcatggcttgcaggaaggctctggacagcaccacagtggcagctcacgaatctgaaatctactgcaaaacttgctacgggagaaaatacggccccaaaggtgttggctttggacaaggggccggatgtctcagcaccgacactggggaccatctgggcctaaacctgcracagggatcaccaaagtctgctcgcccttctacaccaactaatccttcaaagtttgccaaaaagatcgttgatgtggataaatgtccccggtgtggcaaatcggtgtatgctgcagagaagataatgggaggaggaaaaccttggcataaaacatgcttccgctgtgctatctgtggaaagagtttagagtctacaaatgttacagacaaagatggagagctctactgtaaagtttgctacgcaaagaattttggtcccaaaggaattggttttggtggcctcactcaagtggaaaagaaagaatgaagccttctgaagccttctgaagaaaaagcaagttttcttagaatatagtgtttcagttttgttattgt

SEQ ID NO: 1205 - DNA, 1242 bp, Gallus gallus
cgctggggccgttgacgtgcagcaggaacactataaaggcgagatggtgaaagtcggagtcaacggatttggccgtattggccgcctggtcaccagggctgccgtcctctctggcaaagtccaagtggtggccatcaatgatcccttcatcgacctgaactacatggtttacatgttcaaatatgattccacacatggacacytcaagggcactgtcaaggctgagaatgggaaacttgtgattaatgggcatgccatcactatcttccaggagcgtgaccccagcaacatcaagtgggcagatgcaggtgctgagtatgttgtggagtccactggtgtctttactaccatggagaaggctggggctcatctgaagggtggtgctaagcgtgttatcatctcagctccctcagctgatgctcccatgtttgtgatgggtgtcaaccatgagaaatatgacaaatccctgaaaattgtcagcaatgcctcgtgcaccaccaactgcctggcacccttggccaaggtcatccatgacaactttggcattgtggagggtcttatgaccactgtccatgccatcacagccacgcagaagacagtggatggcccctctgggaagctgtggagggatggcagaggtgctgcccagaacatcatcccagcatccactggggctgctaaggctgtagggaaagtcatccctgagctcaatgggaagcttactggaatggctttccgtgtgccaacccccaatgtctctgttgttgacctgacctgccgtctggagaaaccagccaaatatgatgacatcaagagggtagtgaaggctgctgctgatgggcccctgaagggcatcctaggatacacagaggaccaggttgtctcctgtgacttcaatggtgacagccattcctccacctttgatgcgggtgctggcattgcactgaatgaccattttgtcaagcttgtttcctggtatgacaatgagtttggatacagcaaccgtgttgtggacttgatggtccacatggcatccaaggagtgagccaggcacacagcccccctgctgcctagggaagcaggaccctttgttggagccccttgctcttcaccaccgctcagttctgcatcctgcagtgagaggccagttctgttcccttctgtctcccccactcctccaatttcttcctcagcctgggggaggtgggagaggctgatagaaactgatctgtttgtgtacct

SEQ ID NO: 1206 - DNA, 573 bp, Gallus gallus
acaacattactaccagctttttgatgcagacaggactcagttaggagcaatatatattgatgcatcatgccttacgtgggaaggacagcagttccagggcaaagcagctatcgttgaaaaactctctagccttcctttccaaaaaatacaacacagcatcacagcacaagaccaccaacctacacctgacagctgtatactcagtatggtagtgggacagcttaaggctgatgaagatcctatcaygggattccaccagatatttctattaaagaacatcaacratgcctgggtttgcaccaatgacatgttcaggctagcattgcacaactttggctgagctggcgaccccgaggcacctgttctttttttcttcttctctcctcttactgatattattcacactcacagaacattccaaatatcatacacaaacctgcagcactgcagagcgtgagcaagcaagagctgtgacctgcccttctgctgagtttacattgtcactagatgagttccttgtgcatgatgtttggaagttagttagctgcatttgacaagagaaatttgtgttgt

SEQ ID NO: 1207 - DNA, 411 bp, Gallus gallus
aggtatgatcctccaatggaagctgctggcttcactgcacaggttattatcctgaatcaccctggccaaatcagtgctggttatgcccccgtgctggattgccacactgctcacattgcctgcaagtttgctgagctcaaagagaagattgatcgtcgttctggcaagaagctggaggatggccctaagttcctgaaatctggagatgctgccattgttgatatgattcctggcaaacccatgtgtgttgagagcttctctgattatcctcctctgggtcgttttgctgtgcgtgacatgaggcagacggttgctgttggtgtcatcaaggccgtcgacaagaaggctggtggagctggcaaggtcacaaagtctgctcagaaggcccagaaggctaaatgaaaattctgt

SEQ ID NO: 1208 - DNA, 411 bp, Gallus gallus; identical to SEQ ID NO: 1207

SEQ ID NO: 1209 - DNA, 259 bp, Meleagris gallopavo
actgggagaagctctccacacacatcggcttgccaggaatcatctccacgatggccgcatcgcctgatttcagggatttggggttgtcctccagcttcttgccggagcgccggtcgatcttctccttcagctcagcgaacttgcagacgatgtgtgcggtgtggcagtcgatgacaggtgagtatccagcactgatctgcccggggtggttcaggatgatcacctgagatgtgaactgtgctgcctcctgcggcggatc

SEQ ID NO: 1210 - DNA, 625 bp, Gallus gallus
gagatgaagatcacatatgcacaatgtggagatgtcttgagggctttggggcagaatccaacccaggctgaggtcatgaaggtccttggcagacccaaacaagaagacatgaactccaagatgattgactttgagaccttcctgcccatgctccagcatatcgccaagacaaaagacacrggcacctatgaagactttgtggagggtctrcgtgtgtttgacaaggaaggaaatggaacagtgatgggggctgaactccgccacgttttggctacactgggtgaaaggttgactgaagaggaagttgataarctaatggctggccaggaagatgccaatggttgtatcaactatgaagcttttgtgaaacrtatcatggctaactgaacaccaggacaagacaggcgtggagaagcccggattctggccttggattttgatttattggaatgtcctctcatttttcagtccagattcctacttcaaagctataaaatgtattgtccctgaagttatttggataaatgcttgtttgttttgtcttgtttcctcatgggaagaaaaaaggaaattgaacaaacagaaccagaaccatgaataccttattgcattgtatgcaataagg

SEQ ID NO: 1211 - DNA, 453 bp, Gallus gallus
gaggatggcagcggcactgtggactttgatgagttccttgttatgatggtccggtgtatgaaagatgatagcaaagggaaaactgaagaggaactatcagatcttttcaggatgtttgataagaatgctgatggctacattgatcttgaggaactgaagatcatgctgcaggcaactggagagacgatcactgaggatgacatagaagaactgatgaaagatggggacaaaaacaatgatggcaggattgactatgacgagttcctggagttcatgaagggagttgaataaatctgaggccagatggacagcccgaatctctgaaactccttctgctctctgactcagctccttggttccatcccctggctgccagcatgaagactgagcactgagaagggtggccgtagggaaaataaagcacattgctgtcaaaaaaaaaaaaaaaaaaaa

SEQ ID NO: 1212 - DNA, 644 bp, Gallus gallus
acctgattcttcttaacaaatggaggaaatgatgccccatcagtgccgttaaccaaatcgcagtaaccttcccagtaagacagattccttttgtttttataactttcaattattgctgttttgcttatgtcttctttcccagtatacactctgtaaagtccatcagatgtcccattatacgggtagaagactcccagaactgggtccaaggggaagggaaccttgcttaagaaggggtctttgtatccccatagtatttctttcactgttctgttctgcagcatgtttgatttagaagatttaatccaagtatttaaaagtaggaggatgaaattgtttgtatacagggcaggtgcagcaacaacagcgaggttgaggcacgtgatggtgtcattttctgtcccaacagacatatcaggttcaaaacgagcagcgttaggcaacatgtaagatattgtgccattagagttttctgtaatattttctttaggtaaatatcgcaccctatatgtgtaaggtcctctttgttcaagttttggacgtgctccatagttcaaaacctctgatggattttccacattaaagatccaaaattgcctgtaaacagagcttcctggcacaagccaattatcatatgcaatggt

SEQ ID NO: 1213 - DNA, 268 bp, Gallus gallus
actgccaccaaacccagaaccaaggccagcatcttccatgactcaaagcttgttttcaagaaaaggcagtgagctttggagcggagagaaggtaaaaagcagcagctatcttgtagaacttcatcatgagattggcaatggacatcctcttgttactgcacaaccttctatcaccagcacattttattctgaaccaacctctcaccctacaattgctgaatgagagtagaaacacaggtttgcagattattctgtcaactgcagaagt

SEQ ID NO: 1214 - DNA, 208 bp, Gallus gallus
acttatcttcaaaaacagccatagctgccagtgagccagaacccatggtaacataaggcaacttgtcagttgatccatggggatatatgctgtaaaggtgaggtccagtgacatctacacctcctaaaaccaaggcagcaccaatgtagccttgatacctgaaaagcatttgctttagcattcgattagctgtgaccacacgtggaag

SEQ ID NO: 1215 - DNA, 395 bp, Gallus gallus
acccttcctattaaagatcctcacgtagacagtgcatctccagtgtatcaggctgttctcaaaactcaaaacaagcctgaagatgaaactgaagattggagccgccgttctgccaacctgcagtctaagtcttttcgcatccttgcccagatgactggaacggagttcatgcaagatccagatgaagaagccctgaggagatcaagggaaaggtttgaaacggaacgtaacagcccacgctttgccaaattgcgcaactggcatcacggcctgtcggcgcaaatccttaatgttaagagttaaaagcccacgttcagtgggcaaagatgtgagagagaattacaggaaagaaataactgctatcctgagttagagcctaacaacgtaacacacgt

SEQ ID NO: 1216 - DNA, 287 bp, Gallus gallus
acattgaaggtctcaaacattatctgggtcatcttttcacgattggctttagggttcaagggtgcttctgtgagcaaggtggggtgctcctcaggggccacacggagttcattgtagaaagtgtggtgccagatcttttccatatcatcccagttggtgatgatgccatgttcaatgggatatttcaaagtaaggatacctcttttgctctgtgcttcatcacccacataggaatctttttgacccataccaaccataacaccctggtgcctggggcggccaacgat

SEQ ID NO: 1217 - DNA, 114 bp, Gallus gallus
atattcattccaagaacttcatccaccgggatgtgaagccagacaacttccttatggggcttggtaaaaaaggcaacctagtatacatcattgattttggtttggccaagaagt

SEQ ID NO: 1218 - DNA, 582 bp, Gallus gallus
acacaatgcagggtgcacgagcctgtgcttctctgaagaggctccggactcgtgcggctccaagacctcctatcacctccacaaattcagagcctgccatggccaagaaaggcacctgtgcttctgtggccactgcctttgccaacaacgtcttcccgcagcctggtggtccaagcaacaaggcacccttgggcactttagcaccgagctgaaggtagcgatcaggattctttaggtagtccacaaattctttgacttccatttttgcctcgtgcattcctgctacgtccttgaagccaattcctttcccggattttccgtccacaatggtgaaacgagccattttcagctgattaaaagcattgaatcctcctgcccggcccgcaaccctgataaggcggaagatgctccacaacatggacagagccaccagtgtcactatcagggaaatgacatcatttccgtaaaagccggggtgtttgtaggaaacagggattctctctctctcatcaatattcagctcgtcctccgcagctctcagcttctcttcgaacttgtcgatgtttgccactcgcatggtgt

SEQ ID NO: 1219 - DNA, 329 bp, Gallus gallus
cagctttggaaaacactatctttaacattaaggtgtaaaggatgaacaacacaaaattaaagtgtgtgctgtattgctagaatgcatcccttctctctgttctccacaaggatatgttcccattaacagtctagtctatgaaacaaatgtttttcccaatgaaaacttgaaattgttccattgtggaccaattcttaagagagcagtagcaggagatgcctctgaatctgcacttctgaaatgcattgaattgtgctgtggttctgtcaaagaaatgagagaaagatatcccaaagtggtggaaataccgtttaactctaccaataagt

SEQ ID NO: 1220 - DNA, 661 bp, Gallus gallus
acgggtcaagcaaagaagtcacagttaggggccataactgtccaaaaccaataataaacttctatgaagctaactttcctgcaaatgttatggaagtgattcagaggcagaacttcaccgagcctactgcaattcaggcacaaggatggcctgttgccttgagtggattggacatggttggagttgcacagactggatcagggaaaacactgtcttacttgttgcctgctattgtgcatataaatcatcagccattcctggaaagaggagatggacctatttgtcttgtgctggcaccaactcgtgaactggctcagcaagtgcagcaggtagctgctgaatatagcagagcatgtcgcttgaagtctacatgtatttatggaggtgctccaaagggaccacaaattcgtgatttagaaagaggtgtggaaatttgcattgcaacacctggaagacttatagacttcttagaagctggaaagaccaatctcaggaggtgcacttaccttgtccttgatgaagctgacaggatgcttgacatgggatttgaacctcaaatcagaaaaattgtggatcagataagacctgacaggcagactctgatgtggagtaccacatggccgaaggaagttaggcagctggctgaagactttttaaaaga

SEQ ID NO: 1221 - DNA, 343 bp, Gallus gallus
acttgagcacgacaagtttaaccttcttcctcttatgcttgttcttcttgggggtggtgtaagacttcttctttcttttcttagcaccaccacgcagtctcagcacaaggtgaagagttgattctttctggatgttgtagtcagacagcgtgcggccatcttccagctgcttcccagcaaaaatcagtcgctgctgatcaggaggaattccttccttatcctggatcttagctttcacattttctatagtatcagagggctcgacctcgagggtgatggtcttccccgtgagggtcttcacgaagatctgcatgtcgaggcccgcacccgcggggaagaggcg

SEQ ID NO: 1222 - DNA, 343 bp, Gallus gallus; identical to SEQ ID NO: 1221

SEQ ID NO: 1223 - DNA, 383 bp, Gallus gallus
ccgggcaggtaccttttaaccccatggaaaaaatatctaacgttcattactaccaataacaggaagaagattttgcttcgagaatgacaaacccatcatggtgaagtttaggcacgctccccacgaatgcggcgtgctagctggatatcttttggcatgattgtgacacgtttggcatggatagcacacaggttggtatcttcaaacaggccaaccaagtaggcttcacttgcctcctgcaaagcaccgatagcagcgctctggaagcgcagatctgttttgaagtcctgagcaatttcacgcaccagacgttggaagggaagtttgcggatcaaaagttcggtagacttctgatagcgcctgatttcacggagggccacagt

SEQ ID NO: 1224 - DNA, 473 bp, Gallus gallus
acatgaggactccaactgctcctgcctctttggcatttgcaaccttctcagcaagtgttatttttccagctctgacaatgactatggttccattcaatggagtcactgacttctgtattgtctcaatatcttttttcagtccatagttcacatagacaggtttgccagaaaaagagccactctcactgtaggccacgtatgcatcaggcatctccaagatctcctcgctatcattgatcaaaacggacactttgttcttggtgctgcctctgatttgcaacttaatatagtgttcatcgttccacactttatccaagaagaaactgttgaattgctcatgaatgtaggtggccatgtttgtatcttcagcctcaccagcctcaaaggagtccaaacctgccctttgcctcaagcggtctccaaggttcttggctaacagcttatctgacaacatggcttttaattgaggccag

SEQ ID NO: 1225 - DNA, 185 bp, Gallus gallus
gtttgttgctggaacacatcaattgtatcttcatcctccatttccaactgkgcgggggtgtctgtttcattaattggctgcccatcgaaccggaatctgatttgcctcatcgacaacccctgtcgttcacaataggctttcattagtttactaagkggggtatgcctcttaatcttaaactgcac

SEQ ID NO: 1226 - DNA, 337 bp, Gallus gallus
accctcgggcagcttaggcagtctcaccgtttctgcatcgagcaaaagcacaccatcactgctgtgcagtttgatgtccatctgggaaagagcttcaatattccctgcttgcgctttgatgttaatgcctcttggagcatccatgctcagagaccgagttggtgactccagccttagctgtttaaaagcttctgccttcacgagaggtgtttctacagagtgttcaaaaagtgctccttcaggccctgtgactcgaagtttatctgttccaatgacaacctcattttcatccactgtaaaaagtggcttgccatcctttgagttgatctgaaattgc

SEQ ID NO: 1227 - DNA, 606 bp, Gallus gallus
actccttcatgacctgaataaagtatggcatggcaaagtccatgatgttgtgcctccacgctgtttccaagacaacgtcaggccttaaaagatcatagcaggtgaagagacaagcaccgaagcactctttcttgttctcctgcaagaaccactgcaacaattcttctgccaactcagtatctttggattctgaagcatattgcattgcatccttatacagtctgtccttcttacagagttccacactttgcttccagcggttgtttcctttgaacagatatgcagcaatccttcggaactctatcagctcgtgtttctccaaacgttgagcaagagaaatgttgtcaaagttgtcataagcatctatagaagtcctaagggcctggtagtcttcttcaataatgaagaggttgttcagggactcattcactgatttgttgttgtgattttgaacagagcgcaagtaaggtttaaccaggggtaactgtttaaccttggtgaagaaggtaacagcacgagtatggtcaagtcgaggggacaataccatcagcagatcattgagcaacagaggtttgaattccaaatagaattgaactgctttgtagt

SEQ ID NO: 1228 - DNA, 363 bp, Gallus gallus
actgagcctgctcaggaggcagctctcgacgtaactcatctgccaggatatacggcttatctgaagccaggattctgaaggaagctatcacttgctcagccgtgtccgtatctgctgtttctctggtcatgaagtcaatgaaggactggaaagtgacggttccttgtccattggggtcaaccagagacatgattctagcaaactcagcttcgcccaaatcataacccattgaaatcaagcaagctctgaaatcatcatggtccatcagtccattcttcctcctgtcaaagtgattaaatgatgctctgaagtcattcatttgctcctgggtaatacccttggcatctcttgtgaggatctgag

SEQ ID NO: 1229 - DNA, 441 bp, Gallus gallus
actgtgatgagactgccttactcaacaccttcttttgaaagaggaggtgttaaataaagattaaggtttctgagtcattacagttcttgtaaatgcactcacttattcttatgaatggctgagagacttatactttatccactggttggggccacgcacttcaaagggcggctcattgaagtaaatgtggttaccgagttcttccagtgggggctctgggtcagtggtagcaaactgagcagcttcctctatctcttttctcactgccacatcgatttcctttaattcttcaacgctagcaaggttattgttgatcattctgtccttcagcaaagtaatgggatcacttttgcttctcacttcttgaatttcctctctagtactttttttctcatcctgtgcttatgcccaggaaggaatttctgttatctattattttgt

SEQ ID NO: 1230 - DNA, 219 bp, Gallus gallus
acattcatgacaggtttcttttttctttctaaagaaaaaggcctttctgttttgttagcactttggtgtttcaatgtgataatatttaaaaactggatttaaataagaggttagtcaggaaaaacacacaacaatgcttgcaaagtgctccacaccgctgtagccacaggagtgtgaacacactctagaacacgggggcatccagccacggtgctctgt

SEQ ID NO: 1231 - DNA, 575 bp, Gallus gallus
gtatcattgtatttttcttctgcaattttcttgatcatatcaagagttttacgaacaagcttctttctgatcacctttaataacttatgctgctgaagtgtttcacgagatacattcaaaggaagatcatcagaatccacaacacccttaacaaagttaagatatttgggcatcatgtcatggaagtcatcagtgatgaacactcttctaacatacagcttaatgaaatcactttttttggatccatactcatcaaacaagccacgtggagcagaattaggaacaaacaagattgatttgaaagttacttccccttcagcagtaaaatggatgtaagccattggatcatcatgttccttggaaaatgttttgtaaaaagctttatattcatcctcttcaacttctttagatggtctctgccagattggttttatgtcattcatgagctcccaatcccagacagtcttctcaaccttcttagtttttggtttcttctcttcctcctcttcttcaactgcagcttcatcatcatctgtttcttctttttcctcctttgctccctcctcttcaatggg

SEQ ID NO: 1232 - DNA, 619 bp, Gallus gallus
  misc_feature (598)..(598): n is a, c, g, or t
accagtaagcatatgaacttcaaaatgcacaattgccacagacagttgacttgaatacagtaatggtggttggttgcacacttagagacgacttttagattcttccactctcaaatggctttgcatttctggatcatctagtcatgcactggagaggaattccacagctgtctccttctcttcagttaactccttagcagtcagatccatcttctcacgagaaaagtcattaataggaagaccctcaacaaacttccaggtcttgtccttgatcacaacagggaatgaatatagcaagtcttcaggaacaccataagaattgccatcagaaatgactcccatggaaacaaattctcccgctggagtgccaaaccagatgtctctcacatgatcacagattgctttggcagctgacattgcactggacagcttcctagccttaataacagctgctccacgttgctgaacagtcaggataaagtctcccttcagccagctgtcatcttttatagcttcataaactccaacttcctttcctttcacattcacctttgcatggttaacatctggatattgagtggaggagtggttgccccanatgatgacattcttcacatcg

SEQ ID NO: 1233 - DNA, 426 bp, Gallus gallus
acgttgacaaccatattggtatctcaattgccggacttacagctgatgcaagactcttgtgcaattttatgcgtcaggagtgtctggattctagatttgtgtttgatagacctcttccagtgtctcgtttggtgtcactaatcggaagcaaaacgcagataccaacacagcgctatggcagaagaccatatggtgtaggactgctcattgcaggttatgatgatatgggccctcacatcttccaaacttgtccctcagcaaactattttgactgtaaagcaatgtccattggtgctcgttcgcagtcagcacgaacttacttggaaaggcacatgactgaatttactgactgtaatctaaatgagctagttaaacatggactgcgtgccctgagagagactcttcctgctgaacaggatctgacca

(19) United States
(12) Patent Application Publication
(10) Pub. No.: US 2008/0176297 A1
(43) Pub. Date: Jul. 24, 2008

(54) Gene Rcs 21

(76) Inventors: Bastien Chevreux, Rheinfelden (DE); Anne F. Mayer, Basel (CH); Anja Meury, Basel (CH); Nigel J. Mouncey, Binningen (CH); Masako Shinjoh, Kanagawa (JP)

Correspondence Address: NIXON & VANDERHYE, PC, 901 NORTH GLEBE ROAD, 11TH FLOOR, ARLINGTON, VA, 22203, US

(21) Appl. No.: 11/883,723

(22) PCT Filed: Feb. 10, 2006
(86) PCT No.: PCT/EP2006/001228

§ 371 Date: Oct. 22, 2007

(30) Foreign Application Priority Data
Feb. 11, 2005 (EP) 05405066.1
Feb. 11, 2005 (EP) 05405166.9

(51) Int. Cl. C12P 17/04; C12N 15/31; C12N 15/63; C12N 1/21; C07K 14/195; C12N 15/74

(52) U.S. Cl. 435/126; 536/23.7; 435/252.34; 435/252.3; 435/252.33; 435/252.32; 530/324; 435/471

(57) ABSTRACT

The present invention relates to newly identified genes that encode proteins that are involved in the synthesis of L-ascorbic acid (hereinafter also referred to as Vitamin C). The invention also features polynucleotides comprising the full-length polynucleotide sequences of the novel genes and fragments thereof, the novel polypeptides encoded by the polynucleotides and fragments thereof, as well as their functional equivalents. The present invention also relates to the use of said polynucleotides and polypeptides as biotechnological tools in the production of Vitamin C from microorganisms, whereby a modification of said polynucleotides and/or encoded polypeptides has a direct or indirect impact on yield, production, and/or efficiency of production of the fermentation product in said microorganism. Also included are methods/processes of using the polynucleotides and modified polynucleotide sequences to transform host microorganisms. The invention also relates to genetically engineered microorganisms and their use for the direct production of Vitamin C.

The present invention relates to newly identified genes that encode proteins that are involved in the synthesis of L-ascorbic acid (hereinafter also referred to as Vitamin C). The invention also features polynucleotides comprising the full-length polynucleotide sequences of the novel genes and fragments thereof, the novel polypeptides encoded by the polynucleotides and fragments thereof, as well as their functional equivalents. The present invention also relates to the use of said polynucleotides and polypeptides as biotechnological tools in the production of Vitamin C from microorganisms, whereby a modification of said polynucleotides and/or encoded polypeptides has a direct or indirect impact on yield, production, and/or efficiency of production of the fermentation product in said microorganism. Also included are methods/processes of using the polynucleotides and modified polynucleotide sequences to transform host microorganisms. The invention also relates to genetically engineered microorganisms and their use for the direct production of Vitamin C.

Vitamin C is one of very important and indispensable nutrient factors for human beings. Vitamin C is also used in animal feed even though some farm animals can synthesize it in their own body.

For the past 70 years, Vitamin C has been produced industrially from D-glucose by the well-known Reichstein method. All steps in this process are chemical except for one (the conversion of D-sorbitol to L-sorbose), which is carried out by microbial conversion. Since its initial implementation for industrial production of Vitamin C, several chemical and technical modifications have been used to improve the efficiency of the Reichstein method. Recent developments of Vitamin C production are summarized in Ullmann's Encyclopedia of Industrial Chemistry, 5^{th} Edition, Vol. A27 (1996), pp. 547ff.

Different intermediate steps of Vitamin C production have been performed with the help of microorganisms or enzymes isolated therefrom. Thus, 2-keto-L-gulonic acid (2-KGA), an intermediate compound that can be chemically converted into Vitamin C by means of an alkaline rearrangement reaction, may be produced by a fermentation process starting from L-sorbose or D-sorbitol, by means of strains belonging e.g. to the Ketogulonicigenium or Gluconobacter genera, or by an alternative fermentation process NS/10.02.2006 starting from D-glucose, by means of recombinant strains belonging to the Gluconobacter or Pantoea genera.

Current chemical production methods for Vitamin C have some undesirable characteristics such as high-energy consumption and use of large quantities of organic and inorganic solvents. Therefore, over the past decades, other approaches to manufacture Vitamin C using microbial conversions, which would be more economical as well as ecological, have been investigated.

Direct Vitamin C production from a number of substrates including D-sorbitol, L-sorbose and L-sorbosone has been reported in several microorganisms, such as algae, yeast and acetic acid bacteria, using different cultivation methods. Examples of known bacteria able to directly produce Vitamin C include, for instance, strains from the genera of Gluconobacter, Gluconacetobacter, Acetobacter, Ketogulonicigenium, Pantoea, Pseudomonas or Escherichia. Examples of known yeast or algae include, e.g., Candida, Saccharomyces, Zygosaccharomyces, Schizosaccharomyces, Kluyveromyces or Chlorella.

Microorganisms able to assimilate D-sorbitol for growth usually possess enzymes able to oxidize this compound into a universal assimilation substrate such as D-fructose. Also microorganisms able to grow on L-sorbose possess an enzyme, NAD(P)H-dependent L-sorbose reductase, which is able to reduce this compound to D-sorbitol, which is then further oxidized into D-fructose. D-fructose is an excellent substrate for the growth of many microorganisms, after it has been phosphorylated by means of a D-fructose kinase.

For instance, in the case of acetic acid bacteria, which are obligate aerobe, gram-negative microorganisms belonging to the genus Acetobacter, Gluconobacter, and Gluconacetobacter, these microorganisms are able to transport D-sorbitol into the cytosol and convert it into D-fructose by means of a cytosolic NAD-dependent D-sorbitol dehydrogenase. Some individual strains, such as Gluconobacter oxydans IFO 3292, and IFO 3293, are able as well to transport L-sorbose into the cytosol and reduce it to D-sorbitol by means of a cytosolic NAD(P)H-dependent L-sorbose reductase, which then is further oxidized into D-fructose. In these bacteria, the Embden-Meyerhof-Parnas pathway, as well as the tricarboxyclic acid cycle are not fully active, and the main pathway channeling sugars into the central metabolism is the pentose phosphate pathway. D-fructose-6-phosphate, obtained from D-fructose by a phosphorylation reaction enters the pentose phosphate pathway, being further metabolized and producing reducing power in the form of NAD(P)H and tricarboxylic compounds necessary for growth and maintenance.

Acetic acid bacteria are well known for their ability to incompletely oxidize different substrates such as alcohols, sugars, sugar alcohols and aldehydes. These processes are generally known as oxidative fermentations or incomplete oxidations, and they have been well established for a long time in the food and chemical industry, especially in vinegar and in L-sorbose production. A useful product known to be obtained from incomplete oxidations of D-sorbitol or L-sorbose using strains belonging to the Gluconobacter genus is 2-KGA.

Acetic acid bacteria accomplish these incomplete oxidation reactions by means of different dehydrogenases located either in the periplasmic space, on the periplasmic membrane as well as in the cytoplasm. Different co-factors are employed by the different dehydrogenases, the most common being PQQ and FAD for membrane-bound or periplasmic enzymes, and NAD/NADP for cytoplasmic enzymes.

While all products of these oxidation reactions diffuse back to the external aqueous environment through the outer membrane, some of them can be passively or actively transported into the cell and be further used in metabolic pathways responsible for growth and energy formation. Inside the cell, oxidized products can many times be reduced back to their original substrate by means of reductases, and then be channeled back to the central metabolism.

Proteins, in particular enzymes and transporters, that are active in transport of electrons are herein referred to as being involved in the Respiratory Chain System. Such proteins are abbreviated herein as RCS proteins and function in the well-known respiratory chain of an organism, also known as the electron transport system.

RCS proteins are known to be important in the mechanism through which electrons generated by any oxidoreduction reaction in the cell are further transported, in general by means of a series of oxidoreduction reactions involving co-factors and oxidases, and a final electron acceptor.

The main mechanism that living organisms use for producing energy necessary for vital activities is respiration. In higher organisms, carbohydrates, proteins, aliphatic acids are metabolized into acetyl-CoA by means of the glycolysis catabolic pathway and oxidation in cytoplasm. Acetyl-CoA is further metabolized through a series of reactions known as the citric acid cycle, which happens at the mitochondria. Energy resulting from these reactions is used for the production of reducing power, saved in the form of compounds such as FADH₂ and NADH. These compounds are then used in the so-called electron transport chain, a series of oxido-reduction chain reactions involving different components localized in the mitochondrial inner membranes. The final electron acceptor is oxygen, which then reacts with the protons resulting from the reaction chain and forms water. The proton concentration gradient resulting from this process is the driving force of the ATP synthesis.

In bacteria, this basic respiration process follows the same physiologic principle, but can occur in different ways, involving different components, intermediates, enzymatic complexes and final products. The efficiency of bacterial respiration processes can greatly vary, depending on the functional biological components expressed by each species, which in its turn depends on the genetic machinery available and on given growing conditions.

As an example, acetic acid bacteria, which are obligate aerobe, gram-negative microorganisms belonging to the genus Acetobacter, Gluconobacter, and Gluconacetobacter, present peculiar characteristics in terms of energy generating processes. These bacteria are well known for their ability to incompletely oxidize different substrates such as alcohols, sugars, sugar alcohols and aldehydes. These processes are generally known as oxidative fermentations, and they have been well established for a long time in the food and chemical industry, especially in vinegar and in L-sorbose production. Useful products known to be obtained from incomplete oxidations using strains belonging to the Gluconobacter genus are 2-keto-L-gulonic acid (2-KGA) starting from D-sorbitol and L-sorbose, and 5-keto-D-gluconic acid, a precursor for the biosynthesis of D-tartaric acid, starting from D-glucose. Incomplete oxidations are the main mechanism of generation of energy for acetic acid bacteria. They accomplish these reactions by means of different dehydrogenases located either in the periplasmic space, on the periplasmic membrane as well as in the cytoplasm. Different co-factors are employed by the different dehydrogenases, the most common being PQQ and FAD for membrane-bound or periplasmic enzymes, and NAD/NADP for cytoplasmic enzymes. The electron transport chain of Gluconobacter/Gluconacetobacter and Acetobacter strains is known to include co-enzyme Q10 (CoQ10) and CoQ9, respectively, as universal electron transport compound for all processes, as well as in some cases several kinds of cytochrome c elements. Gluconobacter strains are reported not to contain cytochrome c oxidase, but have other kinds of terminal oxidases, such as the bo type.

An object of the present invention is to improve the yields and or productivity of Vitamin C production.

Surprisingly, it has now been found that RCS proteins or subunits of such proteins which are involved in the transport of electrons play an important role in the biotechnological production of Vitamin C.

In one embodiment, RCS proteins of the present invention are selected from oxidoreductases [EC 1], preferably from oxidoreductases acting on diphenols and related substances as donors [EC 1.10], more preferably from oxidoreductases with oxygen as acceptor [EC 1.10.3] and oxidoreductases with other acceptors [EC 1.1.99], most preferably alcohol dehydrogenases (acceptor) [EC 1.1.99.8].

Furthermore, the RCS proteins of the present invention may be selected from respiratory chain proteins, more preferably from carrier proteins or proteins functioning in the biosynthesis of cofactors and/or prosthetic groups, in particular proteins involved in the biosynthesis or maturation of cofactors and/or their precursors such as FAD, NAD, NADP, PQQ, ubiquinones including CoQ8, 9 or CoQ10, cytochromes a, b, c, d, and heme, cyanide-insensitive bd-type terminal oxidase subunits I (CydA) and II (CydB), cyanide-sensitive bo-type terminal oxidase subunits in particular subunit I, II, III and IV, cytochrome c oxidase subunits in particular membrane-bound alcohol dehydrogenase g3-ADH cytochrome c subunit. Most preferably, they are selected from PQQ biosynthetic proteins such as PQQ biosynthetic proteins A, B, C, D, E or from heme exporters such as CcmA or CycW heme exporter.

In particular, it has now been found that RCS proteins encoded by polynucleotides having a nucleotide sequence that hybridizes preferably under highly stringent conditions to a sequence shown in SEQ ID NO:1 play an important role in the biotechnological production of Vitamin C. It has also been found, that by genetically altering the expression level of nucleotides according to the invention in a microorganism capable of directly producing Vitamin C, such as for example Gluconobacter, the direct fermentation of Vitamin C by said microorganism can be even greatly improved.

Consequently, the invention relates to a polynucleotide selected from the group consisting of:

(a) polynucleotides encoding a polypeptide comprising the amino acid sequence according to SEQ ID NO:2;
(b) polynucleotides comprising the nucleotide sequence according to SEQ ID NO:1;
(c) polynucleotides comprising a nucleotide sequence obtainable by nucleic acid amplification such as polymerase chain reaction, using genomic DNA from a microorganism as a template and a primer set according to SEQ ID NO:3 and SEQ ID NO:4;
(d) polynucleotides comprising a nucleotide sequence encoding a fragment or derivative of a polypeptide encoded by a polynucleotide of any of (a) to (c) wherein in said derivative one or more amino acid residues are conservatively substituted compared to said polypeptide, and said fragment or derivative has the activity of PQQ biosynthesis protein (RCS 21);
(e) polynucleotides the complementary strand of which hybridizes under stringent conditions to a polynucleotide as defined in any one of (a) to (d) and which encode a PQQ biosynthesis protein (RCS 21); and
(f) polynucleotides which are at least 70%, such as 85, 90 or 95% identical to a polynucleotide as defined in any one of (a) to (d) and which encode a PQQ biosynthesis protein (RCS 21);
or
the complementary strand of such a polynucleotide.

The RCS protein as isolated from Gluconobacter oxydans DSM 17078 shown in SEQ ID NO:2 and described herein was found to be a particularly useful RCS protein, since it appeared that it performs a crucial function in the direct Vitamin C production in microorganisms, in particular in bacteria, such as acetic acid bacteria, such as Gluconobacter, Acetobacter and Gluconacetobacter. Accordingly, the invention relates to a polynucleotide encoding a polypeptide according to SEQ ID NO:2. This protein may be encoded by a nucleotide sequence as shown in SEQ ID NO:1. The invention therefore also relates to polynucleotides comprising the nucleotide sequence according to SEQ ID NO:1.

The nucleotide and amino acid sequences determined above were used as a “query sequence” to perform a search with Blast2 program (version 2 or BLAST from National Center for Biotechnology [NCBI] against the database PRO SW-SwissProt (full release plus incremental updates). From the searches, the RCS 21 polynucleotide according to SEQ ID NO:1 was annotated as encoding a coenzyme PQQ biosynthesis protein A.

A nucleic acid according to the invention may be obtained by nucleic acid amplification using cDNA, mRNA or alternatively, genomic DNA, as a template and appropriate oligonucleotide primers such as the nucleotide primers according to SEQ ID NO:3 and SEQ ID NO:4 according to standard PCR amplification techniques. The nucleic acid thus amplified may be cloned into an appropriate vector and characterized by DNA sequence analysis.

The template for the reaction may be cDNA obtained by reverse transcription of mRNA prepared from strains known or suspected to comprise a polynucleotide according to the invention. The PCR product may be subcloned and sequenced to ensure that the amplified sequences represent the sequences of a new nucleic acid sequence as described herein, or a functional equivalent thereof.

The PCR fragment may then be used to isolate a full length cDNA clone by a variety of known methods. For example, the amplified fragment may be labeled and used to screen a bacteriophage or cosmid cDNA library. Alternatively, the labeled fragment may be used to screen a genomic library.

Accordingly, the invention relates to polynucleotides comprising a nucleotide sequence obtainable by nucleic acid amplification such as polymerase chain reaction, using DNA such as genomic DNA from a microorganism as a template and a primer set according to SEQ ID NO:3 and SEQ ID NO:4.

The invention also relates to polynucleotides comprising a nucleotide sequence encoding a fragment or derivative of a polypeptide encoded by a polynucleotide as described herein wherein in said derivative one or more amino acid residues are conservatively substituted compared to said polypeptide, and said fragment or derivative has the activity of a RCS polypeptide, preferably a RCS 21 polypeptide.

The invention also relates to polynucleotides the complementary strand of which hybridizes under stringent conditions to a polynucleotide as defined herein and which encode a RCS polypeptide, preferably a RCS 21 polypeptide.

The invention also relates to polynucleotides which are at least 70% identical to a polynucleotide as defined herein and which encode a RCS polypeptide; and the invention also relates to polynucleotides being the complementary strand of a polynucleotide as defined herein above.

The invention also relates to microorganisms wherein the activity of a RCS polypeptide, preferably a RCS 21 polypeptide, is enhanced and/or improved so that the yield of Vitamin C which is directly produced from D-sorbitol or L-sorbose is increased. This may be accomplished, for example, by transferring a polynucleotide according to the invention into a recombinant or non-recombinant microorganism that may or may not contain an endogenous equivalent of the RCS 21 gene.

The skilled person will know how to enhance and/or improve the activity of a RCS protein, preferably a RCS 21 protein. Such may be for instance accomplished by either genetically modifying the host organism in such a way that it produces more or more stable copies of the RCS protein, preferably the RCS 21 protein, than the wild type organism or by increasing the specific activity of the RCS protein, preferably the RCS 21 protein.

In the following description, procedures are detailed to achieve this goal, i.e. the increase in the yield and/or production of Vitamin C which is which is directly produced from D-sorbitol or L-sorbose by increasing the activity of a RCS 21 protein. These procedures apply mutatis mutandis for other RCS proteins.

Modifications in order to have the organism produce more copies of the RCS 21 gene, i.e. overexpressing the gene, and/or protein may include the use of a strong promoter, or the mutation (e.g. insertion, deletion or point mutation) of (parts of) the RCS 21 gene or its regulatory elements. It may also involve the insertion of multiple copies of the gene into a suitable microorganism. An increase in the specific activity of an RCS 21 protein may also be accomplished by methods known in the art. Such methods may include the mutation (e.g. insertion, deletion or point mutation) of (parts of) the RCS 21 gene. A gene is said to be “overexpressed” if the level of transcription of said gene is enhanced in comparison to the wild type gene. This may be measured by for instance Northern blot analysis quantifying the amount of mRNA as an indication for gene expression. As used herein, a gene is overexpressed if the amount of generated mRNA is increased by at least 1%, 2%, 5% 10%, 25%, 50%, 75%, 100%, 200% or even more than 500%, compared to the amount of mRNA generated from a wild-type gene.

Also known in the art are methods of increasing the activity of a given protein by contacting the RCS 21 protein with specific enhancers or other substances that specifically interact with the RCS 21 protein. In order to identify such specific enhancers, the RCS 21 protein may be expressed and tested for activity in the presence of compounds suspected to enhance the activity of the RCS 21 protein. The activity of the RCS 21 protein may also be increased by stabilizing the messenger RNA encoding RCS 21. Such methods are also known in the art, see for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

The invention may be performed in any microorganism carrying a RCS 21 gene or homologue thereof. Suitable microorganisms may be selected from the group consisting of yeast, algae and bacteria, either as wild type strains, mutant strains derived by classic mutagenesis and selection methods or as recombinant strains. Examples of such yeast may be, e.g., Candida, Saccharomyces, Zygosaccharomyces, Schizosaccharomyces, or Kluyveromyces. An example of such algae may be, e.g., Chlorella. Examples of such bacteria may be, e.g., Gluconobacter, Acetobacter, Gluconacetobacter, Ketogulonicigenium, Pantoea, Pseudomonas, such as, e.g., Pseudomonas putida, and Escherichia, such as, e.g., Escherichia coli. Preferred are Gluconobacter or Acetobacter aceti, such as for instance G. oxydans, G. cerinus, G. frateurii, A. aceti subsp. xylinum or A. aceti subsp. orleanus, preferably G. oxydans DSM 17078. Gluconobacter oxydans DSM 17078 (formerly known as Gluconobacter oxydans N44-1) has been deposited at Deutsche Sammlung von Mikroorganismen und Zellkulturen (DSMZ), Mascheroder Weg 1B, D-38124 Braunschweig, Germany according to the Budapest Treaty on 26. January 2005.

Microorganisms which can be used for the present invention may be publicly available from different sources, e.g., Deutsche Sammlung von Mikroorganismen und Zellkulturen (DSMZ), Mascheroder Weg 1B, D-38124 Braunschweig, Germany, American Type Culture Collection (ATCC), P.O. Box 1549, Manassas, Va. 20108 USA or Culture Collection Division, NITE Biological Resource Center, 2-5-8, Kazusakamatari, Kisarazushi, Chiba, 292-0818, Japan (formerly: Institute for Fermentation, Osaka (IFO), 17-85, Juso-honmachi 2-chome, Yodogawa-ku, Osaka 532-8686, Japan). Examples of preferred bacteria deposited with IFO are for instance Gluconobacter oxydans (formerly known as G. melanogenus) IFO 3293, Gluconobacter oxydans (formerly known as G. melanogenus) IFO 3292, Gluconobacter oxydans (formerly known as G. rubiginosus) IFO 3244, Gluconobacter frateurii (formerly known as G. industrius) IFO 3260, Gluconobacter cerinus IFO 3266, Gluconobacter oxydans IFO 3287, and Acetobacter aceti subsp. orleanus IFO 3259, which were all deposited on Apr. 5, 1954; Acetobacter aceti subsp. xylinum IFO 13693 deposited on Oct. 22, 1975, and Acetobacter aceti subsp. xylinum IFO 13773 deposited on Dec. 8, 1977. Strain Acetobacter sp. ATCC 15164, which is also an example of a preferred bacterium, was deposited with ATCC. Strain Gluconobacter oxydans (formerly known as G. melanogenus) N 44-1 as another example of a preferred bacterium is a derivative of the strain IFO 3293 and is described in Sugisawa et al., Agric. Biol. Chem. 54: 1201-1209, 1990.

A microorganism as of the present invention may carry further modifications either on the DNA or protein level (see above), as long as such modification has a direct impact on the yield, production and/or efficiency of the direct production of Vitamin C from substrates like e.g. D-sorbitol or L-sorbose. Such further modifications may for instance affect other genes encoding RCS proteins as described above, in particular genes encoding membrane-bound L-sorbosone dehydrogenases, such as L-sorbosone dehydrogenase SNDHai, or membrane-bound PQQ bound D-sorbitol dehydrogenases. Methods of performing such modifications are known in the art, with some examples further described herein. For the use of SNDHai for direct production of vitamin C as well as the nucleotide and amino acid sequence thereof we refer to WO 2005/017159 which is incorporated herein by reference.

In accordance with a further object of the present invention there is provided the use of a polynucleotide as defined above or a microorganism which is genetically engineered using such polynucleotides in the production of Vitamin C.

The invention also relates to processes for the expression of endogenous genes in a microorganism, to processes for the production of polypeptides as defined above in a microorganism and to processes for the production of microorganisms capable of producing Vitamin C. All these processes may comprise the step of altering a microorganism, wherein “altering” as used herein encompasses the process for “genetically altering” or “altering the composition of the cell culture media and/or methods used for culturing” in such a way that the yield and/or productivity of the fermentation product can be improved compared to the wild-type organism. As used herein, “improved yield of Vitamin C” means an increase of at least 5%, 10%, 25%, 30%, 40%, 50%, 75%, 100%, 200% or even more than 500%, compared to a wild-type microorganism, i.e. a microorganism which is not genetically altered.

The term “genetically engineered” or “genetically altered” means the scientific alteration of the structure of genetic material in a living organism. It involves the production and use of recombinant DNA. More in particular it is used to delineate the genetically engineered or modified organism from the naturally occurring organism. Genetic engineering may be done by a number of techniques known in the art, such as e.g. gene replacement, gene amplification, gene disruption, transfection, transformation using plasmids, viruses, or other vectors. A genetically modified organism, e.g. genetically modified microorganism, is also often referred to as a recombinant organism, e.g. recombinant microorganism.

In accordance with still another aspect of the invention there is provided a process for the production of Vitamin C by direct fermentation.

In particular, the present invention provides a process for the direct production of Vitamin C comprising converting a substrate into Vitamin C. This may for instance be done in a medium comprising a microorganism, which may be a resting or a growing microorganism, preferably a resting microorganism.

Several substrates may be used as a carbon source in a process of the present invention, i.e. a process for direct conversion of a given substrate into Vitamin C such as e.g. mentioned above. Particularly suited carbon sources are those that are easily obtainable from the D-glucose or D-sorbitol metabolization pathway such as, for example, D-glucose, D-sorbitol, L-sorbose, L-sorbosone, 2-keto-L-gulonate, D-gluconate, 2-keto-D-gluconate or 2,5-diketo-gluconate. Preferably, the substrate is selected from for instance D-glucose, D-sorbitol, L-sorbose or L-sorbosone, more preferably from D-glucose, D-sorbitol or L-sorbose, and most preferably from D-sorbitol, L-sorbose or L-sorbosone. The term “substrate” and “production substrate” in connection with the above process using a microorganism is used interchangeably herein.

A medium as used herein for the above process using a microorganism may be any suitable medium for the production of Vitamin C. Typically, the medium is an aqueous medium comprising for instance salts, substrate(s), and a certain pH. The medium in which the substrate is converted into Vitamin C is also referred to as the production medium.

“Fermentation” or “production” or “fermentation process” as used herein may be the use of growing cells using media, conditions and procedures known to the skilled person, or the use of non-growing so-called resting cells, after they have been cultivated by using media, conditions and procedures known to the skilled person, under appropriate conditions for the conversion of suitable substrates into desired products such as Vitamin C. Preferably, resting cells are used for the production of Vitamin C.

The term “direct fermentation”, “direct production”, “direct conversion” and the like is intended to mean that a microorganism is capable of the conversion of a certain substrate into the specified product by means of one or more biological conversion steps, without the need of any additional chemical conversion step. For instance, the term “direct conversion of D-sorbitol into Vitamin C” is intended to describe a process wherein a microorganism is producing Vitamin C and wherein D-sorbitol is offered as a carbon source without the need of an intermediate chemical conversion step. A single microorganism capable of directly fermenting Vitamin C is preferred. Said microorganism is cultured under conditions which allow such conversion from the substrate as defined above.

In connection with the above process using a microorganism it is understood that the above-mentioned microorganisms also include synonyms or basonyms of such species having the same physiological properties, as defined by the International Code of Nomenclature of Prokaryotes. The nomenclature of the microorganisms as used herein is the one officially accepted (at the filing date of the priority application) by the International Committee on Systematics of Prokaryotes and the Bacteriology and Applied Microbiology Division of the International Union of Microbiological Societies, and published by its official publication vehicle International Journal of Systematic and Evolutionary Microbiology (IJSEM). A particular reference is made to Urbance et al., IJSEM (2001) vol 51:1059-1070, with a corrective notification on IJSEM (2001) vol 51:1231-1233, describing the taxonomically reclassification of G. oxydans DSM 4025 as Ketogulonicigenium vulgare.

As used herein, resting cells refer to cells of a microorganism which are for instance viable but not actively growing, or which are growing at low specific growth rates, for instance, growth rates that are lower than 0.02 h⁻¹, preferably lower than 0.01 h⁻¹. Cells which show the above growth rates are said to be in a “resting cell mode”.

The process of the present invention as above using a microorganism may be performed in different steps or phases: preferably, the microorganism is cultured in a first step (also referred to as step (a) or growth phase) under conditions which enable growth. This phase is terminated by changing of the conditions such that the growth rate of the microorganism is reduced leading to resting cells, also referred to as step (b), followed by the production of Vitamin C from the substrate using the (b), also referred to as production phase.

Growth and production phase as performed in the above process using a microorganism may be performed in the same vessel, i.e., only one vessel, or in two or more different vessels, with an optional cell separation step between the two phases. The produced Vitamin C can be recovered from the cells by any suitable means. Recovering means for instance that the produced Vitamin C may be separated from the production medium. Optionally, the thus produced Vitamin C may be further processed.

For the purpose of the present invention relating to the above process using a microorganism, the terms “growth phase”, “growing step”, “growth step” and “growth period” are used interchangeably herein. The same applies for the terms “production phase”, “production step”, “production period”.

One way of performing the above process using a microorganism as of the present invention may be a process wherein the microorganism is grown in a first vessel, the so-called growth vessel, as a source for the resting cells, and at least part of the cells are transferred to a second vessel, the so-called production vessel. The conditions in the production vessel may be such that the cells transferred from the growth vessel become resting cells as defined above. Vitamin C is produced in the second vessel and recovered therefrom.

In connection with the above process using a microorganism, in one aspect, the growing step can be performed in an aqueous medium, i.e. the growth medium, supplemented with appropriate nutrients for growth under aerobic conditions. The cultivation may be conducted, for instance, in batch, fed-batch, semi-continuous or continuous mode. The cultivation period may vary depending on for instance the host, pH, temperature and nutrient medium to be used, and may be for instance about 10 h to about 10 days, preferably about 1 to about 10 days, more preferably about 1 to about 5 days when run in batch or fed-batch mode, depending on the microorganism. If the cells are grown in continuous mode, the residence time may be for instance from about 2 to about 100 h, preferably from about 2 to about 50 h, depending on the microorganism. If the microorganism is selected from bacteria, the cultivation may be conducted for instance at a pH of about 3.0 to about 9.0, preferably about 4.0 to about 9.0, more preferably about 4.0 to about 8.0, even more preferably about 5.0 to about 8.0. If algae or yeast are used, the cultivation may be conducted, for instance, at a pH below about 7.0, preferably below about 6.0, more preferably below about 5.5, and most preferably below about 5.0. A suitable temperature range for carrying out the cultivation using bacteria may be for instance from about 13° C. to about 40° C., preferably from about 18° C. to about 37° C., more preferably from about 13° C. to about 36° C., and most preferably from about 18° C. to about 33° C. If algae or yeast are used, a suitable temperature range for carrying out the cultivation may be for instance from about 15° C. to about 40° C., preferably from about 20° C. to about 45° C., more preferably from about 25° C. to about 40° C., even more preferably from about 25° C. to about 38° C., and most preferably from about 30° C. to about 38° C. The culture medium for growth usually may contain such nutrients as assimilable carbon sources, e.g., glycerol, D-mannitol, D-sorbitol, L-sorbose, erythritol, ribitol, xylitol, arabitol, inositol, dulcitol, D-ribose, D-fructose, D-glucose, sucrose, and ethanol, preferably L-sorbose, D-glucose, D-sorbitol, D-mannitol, glycerol and ethanol; and digestible nitrogen sources such as organic substances, e.g., peptone, yeast extract and amino acids. The media may be with or without urea and/or corn steep liquor and/or baker's yeast. Various inorganic substances may also be used as nitrogen sources, e.g., nitrates and ammonium salts. Furthermore, the growth medium, usually may contain inorganic salts, e.g., magnesium sulfate, manganese sulfate, potassium phosphate, and calcium carbonate. Cells obtained using the procedures described above can then be further incubated at essentially the same modes, temperature and pH conditions as described above, in the presence of substrates such as D-sorbitol, L-sorbose, or D-glucose, in such a way that they convert these substrates directly into Vitamin C. Incubation can be done in a nitrogen-rich medium, containing, for example, organic nitrogen sources, e.g., peptone, yeast extract, baker's yeast, urea, amino acids, and corn steep liquor, or inorganic nitrogen sources, e.g., nitrates and ammonium salts, in which case cells will be able to further grow while producing Vitamin C. Alternatively, incubation can be done in a nitrogen-poor medium, in which case cells will not grow substantially, and will be in a resting cell mode, or biotransformation mode. In all cases, the incubation medium may also contain inorganic salts, e.g., magnesium sulfate, manganese sulfate, potassium phosphate, and calcium chloride.

In connection with the above process using a microorganism, in the growth phase the specific growth rates are for instance at least 0.02 h⁻¹. For cells growing in batch, fed-batch or semi-continuous mode, the growth rate depends on for instance the composition of the growth medium, pH, temperature, and the like. In general, the growth rates may be for instance in a range from about 0.05 to about 0.2 h⁻¹, preferably from about 0.06 to about 0.15 h⁻¹, and most preferably from about 0.07 to about 0.13 h⁻¹.

In another aspect of the above process using a microorganism, resting cells may be provided by cultivation of the respective microorganism on agar plates thus serving as growth vessel, using essentially the same conditions, e.g., cultivation period, pH, temperature, nutrient medium as described above, with the addition of agar agar.

In connection with the above process using a microorganism, if the growth and production phase are performed in two separate vessels, then the cells from the growth phase may be harvested or concentrated and transferred to a second vessel, the so-called production vessel. This vessel may contain an aqueous medium supplemented with any applicable production substrate that can be converted to Vitamin C by the cells. Cells from the growth vessel can be harvested or concentrated by any suitable operation, such as for instance centrifugation, membrane crossflow ultrafiltration or microfiltration, filtration, decantation, flocculation. The cells thus obtained may also be transferred to the production vessel in the form of the original broth from the growth vessel, without being harvested, concentrated or washed, i.e. in the form of a cell suspension. In a preferred embodiment, the cells are transferred from the growth vessel to the production vessel in the form of a cell suspension without any washing or isolating step in-between.

Thus, in a preferred embodiment of the above process using a microorganism step (a) and (c) of the process of the present invention as described above are not separated by any washing and/or separation step.

In connection with the above process using a microorganism, if the growth and production phase are performed in the same vessel, cells may be grown under appropriate conditions to the desired cell density followed by a replacement of the growth medium with the production medium containing the production substrate. Such replacement may be, for instance, the feeding of production medium to the vessel at the same time and rate as the withdrawal or harvesting of supernatant from the vessel. To keep the resting cells in the vessel, operations for cell recycling or retention may be used, such as for instance cell recycling steps. Such recycling steps, for instance, include but are not limited to methods using centrifuges, filters, membrane crossflow microfiltration of ultrafiltration steps, membrane reactors, flocculation, or cell immobilization in appropriate porous, non-porous or polymeric matrixes. After a transition phase, the vessel is brought to process conditions under which the cells are in a resting cell mode as defined above, and the production substrate is efficiently converted into Vitamin C.

The aqueous medium in the production vessel as used for the production step in connection with the above process using a microorganism, hereinafter called production medium, may contain only the production substrate(s) to be converted into Vitamin C, or may contain for instance additional inorganic salts, e.g., sodium chloride, calcium chloride, magnesium sulfate, manganese sulfate, potassium phosphate, calcium phosphate, and calcium carbonate. The production medium may also contain digestible nitrogen sources such as for instance organic substances, e.g., peptone, yeast extract, urea, amino acids, and corn steep liquor, and inorganic substances, e.g. ammonia, ammonium sulfate, and sodium nitrate, at such concentrations that the cells are kept in a resting cell mode as defined above. The medium may be with or without urea and/or corn steep liquor and/or baker's yeast. The production step may be conducted for instance in batch, fed-batch, semi-continuous or continuous mode. In case of fed-batch, semi-continuous or continuous mode, both cells from the growth vessel and production medium can be fed continuously or intermittently to the production vessel at appropriate feed rates. Alternatively, only production medium may be fed continuously or intermittently to the production vessel, while the cells coming from the growth vessel are transferred at once to the production vessel. The cells coming from the growth vessel may be used as a cell suspension within the production vessel or may be used as for instance flocculated or immobilized cells in any solid phase such as porous or polymeric matrixes. The production period, defined as the period elapsed between the entrance of the substrate into the production vessel and the harvest of the supernatant containing Vitamin C, the so-called harvest stream, can vary depending for instance on the kind and concentration of cells, pH, temperature and nutrient medium to be used, and is preferably about 2 to about 100 h. The pH and temperature can be different from the pH and temperature of the growth step, but is essentially the same as for the growth step.

In a preferred embodiment of the above process using a microorganism, the production step is conducted in continuous mode, meaning that a first feed stream containing the cells from the growth vessel and a second feed stream containing the substrate is fed continuously or intermittently to the production vessel. The first stream may either contain only the cells isolated/separated from the growth medium or a cell suspension, coming directly from the growth step, i.e. cells suspended in growth medium, without any intermediate step of cell separation, washing and/or isolating. The second feed stream as herein defined may include all other feed streams necessary for the operation of the production step, e.g. the production medium comprising the substrate in the form of one or several different streams, water for dilution, and base for pH control.

In connection with the above process using a microorganism, when both streams are fed continuously, the ratio of the feed rate of the first stream to feed rate of the second stream may vary between about 0.01 and about 10, preferably between about 0.01 and about 5, most preferably between about 0.02 and about 2. This ratio is dependent on the concentration of cells and substrate in the first and second stream, respectively.

Another way of performing the process as above using a microorganism of the present invention may be a process using a certain cell density of resting cells in the production vessel. The cell density is measured as absorbance units (optical density) at 600 nm by methods known to the skilled person. In a preferred embodiment, the cell density in the production step is at least about 10, more preferably between about 10 and about 200, even more preferably between about 15 and about 200, even more preferably between about 15 to about 120, and most preferably between about 20 and about 120.

In connection with the above process using a microorganism, in order to keep the cells in the production vessel at the desired cell density during the production phase as performed, for instance, in continuous or semi-continuous mode, any means known in the art may be used, such as for instance cell recycling by centrifugation, filtration, membrane crossflow ultrafiltration of microfiltration, decantation, flocculation, cell retention in the vessel by membrane devices or cell immobilization. Further, in case the production step is performed in continuous or semi-continuous mode and cells are continuously or intermittently fed from the growth vessel, the cell density in the production vessel may be kept at a constant level by, for instance, harvesting an amount of cells from the production vessel corresponding to the amount of cells being fed from the growth vessel.

In connection with the above process using a microorganism, the produced Vitamin C contained in the so-called harvest stream is recovered/harvested from the production vessel. The harvest stream may include, for instance, cell-free or cell-containing aqueous solution coming from the production vessel, which contains Vitamin C as a result of the conversion of production substrate by the resting cells in the production vessel. Cells still present in the harvest stream may be separated from the Vitamin C by any operations known in the art, such as for instance filtration, centrifugation, decantation, membrane crossflow ultrafiltration or microfiltration, tangential flow ultrafiltration or microfiltration or dead end filtration. After this cell separation operation, the harvest stream is essentially free of cells.

In a further aspect, the process of the present invention may be combined with further steps of separation and/or purification of the produced Vitamin C from other components contained in the harvest stream, i.e., so-called downstream processing steps. These steps may include any means known to a skilled person, such as, for instance, concentration, crystallization, precipitation, adsorption, ion exchange, electrodialysis, bipolar membrane electrodialysis and/or reverse osmosis. Vitamin C may be further purified as the free acid form or any of its known salt forms by means of operations such as for instance treatment with activated carbon, ion exchange, adsorption and elution, concentration, crystallization, filtration and drying. Specifically, a first separation of Vitamin C from other components in the harvest stream might be performed by any suitable combination or repetition of, for instance, the following methods: two- or three-compartment electrodialysis, bipolar membrane electrodialysis, reverse osmosis or adsorption on, for instance, ion exchange resins or non-ionic resins. If the resulting form of Vitamin C is a salt of L-ascorbic acid, conversion of the salt form into the free acid form may be performed by for instance bipolar membrane electrodialysis, ion exchange, simulated moving bed chromatographic techniques, and the like. Combination of the mentioned steps, e.g., electrodialysis and bipolar membrane electrodialysis into one step might be also used as well as combination of the mentioned steps e.g. several steps of ion exchange by using simulated moving bed chromatographic methods. Any of these procedures alone or in combination constitute a convenient means for isolating and purifying the product, i.e. Vitamin C. The product thus obtained may further be isolated in a manner such as, e.g. by concentration, crystallization, precipitation, washing and drying of the crystals and/or further purified by, for instance, treatment with activated carbon, ion exchange and/or re-crystallization.

In a preferred embodiment, Vitamin C is purified from the harvest stream by a series of downstream processing steps as described above without having to be transferred to a non-aqueous solution at any time of this processing, i.e. all steps are performed in an aqueous environment. Such preferred downstream processing procedure may include for instance the concentration of the harvest stream coming from the production vessel by means of two- or three-compartment electrodialysis, conversion of Vitamin C in its salt form present in the concentrated solution into its acid form by means of bipolar membrane electrodialysis and/or ion exchange, purification by methods such as for instance treatment with activated carbon, ion exchange or non-ionic resins, followed by a further concentration step and crystallization. These crystals can be separated, washed and dried. If necessary, the crystals may be again re-solubilized in water, treated with activated carbon and/or ion exchange resins and recrystallized. These crystals can then be separated, washed and dried.

Advantageous embodiments of the invention become evident from the dependent claims. These and other aspects and embodiments of the present invention should be apparent to those skilled in the art from the teachings herein.

The sequence of the gene comprising a nucleotide sequence according to SEQ ID NO:1 encoding a RCS 21 protein was determined by sequencing a genomic clone obtained from Gluconobacter oxydans DSM 17078.

The invention also relates to a polynucleotide encoding at least a biologically active fragment or derivative of a RCS 21 polypeptide as shown in SEQ ID NO:2.

As used herein, “biologically active fragment or derivative” means a polypeptide which retains essentially the same biological function or activity as the polypeptide shown in SEQ ID NO:2. Examples of biological activity may for instance be enzymatic activity, signaling activity or antibody reactivity. The term “same biological function” or “functional equivalent” as used herein means that the protein has essentially the same biological activity, e.g. enzymatic, signaling or antibody reactivity, as a polypeptide shown in SEQ ID NO:2.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally-occurring polynucleotide or polypeptide present in a living microorganism is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

An isolated polynucleotide or nucleic acid as used herein may be a DNA or RNA that is not immediately contiguous with both of the coding sequences with which it is immediately contiguous (one on the 5′-end and one on the 3′-end) in the naturally occurring genome of the organism from which it is derived. Thus, in one embodiment, a nucleic acid includes some or all of the 5′-non-coding (e.g., promoter) sequences that are immediately contiguous to the coding sequence. The term “isolated polynucleotide” therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide that is substantially free of cellular material, viral material, or culture medium (when produced by recombinant DNA techniques), or chemical precursors or other chemicals (when chemically synthesized). Moreover, an “isolated nucleic acid fragment” is a nucleic acid fragment that is not naturally occurring as a fragment and would not be found in the natural state.

As used herein, the terms “polynucleotide”, “gene” and “recombinant gene” refer to nucleic acid molecules which may be isolated from chromosomal DNA, which include an open reading frame encoding a protein, e.g. G. oxydans DSM 17078 RCS proteins. A polynucleotide may include a polynucleotide sequence as shown in SEQ ID NO:1 or fragments thereof and regions upstream and downstream of the gene sequences which may include, for example, promoter regions, regulator regions and terminator regions important for the appropriate expression and stabilization of the polypeptide derived thereof.

A gene may include coding sequences, non-coding sequences such as for instance untranslated sequences located at the 3′- and 5′-ends of the coding region of a gene, and regulatory sequences. Moreover, a gene refers to an isolated nucleic acid molecule as defined herein. It is furthermore appreciated by the skilled person that DNA sequence polymorphisms that lead to changes in the amino acid sequences of RCS proteins may exist within a population, e.g., the Gluconobacter oxydans population. Such genetic polymorphism in the RCS 21 gene may exist among individuals within a population due to natural variation or in cells from different populations. Such natural variations can typically result in 1-5% variance in the nucleotide sequence of the RCS 21 gene. Any and all such nucleotide variations and the resulting amino acid polymorphism in RCS 21 are the result of natural variation and that do not alter the functional activity of RCS proteins are intended to be within the scope of the invention.

As used herein, the terms “polynucleotide” or “nucleic acid molecule” are intended to include DNA molecules (e.g., cDNA or genomic DNA) and RNA molecules (e.g., mRNA) and analogs of the DNA or RNA generated using nucleotide analogs. The nucleic acid molecule may be single-stranded or double-stranded, but preferably is double-stranded DNA. The nucleic acid may be synthesized using oligonucleotide analogs or derivatives (e.g., inosine or phosphorothioate nucleotides). Such oligonucleotides may be used, for example, to prepare nucleic acids that have altered base-pairing abilities or increased resistance to nucleases.

The sequence information as provided herein should not be so narrowly construed as to require inclusion of erroneously identified bases. The specific sequences disclosed herein may be readily used to isolate the complete gene from a recombinant or non-recombinant microorganism capable of converting a given carbon source directly into Vitamin C, in particular Gluconobacter oxydans, preferably Gluconobacter oxydans DSM 17078 which in turn may easily be subjected to further sequence analyses thereby identifying sequencing errors.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence may be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

A nucleic acid molecule according to the invention may comprise only a portion or a fragment of the nucleic acid sequence provided by the present invention, such as for instance the sequence shown in SEQ ID NO:1, for example a fragment which may be used as a probe or primer such as for instance SEQ ID NO:3 or SEQ ID NO:4 or a fragment encoding a portion of a protein according to the invention. The nucleotide sequence determined from the cloning of the RCS 21 gene allows for the generation of probes and primers designed for use in identifying and/or cloning other RCS 21 family members, as well as RCS 21 homologues from other species. The probe/primer typically comprises substantially purified oligonucleotides which typically comprises a region of nucleotide sequence that hybridizes preferably under highly stringent conditions to at least about 12 or 15, preferably about 18 or 20, more preferably about 22 or 25, even more preferably about 30, 35, 40, 45, 50, 55, 60, 65, or 75 or more consecutive nucleotides of a nucleotide sequence shown in SEQ ID NO:1 or a fragment or derivative thereof.

A nucleic acid molecule encompassing all or a portion of the nucleic acid sequence of SEQ ID NO:1 may be also isolated by the polymerase chain reaction (PCR) using synthetic oligonucleotide primers designed based upon the sequence information contained herein.

A nucleic acid of the invention may be amplified using cDNA, mRNA or alternatively, genomic DNA, as a template and appropriate oligonucleotide primers according to standard PCR amplification techniques. The nucleic acid thus amplified may be cloned into an appropriate vector and characterized by DNA sequence analysis.

Fragments of a polynucleotide according to the invention may also comprise polynucleotides not encoding functional polypeptides. Such polynucleotides may function as probes or primers for a PCR reaction.

Nucleic acids according to the invention irrespective of whether they encode functional or non-functional polypeptides, may be used as hybridization probes or polymerase chain reaction (PCR) primers. Uses of the nucleic acid molecules of the present invention that do not encode a polypeptide having a RCS 21 activity include, inter alia, (1) isolating the gene encoding the protein of the present invention, or allelic variants thereof from a cDNA library, e.g., from other organisms than Gluconobacter oxydans and (2) Northern blot analysis for detecting expression of mRNA of said protein in specific cells or (3) use in enhancing and/or improving the function or activity of homologous RCS 21 genes in said other organisms.

Probes based on the nucleotide sequences provided herein may be used to detect transcripts or genomic sequences encoding the same or homologous proteins for instance in other organisms. Nucleic acid molecules corresponding to natural variants and non-G. oxydans homologues of the G. oxydans RCS 21 DNA of the invention which are also embraced by the present invention may be isolated based on their homology to the G. oxydans RCS 21 nucleic acid disclosed herein using the G. oxydans DNA, or a portion thereof, as a hybridization probe according to standard hybridization techniques, preferably under highly stringent hybridization conditions.

In preferred embodiments, the probe further comprises a label group attached thereto, e.g., the label group can be a radioisotope, a fluorescent compound, an enzyme, or an enzyme cofactor.

Homologous gene sequences may be isolated, for example, by performing PCR using two degenerate oligonucleotide primer pools designed on the basis of nucleotide sequences as taught herein.

The template for the reaction may be cDNA obtained by reverse transcription of mRNA prepared from strains known or suspected to express a polynucleotide according to the invention. The PCR product may be subcloned and sequenced to ensure that the amplified sequences represent the sequences of a new nucleic acid sequence as described herein, or a functional equivalent thereof.

The PCR fragment may then be used to isolate a full length cDNA clone by a variety of known methods. For example, the amplified fragment may be labeled and used to screen a bacteriophage or cosmid cDNA library. Alternatively, the labeled fragment may be used to screen a genomic library.

PCR technology can also be used to isolate full-length cDNA sequences from other organisms. For example, RNA may be isolated, following standard procedures, from an appropriate cellular or tissue source. A reverse transcription reaction may be performed on the RNA using an oligonucleotide primer specific for the most 5′-end of the amplified fragment for the priming of first strand synthesis.

The resulting RNA/DNA hybrid may then be “tailed” (e.g., with guanines) using a standard terminal transferase reaction, the hybrid may be digested with RNaseH, and second strand synthesis may then be primed (e.g., with a poly-C primer). Thus, cDNA sequences upstream of the amplified fragment may easily be isolated. For a review of useful cloning strategies, see e.g., Sambrook et al., supra; and Ausubel et al., supra.

Also, nucleic acids encoding other RCS 21 family members, which thus have a nucleotide sequence that differs from a nucleotide sequence according to SEQ ID NO:1, are within the scope of the invention. Moreover, nucleic acids encoding RCS 21 proteins from different species which thus may have a nucleotide sequence which differs from a nucleotide sequence shown in SEQ ID NO:1 are within the scope of the invention.

The invention also relates to an isolated polynucleotide hybridisable under stringent conditions, preferably under highly stringent conditions, to a polynucleotide as of the present invention, such as for instance a polynucleotide shown in SEQ ID NO:1. Advantageously, such polynucleotide may be obtained from a microorganism capable of converting a given carbon source directly into Vitamin C, in particular Gluconobacter oxydans, preferably Gluconobacter oxydans DSM 17078.

As used herein, the term “hybridizing” is intended to describe conditions for hybridization and washing under which nucleotide sequences at least about 50%, at least about 60%, at least about 70%, more preferably at least about 80%, even more preferably at least about 85% to 90%, most preferably at least 95% homologous to each other typically remain hybridized to each other.

In one embodiment, a nucleic acid of the invention is at least 40%, 45%, 50%, 55%, 60%, 65%, 70%, 75%, 80%, 85%, 90%, 91%, 92%, 93%, 94%, 95%, 96%, 97%, 98%, 99%, or more homologous to a nucleic acid sequence shown in SEQ ID NO:1 or the complement thereof.

A preferred, non-limiting example of stringent hybridization conditions are hybridization in 6× sodium chloride/sodium citrate (SSC) at about 45° C., followed by one or more washes in 1×SSC, 0.1% SDS at 50° C., preferably at 55° C., more preferably at 60° C. and even more preferably at 65° C.

Highly stringent conditions include incubations at 42° C. for a period of several days, such as 2-4 days, using a labeled DNA probe, such as a digoxigenin (DIG)-labeled DNA probe, followed by one or more washes in 2×SSC, 0.1% SDS at room temperature and one or more washes in 0.5×SSC, 0.1% SDS or 0.1×SSC, 0.1% SDS at 65-68° C. In particular, highly stringent conditions include, for example, 2 h to 4 days incubation at 42° C. using a DIG-labeled DNA probe (prepared by e.g. using a DIG labeling system; Roche Diagnostics GmbH, 68298 Mannheim, Germany) in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 minutes in 2×SSC and 0.1% SDS at room temperature and then washing twice for 15-30 minutes in 0.5×SSC and 0.1% SDS or 0.1×SSC and 0.1% SDS at 65-68° C.

Preferably, an isolated nucleic acid molecule of the invention that hybridizes under preferably highly stringent conditions to a nucleotide sequence of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural G. oxydans RCS 21 protein.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.). Of course, a polynucleotide which hybridizes only to a poly (A) sequence (such as the 3′-terminal poly (A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

In a typical approach, genomic DNA or cDNA libraries constructed from other organisms, e.g. microorganisms capable of converting a given carbon source directly into Vitamin C, in particular other Gluconobacter species may be screened.

For example, Gluconobacter strains may be screened for homologous polynucleotides by Southern and/or Northern blot analysis. Upon detection of transcripts homologous to polynucleotides according to the invention, DNA libraries may be constructed from RNA isolated from the appropriate strain, utilizing standard techniques well known to those of skill in the art. Alternatively, a total genomic DNA library may be screened using a probe hybridisable to a polynucleotide according to the invention.

A nucleic acid molecule of the present invention, such as for instance a nucleic acid molecule shown in SEQ ID NO:1 or a fragment or derivative thereof, may be isolated using standard molecular biology techniques and the sequence information provided herein. For example, using all or portion of the nucleic acid sequence shown in SEQ ID NO:1 as a hybridization probe, nucleic acid molecules according to the invention may be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Furthermore, oligonucleotides corresponding to or hybridisable to nucleotide sequences according to the invention may be prepared by standard synthetic techniques, e.g., using an automated DNA synthesizer.

The terms “homology” or “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available at http://www.accelrys.com), using either a Blossom 62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available at http://www.accelrys.com), using a NWSgapdna.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (CABIOS, 4: 11-17 (1989) which has been incorporated into the ALIGN program (version 2.0) (available at http://vega.igh.cnrs.fr/bin/align-guess.cgi) using a PAM120 weight residue table, a gap length penalty of 12 and a gap penalty of 4.

The nucleic acid and protein sequences of the present invention may further be used as a “query sequence” to perform a search against public databases to, for example, identify other family members or related sequences. Such searches may be performed using the BLASTN and BLASTX programs (version 2.0) of Altschul, et al. (1990) J. Mol. Biol. 215:403-10. BLAST nucleotide searches may be performed with the BLASTN program, score=100, word length=12 to obtain nucleotide sequences homologous to the nucleic acid molecules of the present invention. BLAST protein searches may be performed with the BLASTX program, score=50, word length=3 to obtain amino acid sequences homologous to the protein molecules of the present invention. To obtain gapped alignments for comparison purposes, Gapped BLAST may be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) may be used. See http://www.ncbi.nlm.nih.gov.

In another preferred embodiment, an isolated nucleic acid molecule of the invention comprises a nucleic acid molecule which is the complement of a nucleotide sequence as of the present invention, such as for instance the sequence shown in SEQ ID NO:1. A nucleic acid molecule, which is complementary to a nucleotide sequence disclosed herein, is one that is sufficiently complementary to a nucleotide sequence shown in SEQ ID NO:1 such that it may hybridize to said nucleotide sequence thereby forming a stable duplex.

In a further preferred embodiment, a nucleic acid of the invention as shown in SEQ ID NO:1 or the complement thereof contains at least one mutation leading to a gene product with modified function/activity. The at least one mutation may be introduced by methods described herein. In one aspect, the at least one mutation leads to a RCS 21 protein whose function and/or activity compared to the wild type counterpart is enhanced or improved. Methods for introducing such mutations are well known in the art.

The term “increase” of activity as used herein encompasses increasing activity of one or more polypeptides in the producing organism, which in turn are encoded by the corresponding polynucleotides described herein. There are a number of methods available in the art to accomplish increase of activity of a given protein, in this case the RCS 21 protein. In general, the specific activity of a protein may be increased or the copy number of the protein may be increased. The term increase of activity or equivalent expressions also encompasses the situation wherein RCS 21 protein activity is introduced in a cell that did not contain this activity before, e.g. by introducing a gene encoding RCS 21 in a cell that did not contain an equivalent of this gene before, or that could not express an active form of the corresponding protein before.

To facilitate such an increase, the copy number of the genes corresponding to the polynucleotides described herein may be increased. Alternatively, a strong promoter may be used to direct the expression of the polynucleotide. In another embodiment, the promoter, regulatory region and/or the ribosome binding site upstream of the gene can be altered to increase the expression. The expression may also be enhanced or increased by increasing the relative half-life of the messenger RNA. In another embodiment, the activity of the polypeptide itself may be increased by employing one or more mutations in the polypeptide amino acid sequence, which increase the activity. For example, altering the affinity of the polypeptide for its corresponding substrate may result in improved activity. Likewise, the relative half-life of the polypeptide may be increased. In either scenario, that being enhanced gene expression or increased specific activity, the improvement may be achieved by altering the composition of the cell culture media and/or methods used for culturing. “Enhanced expression” or “improved activity” as used herein means an increase of at least 5%, 10%, 25%, 50%, 75%, 100%, 200% or even more than 500%, compared to a wild-type protein, polynucleotide, gene; or the activity and/or the concentration of the protein present before the polynucleotides or polypeptides are enhanced and/or improved. The activity of the RCS 21 protein may also be enhanced by contacting the protein with a specific or general enhancer of its activity.

Another aspect of the invention pertains to vectors, containing a nucleic acid encoding a protein according to the invention or a functional equivalent or portion thereof. As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication). Other vectors are integrated into the genome of a host cell upon introduction into the host cell, and thereby are replicated along with the host genome.

Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., attenuator). Such regulatory sequences are described, for example, in Goeddel; Gene Expression Technology: Methods in Enzymology 185, Academic Press, San Diego, Calif. (1990). Regulatory sequences include those which direct constitutive or inducible expression of a nucleotide sequence in many types of host cells and those which direct expression of the nucleotide sequence only in a certain host cell (e.g. tissue-specific regulatory sequences). It will be appreciated by those skilled in the art that the design of the expression vector can depend on such factors as the choice of the host cell to be transformed, the level of expression of protein desired, etc. The expression vectors of the invention may be introduced into host cells to thereby produce proteins or peptides, encoded by nucleic acids as described herein, including, but not limited to, mutant proteins, fragments thereof, variants or functional equivalents thereof, and fusion proteins, encoded by a nucleic acid as described herein, e.g., RCS 21 proteins, mutant forms of RCS 21 proteins, fusion proteins and the like.

The recombinant expression vectors of the invention may be designed for expression of RCS 21 proteins in a suitable microorganism. For example, a protein according to the invention may be expressed in bacterial cells such as strains belonging to the genera Gluconobacter, Gluconacetobacter or Acetobacter. Expression vectors useful in the present invention include chromosomal-, episomal- and virus-derived vectors e.g., vectors derived from bacterial plasmids, bacteriophage, and vectors derived from combinations thereof, such as those derived from plasmid and bacteriophage genetic elements, such as cosmids and phagemids.

The DNA insert may be operatively linked to an appropriate promoter, which may be either a constitutive or inducible promoter. The skilled person will know how to select suitable promoters. The expression constructs may contain sites for transcription initiation, termination, and, in the transcribed region, a ribosome binding site for translation. The coding portion of the mature transcripts expressed by the constructs may preferably include an initiation codon at the beginning and a termination codon appropriately positioned at the end of the polypeptide to be translated.

Vector DNA may be introduced into suitable host cells via conventional transformation or transfection techniques. As used herein, the terms “transformation”, “transconjugation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, transduction, infection, lipofection, cationic lipidmediated transfection or electroporation. Suitable methods for transforming or transfecting host cells may be found in Sambrook, et al. (supra), Davis et al., Basic Methods in Molecular Biology (1986) and other laboratory manuals.

In order to identify and select cells which have integrated the foreign DNA into their genome, a gene that encodes a selectable marker (e.g., resistance to antibiotics) is generally introduced into the host cells along with the gene of interest. Preferred selectable markers include those that confer resistance to drugs, such as kanamycin, tetracycline, ampicillin and streptomycin. A nucleic acid encoding a selectable marker is preferably introduced into a host cell on the same vector as that encoding a protein according to the invention or can be introduced on a separate vector such as, for example, a suicide vector, which cannot replicate in the host cells. Cells stably transfected with the introduced nucleic acid can be identified by drug selection (e.g., cells that have incorporated the selectable marker gene will survive, while the other cells die).

The invention provides also an isolated polypeptide having the amino acid sequence shown in SEQ ID NO:2 or an amino acid sequence obtainable by expressing a polynucleotide of the present invention, such as for instance a polynucleotide sequence shown in SEQ ID NO:1 in an appropriate host.

Polypeptides according to the invention may contain only conservative substitutions of one or more amino acids in the amino acid sequence represented by SEQ ID NO:2 or substitutions, insertions or deletions of non-essential amino acids. Accordingly, a non-essential amino acid is a residue that may be altered in the amino acid sequences shown in SEQ ID NO:2 without substantially altering the biological function. For example, amino acid residues that are conserved among the proteins of the present invention, are predicted to be particularly unamenable to alteration. Furthermore, amino acids conserved among the proteins according to the present invention and other RCS 21 proteins are not likely to be amenable to alteration.

The term “conservative substitution” is intended to mean that a substitution in which the amino acid residue is replaced with an amino acid residue having a similar side chain. These families are known in the art and include amino acids with basic side chains (e.g., lysine, arginine and histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As mentioned above, the polynucleotides of the invention may be utilized in the genetic engineering of a suitable host cell to make it better and more efficient in the fermentation, for example in a direct fermentation process for Vitamin C.

According to the invention a genetically engineered/recombinantly produced host cell (also referred to as recombinant cell or transformed cell) carrying such a modified polynucleotide wherein the function of the linked protein is significantly modified in comparison to a wild-type cell such that the yield, production and/or efficiency of production of one or more fermentation products such as Vitamin C is improved. The host cell may be selected from a microorganism capable of directly producing one or more fermentation products such as for instance Vitamin C from a given carbon source, in particular Gluconobacter oxydans, preferably G. oxydans DSM 17078.

A “transformed cell” or “recombinant cell” is a cell into which (or into an ancestor of which) has been introduced, by means of recombinant DNA techniques, a nucleic acid according to the invention, or wherein the activity of the RCS 21 protein has been increased and/or enhanced. Suitable host cells include cells of microorganisms capable of producing a given fermentation product, e.g., converting a given carbon source directly into Vitamin C. In particular, these include strains from the genera Pseudomonas, Pantoea, Escherichia, Corynebacterium, Ketogulonicigenium and acetic acid bacteria like e.g., Gluconobacter, Acetobacter or Gluconacetobacter, preferably Acetobacter sp., Acetobacter aceti, Gluconobacter frateurii, Gluconobacter cerinus, Gluconobacter thailandicus, Gluconobacter oxydans, more preferably G. oxydans, most preferably G. oxydans DSM 17078.

Improved gene expression may also be achieved by modifying the RCS 21 gene, e.g., by introducing one or more mutations into the RCS 21 gene wherein said modification leads to a RCS 21 protein with a function which is significantly improved in comparison to the wild-type protein.

Therefore, in one other embodiment, the polynucleotide carrying the at least one mutation is derived from a polynucleotide as represented by SEQ ID NO:1 or equivalents thereof.

A mutation as used herein may be any mutation leading to a more functional or more stable polypeptide, e.g. more functional or more stable RCS 21 gene products. This may include for instance an alteration in the genome of a microorganism, which improves the synthesis of RCS 21 or leads to the expression of a RCS 21 protein with an altered amino acid sequence whose function compared with the wild type counterpart having a non-altered amino acid sequence is improved and/or enhanced. The improvement may occur at the transcriptional, translational or post-translational level.

The alteration in the genome of the microorganism may be obtained e.g. by replacing through a single or double crossover recombination a wild type DNA sequence by a DNA sequence containing the alteration. For convenient selection of transformants of the microorganism with the alteration in its genome the alteration may, e.g. be a DNA sequence encoding an antibiotic resistance marker or a gene complementing a possible auxotrophy of the microorganism. Mutations include, but are not limited to, deletion-insertion mutations.

An alteration in the genome of the microorganism leading to a more functional polypeptide may also be obtained by randomly mutagenizing the genome of the microorganism using e.g. chemical mutagens, radiation or transposons and selecting or screening for mutants which are better or more efficient producers of one or more fermentation products. Standard methods for screening and selection are known to the skilled person.

In a specific embodiment, it is desired to knockout or suppress a repressor of the RCS 21 gene of the present invention, i.e., wherein its repressor gene expression is artificially suppressed in order to improve the yield, productivity, and/or efficiency of production of the fermentation product when introduced into a suitable host cell. Methods of providing knockouts as well as microorganisms carrying such suppressed genes are well known in the art. The suppression of the repressor gene may be induced by deleting at least a part of the repressor gene or the regulatory region thereof. As used herein, “suppression of the gene expression” includes complete and partial suppression, as well as suppression under specific conditions and also suppression of the expression of either one of the two alleles.

The aforementioned mutagenesis strategies for RCS 21 proteins may result in increased yields of a desired compound in particular Vitamin C. This list is not meant to be limiting; variations on these mutagenesis strategies will be readily apparent to one of ordinary skill in the art. By these mechanisms, the nucleic acid and protein molecules of the invention may be utilized to generate microorganisms such as Gluconobacter oxydans or related strains of bacteria expressing mutated RCS 21 nucleic acid and protein molecules such that the yield, productivity, and/or efficiency of production of a desired compound such as Vitamin C is improved.

In connection with the above process using a microorganism, in one aspect, the process of the present invention leads to yields of Vitamin C which are in general at least about more than 5.7 g/l, such as 10 g/l, 20 g/l, 50 g/l, 100 g/l, 200 g/l, 300 g/l, 400 g/l or more than 600 g/l. In one embodiment, the yield of Vitamin C produced by the process of the present invention is in the range of from about more than 5.7 to about 600 g/l. The yield of Vitamin C refers to the concentration of Vitamin C in the harvest stream coming directly out of the production vessel, i.e. the cell-free supernatant comprising the Vitamin C.

In one aspect of the invention, microorganisms (in particular from the genera of Gluconobacter, Gluconacetobacter and Acetobacter) are provided that are able to directly produce Vitamin C from a suitable carbon source like D-sorbitol and/or L-sorbose. When measured for instance in a resting cell method after an incubation period of 20 hours, these organisms were found to be able to produce Vitamin C directly from D-sorbitol or L-sorbose, even up to a level of 280 mg/l and 670 mg/l respectively. In another aspect of the invention, a microorganism is provided capable of directly producing Vitamin C in quantities of 300 mg/l when starting from D-sorbitol or more or 800 mg/l or more when starting from L-sorbose, respectively when for instance measured in a resting cell method after an incubation period of 20 hours. Such may be achieved by increasing the activity of a RCS polypeptide, preferably a RCS 21 polypeptide. The yield of Vitamin C produced from D-sorbitol may even be as high as 400, 600, 1000 mg/l or even exceed 1.5, 2, 4, 10, 20, 50 g/l. The yield of Vitamin C produced from L-sorbose may even be as high as 1000 mg/l or even exceed 1.5, 2, 4, 10, 20, 50 g/l. Preferably, these amounts of Vitamin C can be achieved when measured by resting cell method after an incubation period of 20 hours.

As used herein, measurement in a “resting cell method” comprises (i) growing the cells by means of any method well know to the person skilled in the art, (ii) harvesting the cells from the growth broth, and (iii) incubating the harvested cells in a medium containing the substrate which is to be converted into the desired product, e.g. Vitamin C, under conditions where the cells do not grow any longer, i.e. there is no increase in the amount of biomass during this so-called conversion step.

The recombinant microorganism carrying e.g. a modified RCS 21 gene and which is able to produce the fermentation product in significantly higher yield, productivity, and/or efficiency may be cultured in an aqueous medium supplemented with appropriate nutrients under aerobic conditions as described above.

The nucleic acid molecules, polypeptides, vectors, primers, and recombinant microorganisms described herein may be used in one or more of the following methods: identification of Gluconobacter oxydans and related organisms; mapping of genomes of organisms related to Gluconobacter oxydans; identification and localization of Gluconobacter oxydans sequences of interest; evolutionary studies; determination of RCS 21 protein regions required for function; modulation of a RCS 21 protein activity or function; modulation of the activity of a RCS pathway; and modulation of cellular production of a desired compound, such as Vitamin C.

The invention provides methods for screening molecules which modulate the activity of a RCS 21 protein, either by interacting with the protein itself or a substrate or binding partner of the RCS 21 protein, or by modulating the transcription or translation of a RCS 21 nucleic acid molecule of the invention. In such methods, a microorganism expressing one or more RCS 21 proteins of the invention is contacted with one or more test compounds, and the effect of each test compound on the activity or level of expression of the RCS 21 protein is assessed.

The biological, enzymatic or other activity of RCS proteins can be measured by methods well known to a skilled person, such as, for example, by incubating a membrane fraction or cell-free extract containing the RCS protein in the presence of coenzyme Q2 (CoQ2), an artificial electron acceptor, and by measuring the consumption of oxygen by methods such as the Clark-type oxygen electrode (Rank Brothers, Cambridge, United Kingdom). Thus, for example, the activity of ubiquinol oxidase bd, a cyanide-resistant terminal oxidase, can be measured in an assay where membrane fractions or cell-free extracts containing this enzyme are incubated in the presence of 50 mM phosphate buffer at pH 6.5, 0.02% of the detergent Tween20 and 100 μM cyanide in order to inactivate other cyanide-sensitive oxidases. The enzyme reaction can then be started by addition of 30 mM of the reduced artificial electron acceptor, CoQ_{2red}, and followed by measuring the increase in absorbance at 275 nm. The rate of consumption of oxygen can be measured with help of the Clark-type electrode, and is directly proportional to the ubiquinol oxidase bd activity present in the membrane fraction or in the cell-free extract.

It may be evident from the above description that the fermentation product of the methods according to the invention may not be limited to Vitamin C alone. The “desired compound” or “fermentation product” as used herein may be any natural product of Gluconobacter oxydans, which includes the final products and intermediates of biosynthesis pathways, such as for example L-sorbose, L-sorbosone, D-gluconate, 2-keto-D-gluconate, 5-keto-D-gluconate, 2,5-diketo-D-gluconate and 2-keto-L-gulonate (2-KGA), in particular the biosynthetic generation of Vitamin C.

Thus, the present invention is directed to the use of a polynucleotide, polypeptide, vector, primer and recombinant microorganism as described herein in the production of Vitamin C, i.e., the direct conversion of a carbon source into Vitamin C. In a preferred embodiment, a modified polynucleotide, polypeptide, vector and recombinant microorganism as described herein is used for improving the yield, productivity, and/or efficiency of the production of Vitamin C.

The terms “production” or “productivity” are art-recognized and include the concentration of the fermentation product (for example, Vitamin C) formed within a given time and a given fermentation volume (e.g., kg product per hour per liter). The term “efficiency of production” includes the time required for a particular level of production to be achieved (for example, how long it takes for the cell to attain a particular rate of output of a fermentation product). The term “yield” is art-recognized and includes the efficiency of the conversion of the carbon source into the product (i.e., Vitamin C). This is generally written as, for example, kg product per kg carbon source. By “increasing the yield and/or production/productivity” of the compound it is meant that the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound. The language “transport” or “import” is art-recognized and includes the facilitated movement of one or more molecules across a cellular membrane through which the molecule would otherwise either be unable to pass or be passed inefficiently.

Vitamin C as used herein may be any chemical form of L-ascorbic acid found in aqueous solutions, such as for instance undissociated, in its free acid form or dissociated as an anion. The solubilized salt form of L-ascorbic acid may be characterized as the anion in the presence of any kind of cations usually found in fermentation supernatants, such as for instance potassium, sodium, ammonium, or calcium. Also included may be isolated crystals of the free acid form of L-ascorbic acid. On the other hand, isolated crystals of a salt form of L-ascorbic acid are called by their corresponding salt name, i.e. sodium ascorbate, potassium ascorbate, calcium ascorbate and the like.

In one preferred embodiment, the present invention is related to a process for the production of Vitamin C wherein a nucleotide according to the invention or a modified polynucleotide sequence as described above is introduced into a suitable microorganism, the recombinant microorganism is cultured under conditions that allow the production of Vitamin C in high productivity, yield, and/or efficiency, the produced fermentation product is isolated from the culture medium and optionally further purified.

This invention is further illustrated by the following examples which should not be construed as limiting. The contents of all references, patent applications, patents and published patent applications, cited throughout this application are hereby incorporated by reference.

### EXAMPLES

### Example 1

### Preparation of Chromosomal DNA and Amplification of DNA Fragment by PCR

Chromosomal DNA of Gluconobacter oxydans DSM 17078 was prepared from the cells cultivated at 30° C. for 1 day in mannitol broth (MB) liquid medium consisting of 25 g/l mannitol, 5 g/l of yeast extract (Difco), and 3 g/l of Bactopeptone (Difco) by the method described by Sambrook et al (1989) “Molecular Cloning: A Laboratory Manual/Second Edition”, Cold Spring Harbor Laboratory Press).

A DNA fragment was prepared by PCR with the chromosomal DNA prepared above and a set of primers, Pf (SEQ ID NO:3) and Pr (SEQ ID NO:4). For the reaction, the Expand High Fidelity PCR kit (Roche Diagnostics) and 10 ng of the chromosomal DNA was used in total volume of 100 μl according to the supplier's instruction to have the PCR product containing RCS 21 DNA sequence (SEQ ID NO:1). The PCR product was recovered from the reaction and its correct sequence confirmed.

### Example 2

### Overexpression of the RCS 21 Gene in G. oxydans DSM 17078

To upregulate the expression of the RCS 21 gene, an overexpression system using an integrative construct may be used. Herein, the RCS 21 gene is fused to a strong constitutive promoter, and the construct is then introduced into G. oxydans DSM 17078.

The overexpression of the RCS 21 gene may be determined through standard methods known to those skilled in the art, such as transcript analysis using Northern Blot, RT-PCR or other technology, protein expression determination using Western Blot, two-dimensional gel electrophoresis, protein activity determination using specific enzyme assays or through direct measurement of product formation or substrate conversion.

The promoter can be any promoter that exhibits strong constitutive activity in Gluconobacter oxydans such as the tufB promoter from Escherichia coli, the tufB promoter from Gluconobacter oxydans, the dnaA promoter from Gluconobacter oxydans, or the sndh promoter from Gluconobacter oxydans.

For the overexpression of the RCS 21 gene, the promoter of the RCS 21 gene may be replaced by the strong constitutive modified P_{sndh} promoter (SEQ ID NO:5). In order to achieve this, a DNA fragment is built up by Long Flanking Homology (LFH)-PCR consisting of 500-bp of the upstream region of the RCS 21 gene, a kanamycin-resistance cassette, the P_{sndh}-promoter fused to a modified ribosome binding site and the first 500-bp of the RCS 21 gene. In order to construct the DNA fragment, firstly the single parts are amplified by PCR using the GC-rich PCR kit (Roche Molecular Biochemicals). The RCS 21 DNA upstream region is amplified using primer pair RCS 21US+1 (SEQ ID NO:6) and KmRCS 21US-1 (SEQ ID NO:7) containing complementary kanamycin-resistance cassette overhang sequence at 5′-end. The P_{sndh} promoter fragment is amplified using primer pair KmPsndh+1 (SEQ ID NO:8) containing complementary kanamycin-resistance cassette overhang sequence at 5′-end and RCS 21Psndh-1 (SEQ ID NO:9) containing complementary RCS 21 DNA overhang sequence at 5′-end. The first 500-bp of the RCS 21 gene is amplified using primer pair PsndhRCS 21+1 (SEQ ID NO:10) containing complementary P_{sndh} promoter overhang sequence at 5′-end and RCS 21-1 (SEQ ID NO:11). In these cases G. oxydans DSM 17078 genomic DNA is used as a template. The kanamycin-resistance cassette is amplified using plasmid pUC4K (Amersham Bioscience, accession No. X06404) as a template and primer pair Km+1 (SEQ ID NO:12) and Km−1 (SEQ ID NO:13). The PCR conditions consist of 35 cycles of denaturation at 94° C. for 30 sec, annealing at 50° C. for 30 sec and extension at 72° C. for 1 min. The individual PCR fragments are gel-purified, mixed and re-amplified using the primer pair RCS 21US+1/RCS 21-1 to amplify a full length product whereby the P_{sndh} promoter is inserted upstream of the RCS 21 gene. The PCR reaction conditions for the second round reaction consist of 94° C., 2 min, then 10 cycles of [94° C., 30 sec, 63° C., 30 sec, 68° C., 6 min], followed by 20 cycles of [94° C., 30 sec, 63° C., 30 sec, 68° C., 6 min with an additional 20 sec per cycle] and a final extension at 68° C. for 10 min.

The PCR product is transformed directly into competent G. oxydans DSM 17078 cells and transformants are selected on mannitol broth agar medium containing kanamycin to a final concentration of 50 μg ml⁻¹. Several putative transformants are observed of which several are then analyzed by PCR using primer pair RCS 21US+1/RCS 21-1 to verify that the DNA fragment has inserted into the genome via a double crossover. Strains showing the correct size PCR product have the PCR product sequenced. Strains with the correct sequence are named G. oxydans DSM 17078-RCS 21up1 and G. oxydans DSM 17078-RCS 21up2.

### Example 3

### Production of Vitamin C from D-Sorbitol Using Resting Cells

Cells of G. oxydans DSM 17078, G. oxydans DSM 17078-RCS 21up1 and G. oxydans DSM 17078-RCS 21up2 are grown at 27° C. for 3 days on No. 3BD agar medium containing 70 g/l D-sorbitol, 0.5 g/l glycerol, 7.5 g/l yeast extract (Difco), 2.5 g/l MgSO₄.7H₂O, 10 g/l CaCO₃ and 18 g/l agar (Difco).

Cells are scraped from the agar plates, suspended in distilled water and used for resting cell reactions conducted at 30° C. with shaking at 220 rpm. A series of reactions (0.5 ml reaction mixture in 5 ml reaction tubes) are carried out with 2% D-sorbitol in reaction mixtures further containing 0.3% NaCl, and 1% CaCO₃ and is incubated with cells at a final concentration of OD₆₀₀=10. After an incubation period of 20 hours, samples of the reaction mixtures are analyzed by high performance liquid chromatography (HPLC) using an Agilent 1100 HPLC system (Agilent Technologies, Wilmington, USA) with a LiChrospher-100-RP18 (125×4.6 mm) column (Merck, Darmstadt, Germany) attached to an Aminex-HPX-78H (300×7.8 mm) column (Biorad, Reinach, Switzerland). The mobile phase is 0.004 M sulfuric acid, and the flow rate was 0.6 ml/min. Two signals are recorded using an UV detector (wavelength 254 nm) in combination with a refractive index detector. In addition, the identification of the L-ascorbic acid is done using an amino-column (YMC-Pack Polyamine-II, YMC, Inc., Kyoto, Japan) with UV detection at 254 nm. The mobile phase is 50 mM NH₄H₂PO₄ and acetonitrile (40:60).

An Agilent Series 1100 HPLC-mass spectrometry (MS) system is used to identify L-ascorbic acid. The MS is operated in positive ion mode using the electrospray interface. The separation is carried out using a LUNA-C8(2) column (100×4.6 mm) (Phenomenex, Torrance, USA). The mobile phase is a mixture of 0.1% formic acid and methanol (96:4). L-Ascorbic acid elutes with a retention time of 3.1 minutes. The identity of the L-ascorbic acid is confirmed by retention time and the molecular mass of the compound.

The supernatants of the reaction mixtures incubated with cells of G. oxydans DSM 17078-RCS 21up1 and G. oxydans DSM 17078-RCS 21up2 contain at least 20% more Vitamin C than the supernatant of the reaction mixture incubated with cells of G. oxydans DSM 17078.

### Example 4

### Presence of the RCS 21 Gene and Equivalents in Other Organisms

The presence of SEQ ID NO:1 and/or equivalents in other organisms than the ones disclosed herein before, e.g. organisms as mentioned in Table 1, may be determined by a simple DNA hybridization experiment.

Strains of Acetobacter aceti subsp. xylinum IFO 13693 and IFO 13773 are grown at 27° C. for 3 days on No. 350 medium containing 5 g/l Bactopeptone (Difco), 5 g/l yeast extract (Difco), 5 g/l glucose, 5 g/l mannitol, 1 g/l MgSO₄.7H₂O, 5 ml/l ethanol, and 15 g/l agar. All other Acetobacter, Gluconacetobacter and all Gluconobacter strains are grown at 27° C. for 3 days on mannitol broth (MB) agar medium containing 25 g/l mannitol, 5 g/l yeast extract (Difco), 3 g/l Bactopeptone (Difco), and 18 g/l agar (Difco). E. coli K-12 is grown on Luria Broth agar medium. The other strains are grown on medium recommended by the suppliers or according to methods known in the art. Genomic DNA is extracted as described by e.g. Sambrook et al., 1989, “Molecular Cloning: A Laboratory Manual/Second Edition”, Cold Spring Harbor Laboratory Press) from a suitable organism as, e.g. mentioned in Table 1.

Genomic DNA preparations are digested with restriction enzymes such as EcoRI or HindIII, and 1 μg of the DNA fragments are separated by agarose gel electrophoresis (1% agarose). The gel is treated with 0.25 N HCl for 15 min and then 0.5 N NaOH for 30 min, and then blotted onto nitrocellulose or a nylon membrane with Vacuum Blotter Model 785 (BIO-RAD Laboratories AG, Switzerland) according to the instruction of the supplier. The resulting blot is then brought into contact/hybridized with a solution wherein the probe, such as e.g. a DNA fragment with SEQ ID NO:1 sequence or a DNA fragment containing the part or whole of the SEQ ID NO:1 sequence to detect positive DNA fragment(s) from a test organism. A DIG-labeled probe, e.g. SEQ ID NO:1, may be prepared according to Example 1 by using the PCR-DIG labeling kit (Roche Diagnostics) and a set of primers, SEQ ID NO:3 and SEQ ID NO:4. A result of such a blot is depicted in Table 1.

The hybridization may be performed under stringent or highly stringent conditions. A preferred, non-limiting example of such conditions are hybridization in 6× sodium chloride/sodium citrate (SSC) at about 45° C., followed by one or more washes in 1×SSC, 0.1% SDS at 50° C., preferably at 55° C., more preferably at 60° C. and even more preferably at 65° C. Highly stringent conditions include, for example, 2 h to 4 days incubation at 42° C. in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 min in 2×SSC and 0.1% SDS at room temperature and then washing twice for 15-30 min in 0.5×SSC and 0.1% SDS or 0.1×SSC and 0.1% SDS at 65-68° C. To detect DNA fragments with lower identity to the probe DNA, final washing steps can be done at lower temperatures such as 50-65° C. and for shorter washing time such as 1-15 min.

The genes corresponding to the positive signals within the respective organisms shown in Table 1 can be cloned by a PCR method well known in the art using genomic DNA of such an organism together with a suitable primer set, such as e.g. SEQ ID NO:3 and SEQ ID NO:4 under conditions as described in Example 1 or as follows: 5 to 100 ng of genomic DNA is used per reaction (total volume 50 μl). Expand High Fidelity PCR system (Roche Diagnostics) can be used with reaction conditions consisting of 94° C. for 2 min; 30 cycles of (i) denaturation step at 94° C. for 15 sec, (ii) annealing step at 60° C. for 30 sec, (iii) synthesis step at 72° C. for 0.5 to 5 min depending to the target DNA length (1 min/1 kb); extension at 72° C. for 7 min. Alternatively, one can perform a PCR with degenerate primers, which can be synthesized based on SEQ ID NO:2 or amino acid sequences as consensus sequences selected by aligning several amino acid sequences obtained by a sequence search program such as BLASTP (or BLASTX when nucleotide sequence is used as a “query sequence”) to find proteins having a similarity to the protein of SEQ ID NO:2. For PCR using degenerate primers, temperature of the second annealing step (see above) can be lowered to 55° C., or even to 50-45° C. A result of such an experiment is shown in Table 1.

Samples of the PCR reactions are separated by agarose gel electrophoresis and the bands are visualized with a transilluminator after staining with e.g. ethidium bromide, isolated from the gel and the correct sequence is confirmed.

Consensus sequences mentioned above might be amino acid sequences belonging to certain categories of several protein domain/family databases such as PROSITE (database of protein families and domains), COGs (Cluster of Ortholog Groups), CDD (Conserved Domain Databases), pfam (large collection of multiple sequence alignments and hidden Markov models covering many common protein domains and families). Once one can select certain protein with identical/similar function to the protein of this invention from proteins containing domain or family of such databases, corresponding DNA encoding the protein can be amplified by PCR using the protein sequence or its nucleotide sequence when it is available in public databases. Following organisms may further provide genes, which can be used as an alternative gene of this invention: Xanthomonas campestris pv. campestris ATCC 33913, Xanthomonas oryzae pv. oryzae KACC 10331, Agrobacterium tumefaciens C58, Bradyrhizobium japonicum USDA 110 or Sinorhizobium meliloti 1021.

### Example 5

### Overexpression of the RCS 21 Gene and Equivalents from Other Organisms for Production of Vitamin C

In order to improve Vitamin C production in a suitable microorganism which is capable to directly produce Vitamin C from a given substrate, the RCS 21 gene and equivalents as, e.g. a PCR product obtained in Example 4, referred to herein as gene X, can be used in an overexpression system according to Example 2 or can be cloned into pCR2.1-TOPO (Invitrogen, Carlsbad, Calif., USA) and used to transform E. coli TG1 to have a Apr transformant carrying pCR2.1-TOPO-gene X, i.e. carrying a PCR product obtained in Example 4. The insert is amplified with a set of primers, PfNdeI [SEQ ID NO:3 with CCCAT at the 5′-end] and PrHindIII [SEQ ID NO:4 with CCAAGCTT at the 5′-end], by PCR. Resulting PCR product is digested with NdeI and HindIII and the fragment is inserted together with PcrtE-SD (Shine-Dalgarno) fragment (WO 02/099095) digested with XhoI and NdeI into pVK100 (ATCC 37156) between the sites of XhoI and HindIII. E. coli TG1 is transformed with the ligation product to have Tc^{r} transformant carrying plasmid pVK-PcrtE-SD-gene X, which is then used to transform a suitable host, e.g. G. oxydans DSM 17078 by electroporation to have e.g. Tc^{r} G. oxydans DSM 17078/pVK-PcrtE-SD-gene X.

Production of Vitamin C using the recombinant cells of e.g. G. oxydans strains DSM 17078 and the corresponding wild-type strain are performed according to Example 3.

In the resting cell reaction with 1% L-sorbosone as the substrate, the recombinant cells can produce at least more than 20% Vitamin C compared to the wild-type strain.

TABLE 1
Equivalents of the RCS 21 gene in other organisms.

| Strain | Signal 1 | Signal 2 | Signal 3 |
|---|---|---|---|
| G. oxydans DSM 17078 | ++++ | + | + |
| G. oxydans IFO 3293 | ++++ | + | + |
| G. oxydans IFO 3292 | ++++ | + | + |
| G. oxydans ATCC 621H | ++++ | + | + |
| G. oxydans IFO 12528 | ++++ | + | + |
| G. oxydans G 624 | ++++ | + | + |
| G. oxydans T-100 | ++++ | + | + |
| G. oxydans IFO 3291 | ++++ | + | + |
| G. oxydans IFO 3255 | ++++ | + | + |
| G. oxydans ATCC 9937 | ++++ | + | + |
| G. oxydans IFO 3244 | ++++ | + | + |
| G. cerinus IFO 3266 | +++ | + | + |
| G. frateurii IFO 3260 | +++ | + | + |
| G. oxydans IFO 3287 | ++++ | + | + |
| Acetobacter aceti subsp. orleanus IFO 3259 | ++ | − | + |
| Acetobacter aceti subsp. xylinum IFO 13693 | ++ | − | + |
| Acetobacter aceti subsp. xylinum IFO 13773 | ++ | − | + |
| Acetobacter sp. ATCC 15164 | ++ | − | + |
| G. thailandicus NBRC 100600 | +++ | + | + |
| Gluconacetobacter liquefaciens | ++ | + | + |
| ATCC 14835 | | | |
| Gluconacetobacter polyoxogenes NBI 1028 | ++ | + | + |
| Gluconacetobacter diazotrophicus | ++ | + | + |
| ATCC 49037 | | | |
| Gluconacetobacter europaeus DSM 6160 | ++ | + | + |
| Acetobacter aceti 1023 | ++ | − | + |
| Acetobacter pasteurianus NCI 1193 | ++ | − | + |
| Pseudomonas putida ATCC 21812 | + | − | + |
| Pseudomonas aeruginosa PAO1 | + | − | + |
| Pseudomonas fluorescens DSM 50106 | + | − | + |
| Pseudomonas syringae B728a | + | − | + |
| Paracoccus denitrificans strain Pd1222 | + | − | + |
| Rhodopseudomonas palustris CGA009 | + | − | + |
| Pantoea citrea 1056R | − | − | − |
| E. coli K-12 | − | − | − |
| Saccharomyces cerevisiae | − | − | − |
| Aspergillus niger | − | − | − |
| Mouse | − | − | − |

Signal 1: Detection of DNA on a blot with genomic DNA of different strains and SEQ ID NO: 1 as labeled probe.
Signal 2: Detection of DNA of different strains in a PCR reaction using primer pair SEQ ID NO: 3 and SEQ ID NO: 4.
Signal 3: Detection of DNA of different strains in a PCR reaction using degenerate primers. For more explanation refer to the text.

1. A polynucleotide selected from the group consisting of:
(a) polynucleotides encoding a polypeptide comprising the amino acid sequence according to SEQ ID NO: 2;
(b) polynucleotides comprising the nucleotide sequence according to SEQ ID NO: 1;
(c) polynucleotides comprising a nucleotide sequence obtainable by nucleic acid amplification such as polymerase chain reaction, using genomic DNA from a microorganism as a template and a primer set according to SEQ ID NO: 3 and SEQ ID NO: 4;
(d) polynucleotides comprising a nucleotide sequence encoding a fragment or derivative of a polypeptide encoded by a polynucleotide of any of (a) to (c) wherein in said derivative one or more amino acid residues are conservatively substituted compared to said polypeptide, and said fragment or derivative has the activity of a PQQ biosynthesis protein;
(e) polynucleotides the complementary strand of which hybridizes under stringent conditions to a polynucleotide as defined in any one of (a) to (d) and which encode a PQQ biosynthesis protein; and
(f) polynucleotides which are at least 70%, such as 85, 90 or 95% identical to a polynucleotide as defined in any one of (a) to (d) and which encode a PQQ biosynthesis protein
or
the complementary strand of such a polynucleotide.

2. A vector containing the polynucleotide according to claim 1.

3. The vector of claim 2 in which the polynucleotide is operatively linked to expression control sequences allowing the expression in prokaryotic or eukaryotic host cells.

4. A microorganism genetically engineered with a polynucleotide according to claim 1 or with a vector containing the polynucleotide.

5. A microorganism according to claim 4 capable of directly producing Vitamin C from D-sorbitol in quantities of 300 mg/l or more when measured in a resting cell method after an incubation period of 20 hours.

6. A microorganism according to claim 5 capable of directly producing Vitamin C from L-sorbose in quantities of 800 mg/l or more.

7. A polypeptide encoded by a polynucleotide according to claim 1.

8. Process for producing cells capable of expressing a polypeptide encoded by a polynucleotide according to claim 1, comprising the step of genetically engineering cells with a vector containing the polynucleotide or with a polynucleotide.

9. Use of a polynucleotide according to claim 1 or a vector containing the polynucleotide for the production of Vitamin C and/or 2-KGA.

10. Use according to claim 9, wherein the polynucleotide is operatively linked to expression control sequences and transferred into a microorganism.

11. Use according to claim 10, wherein the expression control sequences comprise a regulation-, and/or promoter-, and/or terminator sequence and wherein at least one of these sequences is altered in such a way that it leads to an improved yield and/or efficiency of production of Vitamin C and/or 2-KGA produced by said microorganism.

12. Use according to claim 11, wherein the expression control sequences comprise a regulation-, and/or promoter-, and/or terminator sequence and wherein at least one of these sequences is altered in such a way that it leads to an increased and/or improved activity of a PQQ biosynthesis protein.

13. A microorganism genetically engineered with a polynucleotide according to claim 1, or with a vector containing the polynucleotide or a microorganism containing an endogenous gene comprising the polynucleotide, said microorganism being genetically altered in such a way that it leads to an improved yield and/or efficiency of production of Vitamin C and/or 2-KGA produced by said microorganism.

14. A microorganism genetically engineered with a polynucleotide according to claim 1, or with a vector containing the polynucleotide or a microorganism containing an endogenous gene comprising the polynucleotide, said microorganism being genetically altered in such a way that it leads to an improved yield and/or efficiency of production of Vitamin C and/or 2-KGA produced by said microorganism and producing a polypeptide encoded by the polynucleotide with increased and/or improved PQQ biosynthesis protein activity.

15. A microorganism genetically engineered with a polynucleotide according to claim 1 or with a vector containing the polynucleotide, wherein the polynucleotide is overexpressed.

16. A microorganism genetically engineered with a polynucleotide according to claim 1 or with a vector containing the polynucleotide according to claim 1 selected from the group consisting of Pseudomonas, Pantoea, Escherichia, Corynebacterium, Ketogulonicigenium and acetic acid bacteria like e.g., Gluconobacter, Acetobacter or Gluconacetobacter, preferably Acetobacter sp., Acetobacter aceti, Gluconobacter frateurii, Gluconobacter cerinus, Gluconobacter thailandicus, Gluconobacter oxydans, preferably Gluconobacter oxydans, more preferably Gluconobacter oxydans DSM 17078.

17. Process for the production of an enhanced endogenous gene encoding a PQQ biosynthesis protein in a microorganism, said microorganism comprising a polynucleotide according to claim 1, said process comprising the step of altering said polynucleotide in such a way that it leads to an improved yield and/or efficiency of production of Vitamin C and/or 2-KGA produced by said microorganism.

18. Process for the production of a microorganism capable of producing Vitamin C and/or 2-KGA, comprising the step of altering said microorganism so that the microorganism produces a polypeptide with increased and/or improved PQQ biosynthesis protein activity leading to an improved yield and/or efficiency of production of Vitamin C and/or 2-KGA produced by said microorganism.

19. Process for the production of a microorganism containing an endogenous gene comprising a polynucleotide according to claim 1, comprising the step of altering said microorganism so that the endogenous gene is overexpressed, leading to an improved yield and/or efficiency of production of Vitamin C and/or 2-KGA produced by said microorganism.

20. Process for the production of a microorganism capable of producing Vitamin C and/or 2-KGA, comprising the step of altering said microorganism so that the microorganism produces a polypeptide with increased and/or improved PQQ biosynthesis protein activity leading to an improved yield and/or efficiency of production of Vitamin C and/or 2-KGA produced by said microorganism for the production of a microorganism according to claim 13.

21. Process for the production of Vitamin C and/or 2-KGA with a microorganism according to claim 13 wherein said microorganism is cultivated in a aqueous nutrient medium under conditions that allow the direct production of Vitamin C and/or 2-KGA from D-sorbitol or L-sorbose and wherein optionally Vitamin C and/or 2-KGA is isolated as the fermentation product.
